(19) United States
(12) Patent Application Publication
(10) Pub. No.: US 2016/0303133 A1
(43) Pub. Date: Oct. 20, 2016

(54) TREATMENT OF GASTROINTESTINAL AND OTHER DISORDERS

(71) Applicant: NUMEDII, INC., Palo Alto, CA (US)

(72) Inventors: Joel DUDLEY, Rye, NY (US); Carol CHRISTOPHER, Palo Alto, CA (US); Pankaj PASRICHA, Columbia, MD (US)

(73) Assignee: NUMEDII, INC., Paloalto, CA (US)

(21) Appl. No.: 14/782,304

(22) PCT Filed: Apr. 4, 2014
(86) PCT No.: PCT/US14/33080

§ 371 Date: Jun. 2, 2016

Related U.S. Application Data
(60) Provisional application No. 61/809,148, filed on Apr. 5, 2013.

(51) Int. Cl. A61K 31/5355; A61K 31/165; A61K 9/00; A61K 31/166; A61K 31/145; A61K 31/138; A61K 31/167

(52) U.S. Cl. CPC A61K 31/5355; A61K 31/138; A61K 31/165; A61K 31/167; A61K 31/166; A61K 31/145; A61K 9/0014; A61K 9/0053; A61K 9/0031

(57) ABSTRACT

Provided herein are methods and compositions for treating gastrointestinal disorders using beta blockers. Compositions comprising Beta blockers such as Timolol and Nadolol are used to treat diseases including ulcerative colitis, IBD and Crohn's disease. The Compositions are administered orally or rectally, at doses that provide a peak plasma concentration of less than 10 ng/ml.

### CROSS-REFERENCE

This application claims the benefit of U.S. Provisional Application No. 61/809,148, filed Apr. 5, 2013, which application is incorporated herein by reference in its entirety and for all purposes.

### FIELD OF THE INVENTION

The present invention is directed to the use of a beta blocker to treat a gastrointestinal disease in a subject in need thereof, comprising administering to the subject a beta blocker. For example, the gastrointestinal disease is inflammatory bowel disorder (IBD) or ulcerative colitis (UC).

### BACKGROUND OF THE INVENTION

Gastrointestinal diseases include a broad range of disorders related to the digestive tract. For example, common gastrointestinal diseases include inflammation and ulcers of any part of the digestive tract such as the esophagus, stomach, or duodenum. A significant fraction of the world population experiences one or more gastrointestinal diseases at some time during their lives. For example, it is estimated that as many as 1.4 million people in the United States suffer from Crohn's disease. Such disorders impose a significant disease burden on the United States healthcare system, with an estimated cost of more than $1.7 billion annually for inflammatory bowel disorders alone. Therefore, there remains a significant need for the treatment and management of gastrointestinal diseases and other disorders mediated by related biological pathways.

Ulcerative colitis is a disease of the colon wherein it is ulcerated and a symptomatic patient may have diarrhea. UC is treated as an autoimmune disease with anti-inflammatory or immunosuppressive agents, including biological therapeutics that targeting specific components of the immune response. Patients may not respond such treatment.

Beta blockers including nadolol, timolol, harmalol, levobunolol, bisoprolol, carteolol, pindolol, metoprolol, acebutolol, propranolol, atenolol, sotalol, penbutolol, labetalol, pronetalol, oxprenolol, practolol, betaxolol, and dexpropranolol are known for being able to decrease heart rate and blood pressure. There is no teaching of beta blockers, including nadolol or timolol, as agents for treating ulcerative colitis.

Steroids such as budesonide, dexamethasone, hydrocortisone, methylprednisolone, prednisolone, prednisone and triamcinolone are known agents for treating ulcerative colitis.

However, long-term usage of corticosteroids is often accompanied by numerous systemic side effects, such as osteoporosis, diabetes mellitus, systemic hypertension, psychiatric disorders and altered steroid metabolism.

### SUMMARY OF THE INVENTION

In one aspect, the invention provides a method of treating a gastrointestinal disease in a subject in need thereof, comprising administering to the subject a beta blocker.

In some embodiments, the beta blocker is selected from the group consisting of nadolol, timolol, harmalol, levobunolol, bisoprolol, alprenolol, carteolol, pindolol, metoprolol, acebutolol, S-propranolol, (−)-atenolol, sotalol, propranolol, R-atenolol, penbutolol, labetalol, pronetalol, oxprenolol, practolol, betaxolol, and dexpropranolol.

In some embodiments, the beta blocker is a compound having the structure:

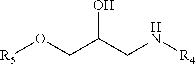

wherein R₄ is H or optionally substituted C₁-C₆ alkyl;
R₅ is optionally substituted aryl, substituted heteroaryl, or optionally substituted cycloalkyl; or
a pharmaceutically acceptable salt or a pharmaceutically acceptable prodrug thereof. For example, R₄ is iso-propyl or tert-butyl. In some embodiments, R₅ is optionally substituted aryl or heteroaryl, for example, R₅ is optionally substituted phenyl, indolyl, naphthyl, thiadiazyl, or R₅ is aryl optionally substituted with one or more substituents chosen from the group consisting of OH, cyano, C₁-C₆ heterocyclo, R₆, —OR₆, —CONH₂, —NHC(O)R₆, —R₆—OR₆, and —R₆—O—R₆—OR₆; wherein each R₆ is independently C₁-C₆ alkyl or C₁-C₆ cycloalkyl.

The invention also provides a method of treating a gastrointestinal disease in a subject in need thereof, comprising administering to the subject a compound of Formula I, a pharmaceutically acceptable salt thereof, or a pharmaceutically acceptable prodrug thereof:

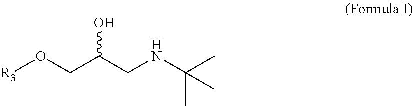

wherein R₃ is

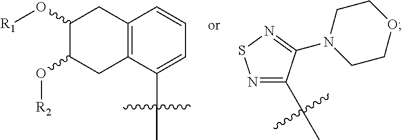

and
R₁ and R₂ are independently H or C₁-C₆ alkyl. For example, in some embodiments, the compound is:

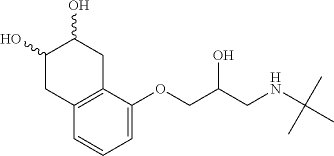 [SMILES: CC(C)(C)NCC(O)COc1cccc2c1CC(O)C(O)C2]

or a pharmaceutically acceptable salt thereof. In other embodiments, the compound is

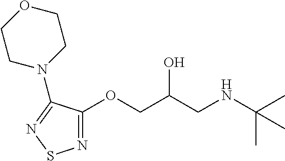 [SMILES: CC(C)(C)NCC(O)COc1nsnc1N1CCOCC1]

or a pharmaceutically acceptable salt thereof.

In some embodiments, the subject is a mammal, such as a human. In some embodiments, the gastrointestinal disease is inflammatory bowel disease such as Crohn's disease or colitis, for instance, ulcerative colitis. In other embodiments, the disease comprises an autoimmune response or an inflammatory response. In some embodiments, the subject is at risk of developing ulcers or gastrointestinal bleeding. In some embodiments, the gastrointestinal disease is prevented from progressing in said subject. In some embodiments, the subject is not in need of treatment for hypertension, angina, migraine headache, irregular heartbeat, or Parkinson's disease.

In some embodiments, the administering step results in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject.

In some embodiments, the administering step occurs prior to, concurrent with, or after administration of an additional treatment regimen or therapeutic agent to said subject. The additional therapeutic agent is, for instance, a steroid or other anti-inflammatory drug. In some embodiments, the additional treatment regimen comprises traditional or laparoscopic surgery. In other embodiments, the additional treatment is a laxative or an anti-constipation agent. In other embodiments, the additional therapeutic agent is antacid or other acidic reducer.

In another aspect, within the methods provided herein, the compound is administered via pH-dependent release, via microbially-triggered delivery, as a conjugate, via time-controlled delivery, via osmotically-regulated delivery, administered via pressure-controlled delivery, via multi matrix systems delivery, via bioadhesion delivery, or via multiparticulate delivery.

In another aspect, within the methods provided herein, the compound is released preferentially in the small or large intestine, colon or rectum, in the stomach, esophagus, for example. In some embodiments, at least 60%, 70%, 80%, or 90% of the administered dose of the compound is released.

The invention further provides a pharmaceutically acceptable composition comprising a compound of Formula I or a pharmaceutically acceptable salt thereof:

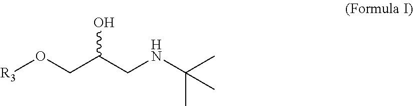

wherein R₃ is

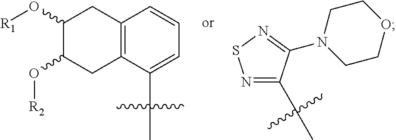

and
R₁ and R₂ are independently H or C₁-C₆ alkyl,
wherein the composition is formulated for oral or rectal administration. In some embodiments, the compound is:

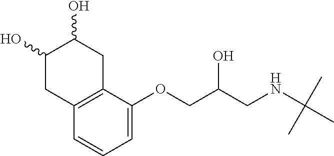 [SMILES: CC(C)(C)NCC(O)COc1cccc2c1CC(O)C(O)C2]

or a pharmaceutically acceptable salt thereof. In other embodiments, the compound is:

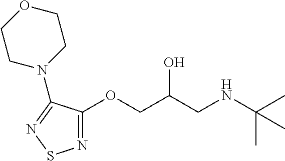 [SMILES: CC(C)(C)NCC(O)COc1nsnc1N1CCOCC1]

or a pharmaceutically acceptable salt thereof.

In some embodiments of the composition provided herein, the compound is formulated for administration via pH-dependent release delivery, microbially-triggered delivery, time-controlled delivery, osmotically-regulated delivery, pressure-controlled delivery, multi matrix systems delivery, bioadhesion delivery, or multiparticulate delivery. In some embodiments, the compound is formulated for release in the small or large intestine, colon or rectum, in the stomach, esophagus, for example.

Also provided is a method of treating a gastrointestinal disease in a subject in need thereof, comprising administering to the subject a pharmaceutical composition comprising a beta blocker; wherein the pharmaceutical composition is delivered by rectal administration. The pharmaceutical composition of the method is, for example, chosen from the group consisting of enemas, rectal gels, rectal foams, rectal aerosols, suppositories, jelly suppositories, or retention enemas. In some embodiments, the pharmaceutical composition is a suppository or a rectal gel. In some embodiments, the pharmaceutical composition comprises less than 40, 20, 10, 8, 6, 4, or 2 mg of beta blocker. In some embodiments, the subject is administered less than 40, 20, 10, 8, 6, 4, or 2 mg of beta blocker daily. In some embodiments, the subject is administered less than 0.6, 0.4, 0.2, 0.1, 0.05, 0.02, or 0.01 mg of beta blocker per kg of the subject's body weight daily.

In some embodiments of the method provided herein, the subject is a mammal, for instance a human. In some embodiments, the gastrointestinal disease is inflammatory bowel disease, Crohn's disease, or colitis, for instance, ulcerative colitis. In other embodiments, the disease comprises an autoimmune response or an inflammatory response. In some embodiments, the subject is at risk of developing ulcers or gastrointestinal bleeding. In some embodiments, the gastrointestinal disease is prevented from progressing in said subject. In some embodiments, the administering step results in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject.

In some embodiments of the method provided herein, the pharmaceutical composition comprises an amount of beta blocker, which, when administered to the subject orally, is insufficient to result in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject. In other embodiments, the pharmaceutical composition comprises an amount of beta blocker which is sufficient to result in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject. In some embodiments, the improvement results in a decrease of colonic score by at least 1, 2, 3, 4, or 5. In some embodiments, the improvement results in a decrease of colonic score by at least 3 or 4. In some embodiments, the improvement is a decrease in colonic score by 1 or 2. In some embodiments of the method, administration of the pharmaceutical composition results in a peak plasma level in the subject of 50, 40, 30, 20, or 10 ng/mL, for example, less than 50, 30 or 10 ng/mL.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a shows the effect of oral (“PO”) treatment with Nadolol on colon weight and length in a prophylactic TNBS rat colitis model. FIG. 1b shows the effect of intra-rectal (“IR”) treatment with Nadolol on colon weight and length in a prophylactic TNBS rat colitis model.

FIG. 2a shows the effect of oral treatment with Nadolol on colonic parameters in a prophylactic TNBS rat colitis model. FIG. 2b shows the effect of intra-rectal treatment with Nadolol on colonic parameters in a prophylactic TNBS rat colitis model.

FIG. 3a shows the effect of oral treatment with Nadolol on colonic score in a prophylactic TNBS rat colitis model. FIG. 3b shows the effect of intra-rectal treatment with Nadolol on colonic score in a prophylactic TNBS rat colitis model.

FIG. 4 shows magnified pictures of colons from prophylactic TNBS rat colitis model. FIG. 4a shows colon from an animal that was not given TNBS and has no lesions. FIG. 4b shows colon from a PO vehicle control animal (with the approximate mean summed score for the group) has severe necrosis (N) and inflammation. FIG. 4c shows colon from a diseased animal given 10 mpk of prednisolone (with the approximate mean summed score for the group) has very minimal necrosis (“N”) and minimal inflammation. FIG. 4d shows colon from a diseased animal given 60 mpk of Nadolol (with the approximate mean summed score for the group) which has moderate necrosis (N) and inflammation.

FIG. 5 shows magnified pictures of colons from prophylactic TNBS rat colitis model. FIG. 5a shows colon from a diseased animal given 120 mpk of Nadolol (with the approximate mean summed score for the group) which has mild necrosis (N) and moderate inflammation. FIG. 5b shows colon from an IR vehicle control animal (with the approximate mean summed score for the group) which has mild necrosis (N) and marked inflammation. FIG. 5c shows colon from a diseased animal given 60 mpk of Nadolol (with the approximate mean summed score for the group) which has moderate necrosis (N) and moderate inflammation. FIG. 5d shows colon from a diseased animal given 120 mpk of Nadolol (with the approximate mean summed score for the group) which has mild necrosis (N) and mild inflammation.

FIG. 6 shows mean histopathology scores in necrosis and inflammation for each treatment group in the prophylactic TNBS rat colitis model.

FIG. 7 shows necrosis ratio (mean plus/minus SE) for each treatment group in the prophylactic TNBS rat colitis model.

FIG. 8 shows summed mean histopathology score (mean plus/minus SE) for each treatment group in the prophylactic TNBS rat colitis model.

FIG. 9a shows the effect of oral treatment with Nadolol at various doses on colon weight and length in a prophylactic TNBS rat colitis model. FIG. 9b shows the effect of intra-rectal treatment with Nadolol at various doses on colon weight and length in a prophylactic TNBS rat colitis model.

FIG. 10a shows the effect of oral treatment with Nadolol at various doses on colonic parameters in a prophylactic TNBS rat colitis model. FIG. 10b shows the effect of intra-rectal treatment with Nadolol at various doses on colonic parameters in a prophylactic TNBS rat colitis model.

FIG. 11a shows the effect of oral treatment with Nadolol at various doses on colonic score in a prophylactic TNBS rat colitis model. FIG. 11b shows the effect of intra-rectal treatment with Nadolol at various doses on colonic score in a prophylactic TNBS rat colitis model.

FIG. 12 shows magnified pictures of colons from prophylactic TNBS rat colitis model. FIG. 12a shows colon from an animal that was not exposed to TNBS and has no lesions. FIG. 12b shows colon section from a PO vehicle control animal (with the approximate mean summed score for the group) which has marked necrosis (N) and inflammation. FIG. 12c shows colon from a diseased animal treated PO with 10 mpk of Prednisolone (with the approximate mean summed score for the group) which has minimal necrosis (N) and inflammation. FIG. 12d shows colon from a diseased animal treated PO with 6 mpk of Nadolol (with the approximate mean summed score for the group) which has severe necrosis (N) and mild inflammation. FIG. 12e shows colon from a diseased animal treated PO with 19 mpk of Nadolol (with the approximate mean summed score for the group) which has marked necrosis (N) and inflammation FIG. 12f shows colon section from a diseased animal treated PO with 60 mpk of Nadolol (with the approximate mean summed score for the group) which has severe necrosis (N) and inflammation. FIG. 12g shows colon section from a diseased animal treated PO with 120 mpk of Nadolol (with the approximate mean summed score for the group) which has no necrosis and mild inflammation. FIG. 12h shows colon section an IR vehicle control animal (with the approximate mean summed score for the group) which has moderate necrosis (N) and marked inflammation. FIG. 12i shows colon section from a diseased animal treated IR with 6 mpk of Nadolol (with the approximate mean summed score for the group) which has no necrosis and mild inflammation. FIG. 12j shows colon section from a diseased animal treated IR with 19 mpk of Nadolol (with the approximate mean summed score for the group) which has mild necrosis (N) and moderate inflammation. FIG. 12k shows colon section from a diseased animal treated IR with 60 mpk of Nadolol (with the approximate mean summed score for the group) which has severe necrosis and inflammation.

FIG. 13 shows mean histopathology scores in necrosis and inflammation for each PO treatment group in a prophylactic TNBS rat colitis model.

FIG. 14 shows mean histopathology scores in necrosis and inflammation for each IR treatment group in a prophylactic TNBS rat colitis model

FIG. 15 shows summed mean histopathology score (mean plus/minus SE) for each PO treatment group in a prophylactic TNBS rat colitis model.

FIG. 16 shows summed mean histopathology score (mean plus/minus SE) for each IR treatment group in a prophylactic TNBS rat colitis model.

FIG. 17 shows the necrosis ratio to total length (mean plus/minus SE) for each PO treatment group in a prophylactic TNBS rat colitis model.

FIG. 18 shows the necrosis ratio to total length (mean plus/minus SE) for each IR treatment group in a prophylactic TNBS rat colitis model.

FIG. 19a shows the exposure of Nadolol (Plasma Nadolol, ng/mL) over time for a diseased rat TNBS rat colitis model dosed with 120 mpk Nadolol (“TNBS 120”) PO and a healthy rat (“V 120”) dosed with 120 mpk Nadolol PO. FIG. 19b shows the exposure of Nadolol (Plasma Nadolol, ng/mL) over time for a diseased rat TNBS rat colitis model dosed with 120 mpk Nadolol (“TNBS 120”) IR and a healthy rat (“V 120”) dosed with 120 mpk Nadolol IR. FIG. 19c shows the exposure of Nadolol (Plasma Nadolol, ng/mL) over time for a diseased rat TNBS rat colitis model dosed with 19 mpk Nadolol (“TNBS 19”) PO and a healthy rat (“V 19”) dosed with 19 mpk Nadolol PO. FIG. 19d shows the exposure of Nadolol (Plasma Nadolol, ng/mL) over time for a diseased rat TNBS rat colitis model dosed with 19 mpk Nadolol (“TNBS 19”) IR and a healthy rat (“V 19”) dosed with 19 mpk Nadolol IR

FIG. 20 shows the average colonic score of rats in a TNBS colitis model treated with various beta blockers oral administration (PO) versus prednisolone or vehicle (+TNBS).

FIG. 21 shows the average colonic score of rats in a TNBS colitis model treated with various beta blockers Intra-rectal administration (IR) versus prednisolone or vehicle (+TNBS).

FIG. 22 shows the histopathology average score for colonic necrosis, colonic inflammation, and sum for rats in a TNBS colitis model treated with various beta blockers Intra-rectal administration (IR) versus prednisolone or vehicle (+TNBS) or healthy control (No TNBS).

FIG. 23 shows the effect of treatment on the histopathology ratio score for rats in a TNBS colitis model treated with various beta blockers Intra-rectal administration (IR) versus prednisolone or vehicle (+TNBS) or healthy control (No TNBS).

### DETAILED DESCRIPTION OF THE INVENTION

### Definitions

As used above, and throughout the description of the invention, the following abbreviations, unless otherwise indicated, shall be understood have the following meanings.

The term “gastrointestinal disease” as used herein refers to all diseases or disorders that pertain to the gastrointestinal tract. This includes diseases of the esophagus, stomach, first, second, and third part of the duodenum, jejunum, ileum, the ileo-cecal complex, large intestine (ascending, transverse, and descending colon), sigmoid colon, rectum, and anus.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “beta blocker” (can also be called a β-blocker, beta-adrenergic blocking agent, beta antagonist, beta-adrenergic antagonists, beta-adrenoreceptor antagonists, or beta adrenergic receptor antagonists) refers to a compound that binds to a beta receptor. Some beta blockers are members of the class of drugs that target at least one beta receptor. Beta blockers can have high affinity for one or more beta receptors. For instance, some beta blockers can bind multiple beta receptors with similar affinity. Other beta blockers selectively bind one beta receptor with higher affinity than other beta receptors. Non-limiting examples of a beta blocker include nadolol, timolol, harmalol, levobunolol, bisoprolol, alprenolol, carteolol, pindolol, metoprolol, acebutolol, S-propranolol, (−)-atenolol, sotalol, propranolol, R-atenolol, penbutolol, labetalol, pronetalol, oxprenolol, practolol, betaxolol, and dexpropranolol.

The term “effective amount”, “effective therapeutic amount” or “therapeutically effective amount” refers to that amount of compound or biological agent that is sufficient to effect the intended applications, including without limitation, clinical results such as reducing bleeding, reducing inflammation, reducing diarrhea, reducing constipation, inhibiting ulcer formation, restoring digestive function, decreasing symptoms resulting from the disease, increasing the quality of life of those suffering from the disease, enhancing the effect of another medication, delaying the progression of the disease, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” refers to the relatively non-toxic, inorganic and organic acid addition salts of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-beta-hydroxynaphthoates, gentisates, isethionates, di-p-toluoyltartrates, methanesulphonates, ethanesulphonates, benzenesulphonates, p-toluenesulphonates, cyclohexylsulphamates and quinateslauryl-sulphonate salts, and the like. See, for example S. M. Berge, et al., “Pharmaceutical Salts,” J. Pharm. Sci., 66, 1-19 (1977) which is incorporated herein by reference.

The term “inhibit,” as used herein, refers to the ability of a compound or any agent to reduce or impede a described function, level, activity, synthesis, release, binding, etc., based on the context in which the term “inhibit” is used. The term “inhibit” is used interchangeably with “reduce,” “block,” and “decrease.”

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication, amelioration or stabilization of the underlying disorder being treated (e.g., in the context of many gastrointestinal diseases, complete or substantially complete mucosal healing). Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. For purposes of this invention, beneficial or desired clinical results include, but are not limited to, one or more of the following: reducing, decreasing symptoms resulting from the disease, increasing the quality of life of those suffering from the disease, decreasing the dose of other medications required to treat the disease, enhancing the effect of another medication, delaying the progression of the disease, and/or prolonging survival of individuals. Treatment includes preventing the disease, that is, causing the clinical symptoms of the disease not to develop by administration of a protective composition prior to the induction of the disease, suppressing the disease, that is, causing the clinical symptoms of the disease not to develop by administration of a protective composition after the inductive event but prior to the clinical appearance or reappearance of the disease, inhibiting the disease, that is, arresting the development of clinical symptoms by administration of a protective composition after their initial appearance, preventing re-occurring of the disease and/or relieving the disease, that is, causing the regression of clinical symptoms by administration of a protective composition after their initial appearance.

The terms “subject,” “individual” or “patient” are used interchangeably herein, and refer to a vertebrate, for example a mammal, including a human. Mammals include, but are not limited to, murines, simians, humans, farm animals, sport animals, and pets. Tissues, cells and their progeny of a biological entity obtained in vitro or cultured in vitro are also encompassed.

“Signal transduction” is a process during which stimulatory or inhibitory signals are transmitted into and within a cell to elicit an intracellular response. A modulator of a signal transduction pathway refers to a compound, which modulates the activity of one, or more cellular proteins mapped to the same specific signal transduction pathway. A modulator may augment (agonist) or suppress (antagonist) the activity of a signaling molecule.

The term “selective inhibition” or “selectively inhibit” as referred to a biologically active agent refers to the agent's ability to preferentially reduce the target signaling activity as compared to off-target signaling activity, via direct or interact interaction with the target.

A “therapeutic effect” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “susceptibility” or “susceptible” as used herein, refers to a subject determined to be at risk for having a disease condition. Such a determination may be based on an analysis including, but not limited to, (i) familial disease history, (ii) a genotypic characteristic of the subject, and/or (iii) a phenotypic characteristic of the subject.

The term “co-administration,” “administered in combination with,” and their grammatical equivalents, as used herein, encompasses administration of two or more agents to a subject so that both agents and/or their metabolites are present in the subject at the same time. Co-administration includes simultaneous administration in separate compositions, administration at different times in separate compositions, or administration in a composition in which both agents are present.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “pharmaceutically acceptable prodrug” as used herein refers to those prodrugs of the compounds of the present invention which are, within the scope of sound medical judgement, suitable for use in contact with the tissues of humans and lower animals with undue toxicity, irritation, allergic response, and the like, commensurate with a reasonable benefit/risk ratio, and effective for their intended use. The term “prodrug” refers to compounds that are rapidly transformed in vivo to yield the parent compound of the above formula, for example by hydrolysis in blood. Functional groups that may be rapidly transformed, by metabolic cleavage, in vivo form a class of groups reactive with a hydroxyl group of the compounds of this invention that may be acylated. Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

### EMBODIMENTS

### Composition Embodiments

In some embodiments, a beta blocker is selected from the group consisting of nadolol, timolol, harmalol, levobunolol, bisoprolol, alprenolol, carteolol, pindolol, metoprolol, acebutolol, S-propranolol, (−)-atenolol, sotalol, propranolol, R-atenolol, penbutolol, labetalol, pronetalol, oxprenolol, practolol, betaxolol, and dexpropranolol.

In other embodiments, a beta blocker is a compound having the structure:

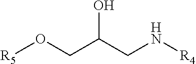

wherein R₄ is H or optionally substituted C₁-C₆ alkyl; R₅ is optionally substituted aryl, substituted heteroaryl, or optionally substituted cycloalkyl; or a pharmaceutically acceptable salt or a pharmaceutically acceptable prodrug thereof. In some embodiments, R₄ is C₁-C₆ alkyl for instance, R₄ can be iso-propyl or tert-butyl. In some embodiments, R₅ is optionally substituted aryl or optionally substituted heteroaryl. In some embodiments, R₅ is optionally substituted phenyl, indolyl, naphthyl, thiadiazyl. In some embodiments, R₅ is aryl optionally substituted with one or more substituents chosen from the group consisting of OH, cyano, C₁-C₆ heterocyclo, R₆, —OR₆, —CONH₂, —NHC(O)R₆, —R₆—OR₆, and —R₆—O—R₆—OR₆; wherein each R₆ is independently C₁-C₆ alkyl or C₁-C₆ cycloalkyl.

Some embodiments of the present invention comprise administering to a subject a compound of Formula I or a pharmaceutically acceptable salt thereof:

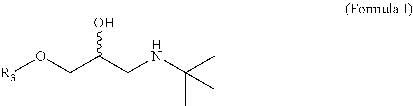

wherein R₃ is

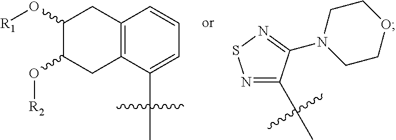

and R₁ and R₂ are independently H or C₁-C₆ alkyl.

In one embodiment, the compound of Formula I is:

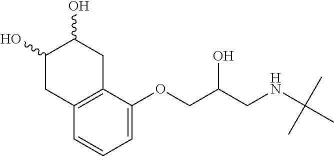 [SMILES: CC(C)(C)NCC(O)COc1cccc2c1CC(O)C(O)C2]

or a pharmaceutically acceptable salt thereof.

In some embodiments, the compound of Formula I is:

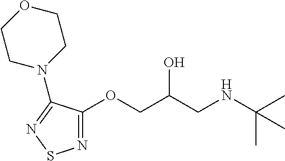 [SMILES: CC(C)(C)NCC(O)COc1nsnc1N1CCOCC1]

or a pharmaceutically acceptable salt thereof.

In some embodiments, the compound of Formula I is:

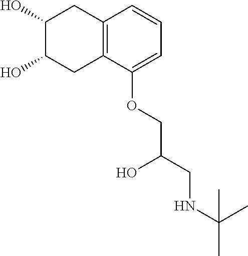 [SMILES: CC(C)(C)NCC(O)COc1cccc2c1C[C@H](O)[C@H](O)C2]

or a pharmaceutically acceptable salt thereof.

The compounds presented herein with stereocenters include all diastereomeric, enantiomeric, and epimeric forms as well as the appropriate mixtures thereof. Further, included are compositions in which such compounds are in a racemic mixture or in a composition having an enantiomeric excess of a particular stereoisomer. Purified stereoisomers are obtained, if desired, by methods such as, for example, the separation of stereoisomers by chiral chromatographic columns.

The methods and formulations described herein include the use of N-oxides, crystalline forms (also known as polymorphs), or pharmaceutically acceptable salts of the compounds of the invention, as well as active metabolites of these compounds having the same type of activity. In addition, the compounds described herein exist in unsolvated as well as solvated forms with pharmaceutically acceptable solvents such as water, ethanol, and the like. The solvated forms of the compounds presented herein are also considered to be disclosed herein.

Further provided herein are pharmaceutically acceptable compositions comprising a compound of Formula I as defined herein.

For example, the composition is formulated for administration via pH-dependent release delivery, microbially-triggered delivery, time-controlled delivery, osmotically-regulated delivery, pressure-controlled delivery, multi matrix systems delivery, bioadhesion delivery, or multiparticulate delivery. The composition can also be formulated for release in the small or large intestine, colon, rectum, stomach, anus, or esophagus. For example, the composition is formulated for release in the rectum.

In some embodiments, the compositions disclosed herein may be delivered to any part of the digestive tract, including but not limited to the colon, the intestine, the stomach, or the esophagus. For example, a composition is delivered primarily to the colon via a colon-specific drug delivery method. Possible colon-specific drug delivery methods include, but are not limited to, the following: pH-dependent release, microbially-triggered drug delivery, conjugates, time-controlled delivery, osmotically-regulated delivery, pressure-controlled delivery, multi matrix delivery systems, bioadhesion, microparticulate or nanoparticulate delivery. The drug delivery method may include any combination of colon-specific drug delivery methods. For example, a compound may be formulated to minimize release of the drug in the stomach, small intestine, and/or upper GI tract and to promote release of the drug in the colon. The route of administration may be topical, enteral, parenteral, or a combination thereof. Enteral administration routes comprise a non-local administration of the drug via the digestive tract. Common enteral routes include, but are not limited to: oral, rectal, sublingual, sublabial, buccal, gastric feeding tube, duodenal feeding tube, or a combination thereof. Topical routes include local administration of the substance directly onto the affected area. Parenteral routes include administrative via routes other than the digestive tract.

The compound may be coated, in some embodiments, with a pH-dependent polymer that inhibits or minimizes release of the drug in the stomach, small intestine, and/or upper GI tract and promotes release of the drug in the colon. The drug core may include tablets, capsules, pellets, granules, microparticles, nanoparticles, or a combination thereof. A suitable pH-dependent polymer may be a derivative of acrylic acid or cellulose. Examples of suitable pH-dependent polymers include, but are not limited to: hydroxypropyl methylcellulose phthalate, hydroxypropyl methylcellulose acetate succinate, acrylic polymers, acrylic copolymers, methyl methacrylate-methacrylic acid copolymers, cellulose acetate phthalate, cellulose acetate succinate, cellulose acetate trimellate, Eudragit® L100, Eudragit® L100-55, Eudragit® 5100, Eudragit® L-30D, Eudragit® FS 30D, hydroxypropyl methylcellulose phthalate 50, hydroxypropyl methylcellulose phthalate 55, or any combination thereof.

The drug compound may be embedded in a polymer matrix, wherein the polymer is pH-dependent. The drug delivery system may comprise Eduracol® technology, wherein a drug core is enclosed by multiple layers of Eudragit®. The drug delivery may comprise enclosure of a drug compound by multiple layers of pH-dependent polymers. The timing of the drug release may be controlled by the thickness and composition of the multiple layers surrounding the drug core.

The drug compound may be formulated to comprise a biodegradable polymer that reacts with the microflora or enzymes present in the colon, thereby facilitating delivery of the drug to the colon and inhibiting release of the drug in the stomach or small intestine. The drug compound may be coated with a biodegradable polymer. The drug compound may be embedded in biodegradable polymeric matrices or hydrogels. The drug delivery system may comprise both biodegradable polymers and pH-dependent polymers. The biodegradable polymer may be selected to react with a targeted colonic bacteria or enzyme. Examples of enzymes that a biodegradable polymer may react with include, but are not limited to: azoreductase, bacterial hydrolase glycosidase, esterase, polysaccharidase, and the like. For example, biodegradable polymers containing azoaromatic linkages may react with azoreductase. Examples of suitable biodegradable polymers include, but are not limited to: azo polymers, polyurethane containing azo aromatic groups, azo polymers using styrene-2-hydroxyethyl methacrylate and divinyl azobenzene as a cross linker, copolymers of 2-hydroxyethyl methacrylate with bis(methacryloylamino)azo benzene as a cross linker, terapolymers of methyl methacrylate, 2-hydroxyethyl methacrylate and methacrylic acid with N,N-bis[(methacryloyloxyethyl)oxy(carbonylamino)]azo benzene, divinyl azobenzene, and bis(methacryloylamino)azobenzene as a cross linker, poly(ether-ester) azopolymers, urethanes containing azo aromatic linkages in the backbone, polygalactomannans in polymethacrylate solutions, inulin and copolymers of acrylic acid esters, pectin and ethyl cellulose, glutaraldehyde cross-linked dextran, poly(-caprolactone), polylactic acid, poly(lactic-co-glycolic acid), polysaccharides, amylose, guar gum, pectin, chitosan, inulin, cyclodextrin, chondroitin sulphate, dextran, locust bean gum, arabinogalactan, chondroitin sulfate, xylan, calcium pectinate, pectin/chitosan mixtures, amidated pectin, or any combination thereof.

In addition, a formulation of the drug compound may comprise a biodegradable matrix, which comprises cross-linked biodegradable polymers. The biodegradable polymers may be cross-linked to each other and added to the drug compound or the formulation. The biodegradable polymer matrix may comprise a synthetic hydrogel. The biodegradable polymer matrix may comprise a synthetic hydrogel based on N-substituted acrylamide. Additional hydrogels include, but are not limited to, hydrogels synthesized by cross-linking N-tert-butylacrylamide with acrylic acid or copolymerization of 2-hydroxyethyl methacrylate and 4-methacryloyloxyazobenzene.

A conjugate of the drug compound may be used to facilitate delivery of the drug compound to the colon. The conjugate may be formed by covalently linking the compound to a carrier molecule. The conjugate remains predominately intact in the stomach and small intestine. The covalent link is broken in the colon via an enzymatic or pH-related mechanism, thereby releasing the compound. Suitable linkages between the drug compound and the carrier molecule may include, but are not limited to, azo linkages, amide linkages, glycosidic linkages, and glucuronide linkages. Suitable conjugates include, but are not limited to: azo conjugates, cyclodextrin conjugates, glycoside conjugates, glucoronate conjugates, dextran conjugates, polypeptide conjugates, and polymeric conjugates. Examples of carrier molecules that may be linked to the compound via an aza linkage include, but are not limited to: 4-amino benzoyl glycine, sulfapyridine, 4-amino benzoyl-β-alanine, p-aminobenzoate, or sulphanilamide ethylene polymer. The conjugate may also be a dimer of the drug compound, wherein the compounds are connected via an azo linkage. Additional examples of polymeric carrier molecules that may be linked to the compound include, but are not limited to: polysulfonamidoethylene, poly(methyl vinyl ether/co-maleic anhydride), poly (1-vinyl-2-pyrrolidone co-maleic anhydride, chloroformate-activate derivates of dextran, poly [(2-hydroxyethyl)aspartamine], or a combination thereof.

The drug compound may be delivered via a time-controlled delivery system. An example of a suitable time-controlled delivery system is the Pulsincap® system, or a variant thereof. The time-controlled delivery system may further comprise pH-dependent systems, microbially-triggered delivery systems, or a combination thereof. The time-controlled system may comprise a water insoluble capsule body enclosing a drug reservoir. The capsule body may be closed at one end with a hydrogel plug. The hydrogel plug may comprise swellable polymers, erodible compressed polymers, congealed melted polymers, enzymatically controlled erodible polymers, or a combination thereof. The swellable polymers may include, but are not limited to, polymethacrylates. The erodible compressed polymers may include, but are not limited to, hydroxypropyl methylcellulose, polyvinyl alcohol, polyvinyl acetate, polyethylene oxide, or a combination thereof. The congealed melted polymers may include, but are not limited to: saturated polyglycolated glycerides, glyceryl monooleate, or a combination thereof. The enzymatically controlled erodible polymers may include, but are not limited to polysaccharides, amylose, guar gum, pectin, chitosan, inulin, cyclodextrin, chondroitin sulphate, dextrans, locust bean gum, arabinogalactan, chondroitin sulfate, xylan, calcium pectinate, pectin/chitosan mixtures, amidated pectin, or any combination thereof.

An additional example of a suitable time-controlled delivery system is Time Clock®, or a variant thereof, wherein a solid dosage form is coated with a hydrophobic surfactant layer to which a water-soluble polymer is added.

The time-controlled delivery system may comprise a capsule, which further comprises an organic acid. The organic acid may be filled into the body of a hard gelatine capsule. The capsule may be coated with multiple layers of polymers. The capsule may be coated first with an acid soluble polymer, such as Eudragit® E, then with a hydrophilic polymer, such as hydroxypropyl methylcellulose, and finally with an enteric coating, such as Eudragit® L.

An additional example of a suitable time-controlled delivery system is the Chronotropic® system, or a variant thereof, which comprises a drug core that is coated with hydroxypropyl methylcellulose and an outer enteric film.

An additional example of a suitable time-controlled delivery system is the CODES™ system, or a variant thereof. The time-controlled delivery system may comprise a capsule body, which may house a drug-containing tablet, an erodible tablet, a swelling expulsion excipient, or any combination thereof. The capsule may comprise an ethyl cellulose coat. The time-controlled delivery system may comprise two different sized capsules, one inside of the other. The space between the capsules may comprise a hydrophilic polymer. The drug-containing core may be housed within the inner capsule. The drug delivery system may comprise an impermeable shell, a drug-containing core, and erodible outer layers at each open end. When the outer layers erode, the drug is released.

The compound may be delivered to the colon via an osmotically-regulated delivery system. An example of a suitable osmotically-regulated delivery system is the OROS® Push-Pull osmotic pump technology, or a variant thereof. The osmotically controlled drug delivery system may comprise a semipermeable coat. The system may further comprise a bi-layer core containing two component layers within a compartment formed by a semipermeable wall having an exit means for release of the drug. In the bi-layer core, one layer contains the drug compound and the second layer contains osmotically active compounds. The two layers may be compressed into bi-layer tablet cores before the semipermeable membrane and exit means are applied. The bi-layer tablet cores may be longitudinally compressed with a different layer at each end. Osmotically active components include, but are not limited to, osmopolymers, which are polymers that have relatively large molecular weights and swell when exposed to fluid. The layer containing the osmotically active components may comprise one or more of the following compounds:
poly(ethylene)oxide, sodium chloride, poly(vinylpyrrolidone), red ferric oxide, magnesium stearate, butylated hydroxyl toluene, and hydroxypropyl methylcellulose. The semipermeable membrane may comprise cellulose acetate, poly(ethylene glycol), or a combination thereof.

Alternatively, the osmotically-regulated drug delivery system may comprise a tri-layer core containing three component layers within a compartment formed by a semipermeable membrane and comprising an exit means for release of the drug. In the tri-layer core, the first and second layers contain the drug compound, the third layer comprises the osmotically active components. The first and second layers may further comprise one or more of the following compounds: poly(ethylene)oxide, sodium chloride, poly(vinylpyrrolidone), red ferric oxide, magnesium stearate, butylated hydroxyl toluene, and hydroxypropyl methylcellulose. The third layer comprises osmopolymers. The semipermeable membrane may comprise cellulose acetate, poly(ethylene glycol), or a combination thereof.

An additional example of a suitable osmotically-regulated delivery system is the PORT® system, or a variant thereof. The osmotically-regulated drug delivery system may comprise a gelatin capsule coated with a semipermeable membrane housing an insoluble plug, an osmotically active agent, and the drug compound. The semipermeable membrane may comprise cellulose acetate, poly(ethylene glycol), or a combination thereof. The osmotically-regulated drug delivery system may comprise a water-insoluble capsule shell, which houses a drug containing compartment, an osmotic core, a rigid barrier layer, a semi-permeable cap, an insoluble coat on the capsule, or any combination thereof.

The pressure-controlled delivery system may comprise a capsule that is fabricated from a water insoluble material, such as ethyl cellulose. Suitable pressure controlled colon-delivery capsules withstand the luminal pressures of the stomach and small intestine, but release the drug under the pressurized conditions in the colon. The capsule may comprise two coatings. The capsule may comprise an insoluble coating on the inside wall of the capsule. The capsule may comprise an insoluble waxy plug that seals the top of the insoluble coat. The capsule may be coated with an enteric coating.

The drug delivery system may comprise the drug compound inglobulated in multiple matrices. An example of a suitable multi matrix delivery system is the MMX® delivery system (Cosmo Pharmaceuticals), or a variant thereof. The matrices may include, but are not limited to, amphiphilic, lipophilic and hydrophilic matrices. The multi matrix may be coated with a pH-resistant coating, biodegradable polymeric coating, or some combination thereof. The amphiphilic matrix may comprise polar lipids of type I or II, lecithin, phosphatidylcholine, phosphatidylethanolamine, polyethylene glycols, ceramides, glycol alkyl ether, diethylene glycol monomethyl ether, or a combination thereof. The lipophilic matrix may comprise C8-C20 fatty acids with glycerol, unsaturated or hydrogenated alcohols or fatty acids, salts, esters or amide derivatives thereof, waxes, ceramides, cholesterol derivatives, or a combination thereof. The hydrophilic matrix may comprise hydrophilic water-swellable agents, known as hydrogels. Suitable hydrogels include, but are not limited to, hydroxypropylcellulose, acrylic or methacrylic acid polymers or copolymers, alkylvinyl polymers, hydroxyalkyl celluloses, caroxyalkyl celluloses, polysaccharides, dextrins, pectins, starches and derivatives, natural or synthetic gums, alginic acid, or some combination thereof. The pH-resistant coating may comprise acrylic copolymers, methacrylic acid polymers or copolymers, cellulose derivatives, or a combination thereof.

In some multi matrix systems, the drug compound is embedded in biodegradable matrices. In some multi matrix systems, the drug compound is embedded in pH-dependent matrices. In some multi matrix systems, the drug compound is embedded in pH-dependent and biodegradable matrices.

The drug delivery system may comprise a bioadhesive polymer that selectively adheres to the colonic mucosa. Bioadhesive polymers facilitate extended contact of the drug with the tissues of a subject. The high molecular weight of these polymers readily swell in body fluids, providing a large adhesive surface for maximum contact with the mucous layer of such tissues as the colon. These polymers have been shown to increase drug bioavailability due to the lengthened period of time in which the drug is in contact with the absorbing tissue. Bioadhesive systems may comprise polymers such as polycarbophils, polyurethanes, polyethylene oxide-polypropylene oxide copolymers, or some combination thereof. Bioadhesive that could be used with Formula I include but are not limited to, Carbopol® polymers, Pemulen™ polymeric emulsifiers and Noveon® polycarbophils or variants thereof.

Examples of suitable multiparticulate drug delivery systems include Diffucaps®, Diffutab®, Orbexa®, Eurand Minitabs®, Microcaps®, or a variant thereof. The drug delivery system may comprise multiparticulate beads, which are comprised of multiple layers of the drug compound, excipients, and release-controlling polymers. The multiparticulate beads may comprise an organic acid or alkaline buffer. The multiparticulate beads may comprise a solid solution of the drug compound and crystallization inhibitor. The drug delivery system may comprise a matrix tablet containing water-soluble particles and the drug compound. The matrix tablet may further comprise hydrophilic and hydrophobic polymers. In some multiparticulate delivery systems, particles in the micron size range are used. In some multiparticulate delivery systems, nanoparticle colloidal carriers composed of natural or synthetic polymers are used.

Therapeutic agents used to practice the methods of the invention, including compositions comprising compounds of Formula I, can dissolve or disintegrate rapidly in a subject's mouth when administered orally. Therefore, a pleasant taste can be important for ensuring compliance with a therapeutic regimen. Taste-masking technologies provide the benefit of an agreeable taste and an absence of grit residue left behind in the mouth, thus leaving the subject with a good mouth feel. Examples of suitable taste-masking technologies that can be used with Formula I include, but are not limited to: Zydis®, Quicksolv®, Lyoc®, NanoCrystal®, AdvaTab®, OraSolv®, DuraSlov®, FlashDose®, Flash Melt®, Flashtab®, OraQuick®, Pharmburst®, Frosta® and WOWTAB®.

Therapeutically-effective dosages vary in some embodiments when the compounds of the invention are used in treatment combinations. Methods for experimentally determining therapeutically-effective dosages of drugs and other agents for use in combination treatment regimens include the use of metronomic dosing, i.e., by providing more frequent, lower doses in order to minimize toxic side effects. Combination treatment regimens encompass treatment regimens in which administration of a compound described herein is initiated prior to, during, or after treatment with a second agent described above, and continues until any time during treatment with the second agent or after termination of treatment with the second agent. Such regimens also include treatments in which a compound described herein and the second agent being used in combination are administered simultaneously or at different times and/or at decreasing or increasing intervals during the treatment period. Combination treatments further include periodic treatments that start and stop at various times to assist with the clinical management of the patient. For example, a compound described herein in the combination treatment is administered weekly at the onset of treatment, decreasing to biweekly, and decreasing further as appropriate.

In any combination treatment regimen, a therapeutically effective amount of the compound of the invention is optionally administered. Alternatively, the compound of the invention is administered in an amount which is less therapeutically effective if administered alone. For any of the classes of additional therapeutic agents mentioned herein that are used in combination treatments, the additional therapeutic agent is optionally administered in an amount which would be less therapeutically effective if administered alone. By way of example only, if an analgesic is used in a combination treatment, the analgesic is administered in an amount which is less therapeutically effective if administered alone.

In any case, the therapeutic agents that make up a combination treatment (at least one of which is a compound of the invention) are administered in any order or even simultaneously. If administered simultaneously, the multiple therapeutic agents are optionally provided in a single, unified form, or in multiple forms (by way of example only, either as a single pill or as two separate pills). One of the therapeutic agents is optionally given in multiple doses, or both are given as multiple doses. If not simultaneous, the timing between the multiple doses optionally varies from more than zero weeks to less than four weeks. In addition, the combination methods, compositions and formulations are not to be limited to the use of only two agents, the use of multiple therapeutic combinations are also envisioned.

In addition, the compounds of the invention are also used in combination with procedures that provide additional or synergistic benefit to the patient. By way of example only, patients are expected to find therapeutic and/or prophylactic benefit in the methods described herein, wherein pharmaceutical composition of the invention and/or combinations with other therapeutics are combined with genetic testing to determine whether that individual is a carrier of a mutant gene that is known to be correlated with certain diseases or conditions.

The compounds of the invention and combination therapies according to the invention are administered before, during and/or after the occurrence of a disease or condition, and the timing of administering the composition containing a compound optionally varies. Thus, for example, the compounds are used as a prophylactic and are administered continuously to subjects with a propensity to develop conditions or diseases in order to prevent the occurrence of the disease or condition. The compounds and compositions are optionally administered to a subject during or as soon as possible after the onset of the symptoms. For example, the administration of the compounds is initiated within the first 48 hours of the onset of the symptoms, for example within the first 48 hours of the onset of the symptoms, or within the first 6 hours of the onset of the symptoms, or within 3 hours of the onset of the symptoms. The initial administration is via any route practical, such as, for example, an intravenous injection, a bolus injection, infusion over 5 minutes to about 5 hours, a pill, a capsule, transdermal patch, buccal delivery, and the like, or combination thereof. A compound is administered, for example, as soon as is practicable after the onset of a disease or condition is detected or suspected, and for a length of time necessary for the treatment of the disease, such as, for example, from about 1 month to about 3 months. The length of treatment potentially varies for each subject, but the length is determined using the known criteria. For example, the compound or a formulation containing the compound is administered for at least 2 weeks, about 1 month to about 5 years, or from about 1 month to about 3 years.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in some embodiments, the drug is delivered in a targeted drug delivery system, for example, in a liposome coated with organ-specific antibody. In such embodiments, the liposomes are targeted to and taken up selectively by the organ. In some embodiments, the compound as described herein is provided in the form of a rapid release formulation, in the form of an extended release formulation, or in the form of an intermediate release formulation. In some embodiments, the compound described herein is administered topically.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins1999).

Provided herein are pharmaceutical compositions comprising a compound of Formula I, and a pharmaceutically acceptable diluent(s), excipient(s), or carrier(s). In certain embodiments, the compounds described are administered as pharmaceutical compositions in which compounds of Formula I, are mixed with other active ingredients, as in combination therapy. Encompassed herein are all combinations of actives set forth in the combination therapies section below and throughout this disclosure. In specific embodiments, the pharmaceutical compositions include one or more compounds of Formula I.

A pharmaceutical composition, as used herein, refers to a mixture of a compound of Formula I, with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Formula I, provided herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In some embodiments, one or more compounds of Formula I, is formulated in an aqueous solutions. In specific embodiments, the aqueous solution is selected from, by way of example only, a physiologically compatible buffer, such as Hank's solution, Ringer's solution, or physiological saline buffer. In some embodiments, one or more compound of Formula I, is formulated for transmucosal administration. In specific embodiments, transmucosal formulations include penetrants that are appropriate to the barrier to be permeated. In some embodiments wherein the compounds described herein are formulated for other parenteral injections, appropriate formulations include aqueous or nonaqueous solutions. In specific embodiments, such solutions include physiologically compatible buffers and/or excipients.

In some embodiments, compounds described herein are formulated for oral administration. For clarity, formulations intended for colonic delivery can be administered by oral administration. Compounds described herein, including compounds of Formula I, are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose, or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In some embodiments, dosage forms, such as dragee cores and tablets, are provided with one or more suitable coating. In specific embodiments, concentrated sugar solutions are used for coating the dosage form. The sugar solutions optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In some embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In some embodiments, therapeutically effective amounts of at least one of the compounds described herein are formulated for buccal or sublingual administration. Formulations suitable for buccal or sublingual administration include, by way of example only, tablets, lozenges, or gels. In some embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In some embodiments, the pharmaceutical composition of Formula I, are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in some embodiments, the active ingredient is in powder form for constitution with a suitable vehicle, e.g., sterile pyrogen-free water, before use.

In some embodiments, the compounds of Formula I are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In some embodiments, the compounds of Formula I are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Formula I is accomplished by means of iontophoretic patches and the like. In certain embodiments, transdermal patches provide controlled delivery of the compounds of Formula I. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in some embodiments, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In some embodiments, the compounds of Formula I are formulated for administration by inhalation. Various forms suitable for administration by inhalation include, but are not limited to, aerosols, mists or powders. Pharmaceutical compositions of Formula I, are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In some embodiments, the compounds of Formula I are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

For example, a compounds of Formula I (e.g., in powder form) is typically dispersed in a suppository base, such as hard fat. The suppository base can be an oily or fatty base. Conventional suppository bases which may be employed include theobroma oil, hard fats, glycerides of fatty acids, glycerol-gelatin bases, and mixtures thereof. Suitable hard fat bases include, but are not limited to, esterified mixtures of mono-, di- and triglycerides which are obtained by esterification of fatty acids (European Pharmacopoeia, 3rd edition 1997, Deutscher Apotheker Verlag Stuttgart. p. 1022; The United States Pharmacopoeia, USP 23, NF18). Such hard fats are commercially available, for example, under the name Witepsol® (e.g. Witepsol® H12 and H15). A preferred suppository base is hard fat (e.g., hard fat NF). Preferred hard fat bases include, but are not limited to, hard fats containing a mixture of mono-, di- and triglycerides of saturated C₉₋₁₈ fatty acids. The hard fat base can comprise hard fats obtained by esterification of fatty acids of vegetable origin with glycerol, a macrogol ether containing 20 to 24 oxyethylene groups in the polyoxyethylene chain, e.g., polyoxyl-20-cetostearyl ether, and glycerides, e.g., glyceryl ricinoleate. Other suitable suppository bases include, but are not limited to, cocoa butter, lauric oil, beef tallow, hard fat, and any combination of any of the foregoing. The drug load of the suppository is, for example, between 30 and 60%, for example 35 to 50% or 37% to 50%. According to one embodiment, the drug load ranges from about 37 to about 46%. According to some embodiments, the drug load ranges from about 39 to about 45%. According to some embodiments, the drug load ranges from about 41 to about 43%. For example, the suppository can contain about 1000 mg compound of Formula I dispersed in about 1300 to about 1500 mg of a suppository base (preferably hard fat). In some embodiments, the total weight of the suppository ranges from about 500 to 5000 mg, from 1000 to 4000 mg, from 1500 to 3500 mg, from 2250 to about 2700 mg or from about 2250 to about 2500 mg. The suppository is preferably smooth torpedo-shaped. The melting point of the suppository is generally sufficient to melt in the patient's body, and is typically no more than about 37° C.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Formula I, are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Formula I, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances. Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of Formula I, illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In some embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also optionally include solubilizing agents to aid in the solubility of a compound of Formula I. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

Additionally, useful compositions also optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal, stabilized chlorine dioxide, and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc, or (n) combinations thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein might be employed in practicing the invention. It is intended that the following claims define the scope of the invention and that methods and structures within the scope of these claims and their equivalents be covered thereby.

Modulation of gene and signaling pathways embodiments

In some aspects, provided herein are novel therapeutic methods and compositions and the uses of these agents for treatment of diseases mediated by particular pathways and genes.

In some embodiments, compounds of Formula I have one or more effects in a cell or in a subject including, but not limited to, anti-inflammatory, immunosuppressive and other immunodulatory effects. In eliciting these effects, compounds of Formula I modulate one or more genes and signal transduction pathways or networks in the cell or body of the subject.

In some embodiments, compounds of Formula I modulate a signal transduction pathway including, but not limited to, a TREM1 signaling pathway, an antigen presentation pathways, a TGF-β pathway, a T-cell or B-cell pathway, a toll-like receptor pathway, a complement system pathway, a leukocyte extravasation pathway, a T helper (Tₕ) cell differentiation pathway, a graft-versus-host disease pathway, an autoimmune thyroid disease pathway, a macrophage pathway, a fibroblast or endothelial cell pathway (e.g. a pathway involved in rheumatoid arthritis), an IL-12 signaling and/or production pathway, a cytokine-mediated communication pathway (e.g. between immune cells), a dendritic cell maturation pathway, a systemic lupus erythematosus pathway, a B-cell development pathway, and an airway inflammation (asthma) pathway.

In some embodiments, compounds of Formula I modulate one or more pathway, including, but not limited to, canonical pathways that are relevant to inflammatory bowel disease and/or autoimmune disease. Non-limiting examples of pathways which are modulated by compounds of Formula I include pathways for: crosstalk between dendritic cells and natural killer cells, cytokine mediated communication between immune cells, communication between innate and adaptive immune cells, differential regulation of cytokine production in intestinal epithelial cells by IL-1A and IL-17F, altered T-cell and B-cell signaling in rheumatoid arthritis, leukocyte extravasation signaling, complement system, LPS/IL-1 mediated inhibition of RXR function, differential regulation of cytokine production in macrophages and T helper cells by IL-17A and IL-17F, and/or dendritic cell maturation.

In some embodiments, the compound of Formula I modulates the activity of the TREM1 pathway. TREM1 belongs to the Immunoglobulin (Ig) family of cell surface receptors and is selectively expressed on blood neutrophils, monocytes and macrophages. TREM1 lacks known signaling motifs in the cytoplasmic domain and thus activation by TREM1 is believed to be mediated by a transmembrane adaptor molecule DNAX-activating protein 12 (DAP12), leading to proinflammatory immune responses. The natural ligand for TREM1 is unknown. TREM1 activation triggers the Janus kinase 2 (JAK2), protein kinase B (PKB/AKT) and extracellular signal related kinase (ERK1/2) pathways leading to the phosphorylation of signal transducers of activation of transcription (STAT3,5) and NF kappa B (NF-kB). These transcription factors upregulate the expression of genes involved in the inflammatory response. Stimulation of TREM1 by its ligand or toll like receptor (TLR) by lipopolysaccharide (LPS) can lead to an association of TREM1 and TLR. This association leads to the activation of interleukin-1 receptor-associated kinase 1(IRAK1), which in turn triggers NF-kB and the proinflammatory response. Engagement and activation of TREM1 triggers expression and secretion of chemokines and cytokines like monocyte chemotactic protein 1(MCP-1), macrophage inflammatory protein-lalpha (MIP-1a), interleukins (IL-6,-8) and tumor necrosis factor (TNF). Many of these effects are potentiated by LPS. Cytokines like TNF and granulocyte macrophage colony stimulating factor (GM-CSF) in turn upregulate the expression of TREM1 in an autocrine fashion. The synergy between TLR and TREM1 leads to neutrophil degranulation, phagocytosis and the respiratory burst in addition to the production of proinflammatory cytokines. TREM1 activation also results in the upregulation of cell surface proteins such as CD11, CD29 and CD40, CD83 that are involved in cell adhesion and costimulation respectively, as well as phospholipase gamma (PLCg) mediated Ca²⁺ release. Thus TREM1 activation is involved in diverse aspects of innate and adaptive immune response. In addition to TLRs, TREM1 also synergizes with a second major class of pattern recognition receptors—the NACHT-LRR receptors (NLR), which recognize intracellular microorganisms. The TREM1/NLR synergism results in the production of proinflammatory cytokines like TNF, IL-1b, IL-6 and IL-18—the latter three via a caspase-1 dependent pathway. Thus TREM1 acts to amplify signals from both major pathways of pattern recognition—extracellular TLR receptors and the intracellular NLR proteins. By interacting with one or more components of the TREM1 signaling pathway, a compound of Formula I modulates the activity of this pathway.

In some embodiments, the compound of Formula I modulates the activity of an antigen presentation pathway. These pathways mediate a process in the body's immune system by which macrophages, dendritic cells and other cell types capture antigens and then enable their recognition by T-cells. Antigen presentation pathways include the endogenous pathway, which is used to present cellular peptide fragments on the cell surface on MHC class I molecules, and the exogenous pathway, which is utilized by specialized antigen presenting cells to present peptides derived from proteins that the cell has endocytosed, and which peptides are presented on MHC class II molecules. By interacting with one or more components of an antigen presentation pathway(s), a compound of Formula I modulates the activity of these pathways.

In some embodiments, a compound of Formula I modulates the activity of a TGF-β pathway. The TGF-β superfamily of ligands include: bone morphogenetic proteins (BMPs), growth and differentiation factors (GDFs), anti-Müllerian hormone (AMH), Activin, Nodal and TGFβ's. Signaling is believed to begin with the binding of a TGF-β superfamily ligand to a TGF-β type II receptor. The type II receptor is a serine/threonine receptor kinase, which catalyzes the phosphorylation of the Type I receptor. Each class of ligand is believed to bind to a specific type II receptor. Mammals have seven known type I receptors, five type II receptors. Three activins are known, and include Activin A, Activin B and Activin AB. Activins are involved in embryogenesis and osteogenesis, and regulate hormones including, but not limited to pituitary, gonadal and hypothalamic hormones, as well as insulin. Activins also act as nerve cell survival factors. BMPs bind to the Bone morphogenetic protein receptor type-2 (BMPR2), and are involved in a multitude of cellular functions including osteogenesis, cell differentiation, anterior/posterior axis specification, growth, and homeostasis. Nodal is involved in cell differentiation, and is believed to be involved in mesoderm formation and subsequent organization of left-right axial structures in early embryonic development. The TGF-β family further includes ligands such as TGFβ1, TGFβ2, and TGFβ3. By interacting with one or more components of a TGF-β pathway, a compound of Formula I modulates the activity of this pathway.

In some embodiments, a compound of Formula I modulates the activity of a T-cell or B-cell pathway. Such pathways are, for example, involved in rheumatoid arthritis (RA). RA is a form of autoimmunity, the causes of which are still incompletely known, but is believed to involve abnormal B cell-T cell interaction. Inflammation can be driven either by B cell or T cell products, stimulating release of TNF and other cytokines. By interacting with one or more components of a T-cell or B-cell pathway, a compound of Formula I modulates the activity of this pathway.

In some embodiments, a compound of Formula I modulates the activity of a Toll-like receptor pathway. Toll-like receptors (TLRs) recognize distinct pathogen-associated molecular patterns and play a critical role in innate immune responses. They participate in the first line of defense against invading pathogens and play a significant role in inflammation, immune cell regulation, survival, and proliferation. A number of the TLR family have been identified, of which TLR1, TLR2, TLR4, TLR5, and TLR6 are located on the cell surface and TLR3, TLR7, TLR8, and TLR9 are localized to the endosomal/lysosomal compartment. The activation of the TLR signaling pathway originates from the cytoplasmic Toll/IL-1 receptor (TIR) domain that associates with a TIR domain-containing adaptor, MyD88. Upon stimulation with ligands, MyD88 recruits IL-1 receptor-associated kinase-4 (IRAK-4) to TLRs through interaction of the death domains of both molecules. IRAK-1 activated by phosphorylation then associates with TRAF6, finally leading to activation of MAP kinases (JNK, p38 MAPK) and NF-κB. Tollip and IRAK-M interact with IRAK-1 and negatively regulate the TLR-mediated signaling pathways. Additional modes of regulation for these pathways include TRIF-dependent induction of TRAF6 signaling by RIP1 and negative regulation of TIRAP mediated downstream signaling by ST2L, TRIAD3A, and SOCS1. MyD88-independent pathways induce activation of IRF3 and expression of interferon-ft TIR-domain containing adaptors such as TIRAP, TRIF, and TRAM regulate TLR-mediated signaling pathways by providing specificity for individual TLR signaling cascades. By interacting with one or more components of a Toll-like receptor pathway, a compound of Formula I modulates the activity of this pathway.

In some embodiments, a compound of Formula I modulates the activity of a complement system pathway. The complement system has four major function, including lysis of infectious organisms, activation of inflammation, opsonization and immune clearance. Complement pathways include the classical complement pathway, the alternative complement pathway, and the mannose-binding lectin pathway. The classic complement pathway is triggered when antibody-antigen complex interact with C1-complex, which consists of C1q, two molecules of C1r, and two molecules of C1s. The C1-complex cleaves C2 and C4, which then form C3 convertase (C4b2a). C3 is then cleaved by the C3 convertase, and forms C5 convertase in association with C4b and C2a. The generation of C5 convertase is the end of the classical pathway. The lectin pathway is similar to the classical pathway, and is stimulated when the mannose-binding lectin (MBL) binds to mannose residues on the pathogen surface. The MBL-associated serine proteases, MASP-1, and MASP-2, are activated and cleave C4 and C2, which then form the C3 convertase as in the classical pathway. The alternative complement pathway begins with the activation of C3 and requires factor B and factor D. All three pathways merge at C3, which is then converted into C3a and C3b. The further formed C5 convertase from C3b cleaves C5 into C5a and C5b. C5b with C6, C7, C8, and C9 complex to form the membrane attack complex (MAC), which is inserted into the cell membrane, forms a hole in the membrane, and initiates cells lysis. At least 12 proteins are known to be involved in regulation of the complement system, including Factor H, Factor I, C1 inhibitor, and CD59. Factor H removes Bb from the alternative pathway C3 convertase, breaking the positive feedback loop. Factor I inactivates C3b. C1 inhibitor binds to sites on activated C1r and C1s shutting down their proteolytic activity. CD59, also known as protectin, inhibits C9 polymerization during the formation of the membrane attack complex. By interacting with one or more components of a complement system pathway, a compound of Formula I modulates the activity of this pathway.

In some embodiments, a compound of Formula I modulates the activity of a leukocyte extravasation pathway. This pathway stimulates the movement of leukocytes out of the circulatory system, towards the site of tissue damage or infection. Upon recognition of and activation by pathogens, resident macrophages in the affected tissue release cytokines such as IL-1, TNFα and chemokines. IL-1 and TNFα cause the endothelial cells of blood vessels near the site of infection to express cellular adhesion molecules, including selectins and integrins. Circulating leukocytes are localized towards the site of injury or infection due to the presence of chemokines. By interacting with one or more components of a leukocyte extravasation pathway, a compound of Formula I modulates the activity of this pathway.

In some embodiments, a compound of Formula I modulates the activity of a T helper cell (Tₕ) differentiation pathway. Tₕ cell's progenitors differentiate into effector Tₕ cells, memory Tₕ cells, and regulatory Tₕ cells. Once two-signal activation is complete, Tₕ proliferates by releasing a T-cell growth factor called interleukin 2 (IL-2), which acts in an autocrine fashion. Activated T-cells also produce the alpha sub-unit of the IL-2 receptor (CD25 or IL-2R), enabling a fully functional receptor that can bind with IL-2, which in turn activates the T cell's proliferation pathways. The autocrine or paracrine secretion of IL-2 can bind that same Tₕ cell or neighboring Tₕ's via the IL-2R thus driving proliferation and clonal expansion. The Tₕ cells receiving both signals of activation will then become Tₕ0 cells (T helper 0) cell that secrete IL-2, IL-4 and interferon gamma (IFN-γ). The Tₕ0 cells will then differentiate into Tₕ1 or Tₕ2 cells depending on cytokine environment. IFN-γ drives Tₕ1 cell production while IL-10 and IL-4 inhibit Tₕl cell production. Conversely, IL-4 drives Tₕ2 cell production and IFN-γ inhibits Tₕ2 cells. Such cytokines are pleiotropic and carry out many other functions of the immune response. By interacting with one or more components of a Tₕ differentiation pathway, a compound of Formula I modulates the activity of this pathway.

In some embodiments, a compound of Formula I modulates the activity of a graft-versus-host disease (GVHD) signaling pathway. This pathway is involved in GVHD, which is a lethal complication of allogeneic hematopoietic stem cell transplantation (HSCT) where immunocompetent donor T-cells attack the genetically disparate host cells. GVHD pathophysiology involves a three-step process, where the first step is the development of an inflammatory milieu resulting from damage in the host tissues induced by the preparative chemotherapy or radiotherapy regimen. Damaged tissues secrete inflammatory cytokines, including interleukin 1 (IL-1), and tumor necrosis factor (TNF-alpha). During the second step, antigen-presenting cells (APCs) trigger the activation of donor-derived T cells, which induce further T-cell expansion, induce cytotoxic T lymphocytes (CTL) and natural killer (NK) cells responses and prime additional mononuclear phagocytes to produce TNF-alpha and IL-1. Also, nitric oxide (NO) is produced by activated macrophages, and it may contribute to the tissue damage observed subsequently. During the third step, the effector phase, activated CTL and NK cells mediate cytotoxicity against target host cells through Fas-Fas ligand interactions and perforin-granzyme B. By interacting with one or more components of the GVHD pathway, a compound of Formula I modulates the activity of this pathway.

In some embodiments, a compound of Formula I modulates the activity of an autoimmune thyroid disease pathway. This disease pathway includes, for example, Hashimoto's thyroiditis (HT) or chronic autoimmune thyroiditis and its variants, Graves' disease (GD) and autoimmune atrophic thyroiditis or primary myxedema. HT is characterized by the presence of goitre, thyroid autoantibodies against thyroid peroxidase (TPO) and thyroglobulin (Tg) in serum and varying degrees of thyroid dysfunction. During HT, self-reactive CD4+ T lymphocytes (Th) recruit B cells and CD8+ T cells (CTL) into the thyroid. Disease progression leads to the death of thyroid cells and hypothyroidism. Both autoantibodies and thyroid-specific cytotoxic T lymphocytes (CTLs) have been proposed to be responsible for autoimmune thyrocyte depletion. In Graves' disease, the thyroid stimulating hormone receptor (TSH-R) is the most important autoantigen. Antibodies directed against it mimic the effects of the hormone on thyroid cells, TSH, stimulating autonomous production of thyroxine and triiodothyronine and causing hyperthyroidism. The presence of TSH-R-blocking antibodies that bind the TSH receptor in a similar fashion to the antibodies in patients with Graves' disease but that block rather than activate the receptor explains some cases of atrophic hypothyroidism. By interacting with one or more components of an autoimmune thyroid disease pathway, a compound of Formula I modulates the activity of this pathway.

In some embodiments, a compound of Formula I modulates the activity of a pathway which controls or determines the function of macrophages, fibroblast and endothelial cells. Such pathways are important, for example, in disorders such as rheumatoid arthritis (RA). The pathology of RA is characterized by the infiltration of several inflammatory cells into both the pannus and the joint fluid, and by subsequent tissue destruction. Chemokines, as well as other inflammatory mediators play key roles in the pathogenesis of RA, and the coordinated production of chemokines and pro-inflammatory cytokines is important in the orchestration of the inflammatory responses observed in patients with RA. Monocytes that are attracted to the RA joint differentiate into macrophages and become activated. These macrophages play a pivotal role in RA because they are numerous in the inflamed synovial membrane and at the cartilage-pannus junction. They activate MHC Class-II (Major Histocompatibility Complex Class-II) molecules, and secrete proinflammatory or regulatory cytokines and growth factors like IL-1, IL-2, IL-6, IL-10, IL-13, IL-15, IL-17, IL-18, TNF-Alpha (Tumor Necrosis Factor), GMCSF (Granulocyte-Macrophage Colony-Stimulating Factor), chemokines and chemoattractants (e.g. IL-8, MIP1 (Macrophage Inflammatory Protein-1) and MCP1 (Monocyte Chemoattractant Protein), metalloproteinases and neopterin. TNF regulates IL-1β expression, which is important for the induction of prostanoid and MMP (Matrix Metalloproteinases) production by synovial fibroblasts and chondrocytes. Cellular interactions mediated by TNF and IL-1 cytokines that are mainly produced by activated macrophages are prominent factors leading to cartilage damage in RA. TNF increases the expression of adhesion molecules on endothelial cells, which recruit more cells to the joint. MCP1 and IL-8 are also secreted by macrophages and attract more cells into the joint. IL-1 and TNF induce synovial fibroblasts to express IL-6, chemokines, GMCSF and MMPs, which contribute to cartilage and bone destruction. TNF contributes to osteoclast activation and differentiation. In addition, IL-1 mediates cartilage degradation directly by inducing the expression of MMPs by chondrocytes. However, these cells of the innate immune system possess broad proinflammatory, destructive and remodeling capacities, and considerably contribute to inflammation and joint destruction both in the acute and chronic phases of RA. In addition, these chemokines produced by RA synovial stromal cells also stimulate monocyte migration. Other cytokines such as Ifn-Gamma (Interferon-Gamma) induced chemokines also contribute to the documented morphologic and clinical features of RA. The etiology of RA also involves abnormal presentation of self antigen(s) by APCs (Antigen Presenting Cells) and activation of autoreactive T-cells. T lymphocytes play a central role in the disease process. The rheumatoid synovial membrane is rich in MHC Class-II, APCs, and CD4+ T-Cells. APCs require signals from activated T-cells for their differentiation and maturation, this subsequently enables APCs to activate newly arrived T-cells in a specific or unspecific manner in the local inflammation. Activated T-cells promote the disease progression by inducing the secretion of pro-inflammatory cytokines (TNF-Alpha) from macrophages and synovial cells in a contact-dependent manner. Several costimulatory molecules are involved during APC-T-Cell interactions, including CD28/CD80-86 and CD40-CD40L. Some of these molecules are critical in initiation of the immune response (CD28/CD80-86), while CD40-CD40L is required for the amplification of the inflammatory response. By interacting with one or more components of a macrophage, fibroblast, and endothelial cell pathway, a compound of Formula I modulates the activity of this pathway.

In some embodiments, a compound of Formula I modulates the activity of IL-12 signaling and production in macrophages pathway. Interleukin-12 is a heterodimeric cytokine, mainly produced by macrophages. IL-12 plays an important role in the activities of natural killer cells and T lymphocytes. IL-12 mediates enhancement of the cytotoxic activity of NK cells and CD8+ cytotoxic T lymphocytes. IL-2 is also linked to signal transduction in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells. IL-12 also has anti-angiogenic activity, which it controls by increasing production of interferon gamma, which in turn, increases the production of a chemokine called inducible protein-10 (IP-10 or CXCL10) which then mediates the anti-angiogenic effect. Abnormal signaling by IL-12 is known to be involved in diseases which include, but are not limited to, autoimmune diseases, psoriasis and inflammatory bowel disease. By interacting with a pathway involving IL-12, a compound of Formula I modulates the activity of this pathway.

In some embodiments, a compound of Formula I modulates a pathway which regulates the communication between immune cell pathways, for example by modulating the function of cytokines. Cytokines are small cell-signaling protein molecules that are secreted by numerous cells and are a category of signaling molecules used extensively in intercellular communication. Compounds of Formula I can modulate the activity of cytokines by interacting with pathways which control their function.

In some embodiments, the compound of Formula I modulates the activity of a dendritic cell maturation pathway. DCs are capable of evolving from immature, antigen-capturing cells to mature, antigen-presenting, T cell-priming cells, converting antigens into immunogens and expressing molecules such as cytokines, chemokines, costimulatory molecules and proteases to initiate an immune response. The types of T cell-mediated immune responses (tolerance vs. immunity, Th1 vs. Th2) induced can vary, however, depending on the specific DC lineage (myeloid DC1s or lymphoid DC2s) and maturation stage in addition to the activation signals received from the surrounding microenvironment. The ability of DCs to regulate immunity is dependent on DC maturation. A variety of factors can induce maturation following antigen uptake and processing within DCs, including: whole bacteria or bacterial-derived antigens (e.g. lipopolysaccharide, LPS), inflammatory cytokines, ligation of select cell surface receptors (e.g. CD40) and viral products (e.g. double-stranded RNA). During their conversion from immature to mature cells, DCs undergo a number of phenotypical and functional changes. The process of DC maturation, in general, involves a redistribution of major histocompatibility complex (MHC) molecules from intracellular endocytic compartments to the DC surface, down-regulation of antigen internalization, an increase in the surface expression of costimulatory molecules, morphological changes (e.g. formation of dendrites), cytoskeleton re-organization, secretion of chemokines, cytokines and proteases, and surface expression of adhesion molecules and chemokine receptors. By interacting with a dendritic cell maturation pathway, a compound of Formula I modulates the activity of this pathway.

In some embodiments, the compound of Formula I modulates the activity of a pathway which mediates systemic lupus erythematosus (SLE). In SLE, the body's immune system produces antibodies against itself, particularly against proteins in the cell nucleus. SLE is triggered by environmental factors that are unknown. During an immune reaction to a foreign stimulus, such as bacteria, virus, or allergen, immune cells that would normally be deactivated due to their affinity for self tissues can be abnormally activated by signaling sequences of antigen-presenting cells. Thus triggers may include viruses, bacteria, allergens (both IgE and hypersensitivity), and can be aggravated by environmental stimulants such as ultraviolet light and certain drug reactions. These stimuli begin a reaction that leads to destruction of other cells in the body and exposure of their DNA, histones, and other proteins, particularly parts of the cell nucleus. The body's sensitized B-lymphocyte cells will now produce antibodies against these nuclear-related proteins. These antibodies clump into antibody-protein complexes which stick to surfaces and damage blood vessels in critical areas of the body, such as the glomeruli of the kidney, these antibody attacks are the cause of SLE. SLE is a chronic inflammatory disease believed to be a type III hypersensitivity response with potential type II involvement. High mobility group box 1 (HMGB1) is a nuclear protein participating in chromatin architecture and transcriptional regulation which was found in the sera of patients and mice with systemic lupus erythematosus. HMGB1 may contribute to the pathogenesis of chronic inflammatory and autoimmune diseases due to its proinflammatory and immunostimulatory properties. By interacting with a pathway involved in SLE, a compound of Formula I modulates the activity of this pathway.

In some embodiments, the compound of Formula I modulates the activity of a signaling pathway which mediates airway inflammation in asthma. Asthma is a chronic inflammatory lung disease that is characterized by epithelial shedding, airway smooth muscle hypertrophy and hyperplasia, overproduction of mucus, and airway inflammation. The pathophysiology of asthma has been attributed to an inflammatory process that occurs predominantly in the large airways by the accumulation of eosinophils and CD4+ lymphocytes in the submucosa, mucous-gland hyperplasia, thickening of the sub-epithelial collagen layer, submucosal matrix deposition, mast-cell degranulation, and hypertrophy and hyperplasia of the airway smooth muscle. The chronic inflammation in asthma may be owing to an increase in the number of activated T-Cells, which predominantly secrete TH2 (T Helper2) cells. TH2 cells secrete cytokines (Interleukin-4, 5, 6, 9, and 13) that promote allergic inflammation and stimulate B-Cells to produce IgE (Immunoglobulin) and other antibodies. IL-5 stimulates the release of eosinophils into the circulation and prolongs their survival. Interaction of the airway with allergen increases the local concentration of IL-5, which correlates directly with the degree of airway eosinophilia. The eosinophil is a rich source of leukotrienes, particularly the cysteinyl leukotriene C4, which contracts airway smooth muscle, increases vascular permeability, and may recruit more eosinophils to the airway. In contrast, TH1 cells, another class of CD4 T-Cells produce IFN-Gamma (Interferon-Gamma) and IL-2, which initiate the killing of viruses and other intracellular organisms by activating macrophages and cytotoxic T-Cells. These two subgroups of TH cells arise in response to different immunogenic stimuli and cytokines, and they constitute an immunoregulatory loop: cytokines from TH1 cells inhibit TH2 cells, and vice versa. Some cytokines initiate inflammatory responses by activating transcription factors that bind to the promoter region of genes. Transcription factors involved in asthmatic inflammation include NF-KappaB (nuclear factor-kappaB), Activator Protein-1, NFAT (Nuclear Factor of Activated T-cells), CREB (cyclic AMP Response-Element Binding Protein), and various members of the family of STAT (Signal Transduction-Activated Transcription) factors. These transcription factors act on genes that encode inflammatory cytokines, Chemokines, adhesion molecules, and other proteins that induce and perpetuate inflammation. Corticosteroids modulate immunoinflammatory responses in asthma by inhibiting these transcription factors. By interacting with a signaling pathway involved in asthma, a compound of Formula I modulates the activity of this pathway.

In some embodiments, a compound of Formula I selectively decreases activity of one or more genes. Such genes include but are not limited to: BSN (Zinc Finger Protein 231), CD3E (T-Cell Antigen Receptor Complex, Epsilon Subunit Of T3), F10 (Coagulation Factor X), IFNAR1 (Interferon, Alpha, Beta, And Omega, Receptor 1), MMP9 (Matrix Metalloproteinase 9), IL-2 (Interleukin 2), IL-25 (Interleukin 25), IL2RA (Interleukin 2 Receptor, Alpha), TLR2 (Toll-Like Receptor 2), IL-10 (Interleukin 10), and IGF1 (Insulin-Like Growth Factor I).

In some embodiments, a compound of Formula I selectively decreases activity of one or more genes. Such genes include but are not limited to genes known to be associated with IBD, for example CXCL10, MMP9, IGF1, and IL10. In some embodiments a compound of Formula I down-regulates a gene associate with UC, for example CXCL10 or MMP9. In some embodiments a compound of Formula I down-regulates a gene associated with Crohn's disease, for example IGF1 or IL10.

Molecular signatures can identify subjects in need of particular therapies. In one aspect, provided are a method for classifying whether a subject suffering from a disease would be likely to benefit from being treated with a compound of Formula I. For example, the method comprises determining a diagnostic marker profile by detecting the presence or level of at least one diagnostic marker in a blood or tissue sample from a subject affected by a gastrointestional or other disease. Exemplary suitable diagnostic markers include but are not limited to the following genes: CD3E (T-Cell Antigen Receptor Complex, Epsilon Subunit Of T3), PLA2G2A (Phospholipase A2, Group IIa), TLR7 (Toll-Like Receptor 7), IL6 (Interleukin 6), NR3C1 (Nuclear Receptor Subfamily 3, Group C, Member 1), IL2RA (Interleukin 2 Receptor, Alpha), CTLA4 (Cytotoxic T Lymphocyte-Associated 4), IFNAR1 (Interferon, Alpha, Beta, And Omega, Receptor 1), IL1B (Interleukin 1-β), TLR3 (Toll-Like Receptor 3), CD2 (T-Lymphocyte Surface CD2 Antigen), TNF (Tumor Necrosis Factor), and CD80 (CD80 Antigen).

Other embodiments provided for are a method for classifying whether a subject suffering from a disease would be likely to benefit from being treated with a compound of Formula I. For example, the method comprises determining a diagnostic marker profile by detecting the presence or level of at least one diagnostic marker in a blood or tissue sample from a subject affected by a gastrointestional or other disease. Exemplary suitable diagnostic markers include but are not limited to the following genes: CD247, CD3G, NR3C1, CXCR1, CXCR2, PPARG, IFNAR1, CDK3, S1PR4, S1PR5, ITGAL, TLR3, MSLN, CD2, an CD80.

### Methods of Treatment

Provided herein is a method of treating a gastrointestinal disease in a subject in need thereof, comprising administering to the subject a beta blocker. In some embodiments, the beta blocker is selected from the group consisting of nadolol, timolol, harmalol, levobunolol, bisoprolol, alprenolol, carteolol, pindolol, metoprolol, acebutolol, S-propranolol, (−)-atenolol, sotalol, propranolol, R-atenolol, penbutolol, labetalol, pronetalol, oxprenolol, practolol, betaxolol, and dexpropranolol.

In other embodiments, the beta blocker is a compound having the structure:

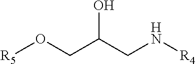

wherein R₄ is H or optionally substituted C₁-C₆ alkyl; R₅ is optionally substituted aryl, substituted heteroaryl, or optionally substituted cycloalkyl; or a pharmaceutically acceptable salt or a pharmaceutically acceptable prodrug thereof. In some embodiments, R₄ is C₁-C₆ alkyl for instance, R₄ can be iso-propyl or tert-butyl. In some embodiments, R₅ is optionally substituted aryl or optionally substituted heteroaryl. In some embodiments, R₅ is optionally substituted phenyl, indolyl, naphthyl, thiadiazyl. In some embodiments, R₅ is aryl optionally substituted with one or more substituents chosen from the group consisting of OH, cyano, C₁-C₆ heterocyclo, R₆, —OR₆, —CONH₂, —NHC(O)R₆, —R₆—OR₆, and —R₆—O—R₆—OR₆; wherein each R₆ is independently C₁-C₆ alkyl or C₁-C₆ cycloalkyl.

Some embodiments of the present invention comprise administering to a subject a compound of Formula I or a pharmaceutically acceptable salt thereof:

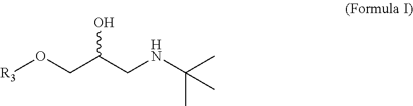

wherein R₃ is

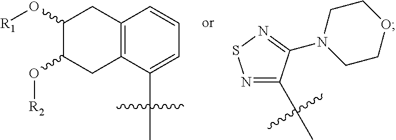

and R₁ and R₂ are independently H or C₁-C₆ alkyl.

In some embodiments, the gastrointestinal disease is inflammatory bowel disease. In some embodiments, the gastrointestinal disease is Crohn's disease. In some embodiments, the gastrointestinal disease is colitis; more particularly ulcerative colitis. In some embodiments, the disease comprises an autoimmune response. In some embodiments, the disease comprises an inflammatory response. In some embodiments, the subject is at risk of developing ulcers or gastrointestinal bleeding. In some embodiments, the gastrointestinal disease is prevented from progressing in said subject.

In some embodiments, the administering step results in an improvement the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject.

In some embodiments, the administering step occurs prior to, concurrent with, or after administration of an additional treatment regimen or therapeutic agent to the subject. In some embodiments, the additional therapeutic agent is a steroid, more particularly a corticosteroid, or other anti-inflammatory drug. In some embodiments, the additional treatment regimen comprises traditional or laparoscopic surgery.

In some embodiments, the administering step occurs prior to, concurrent with, or after administration of an additional treatment to said subject, wherein the additional treatment is a laxative or anti-constipation agent. In some embodiments, the additional therapeutic agent is an antacid or other acidic reducer.

Provided herein is a method of treating a disease in a subject in need thereof, comprising administering to the subject a compound of Formula I, a pharmaceutically acceptable salt thereof or pharmaceutically acceptable prodrug thereof:

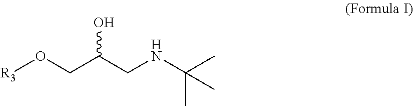

wherein R₃ is

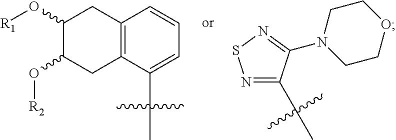

and
R₁ and R₂ are independently H or C₁-C₆ alkyl, and wherein the disease is selected from the group consisting of gastrointestinal disease, inflammatory disease, immunological disease, immune cell trafficking disease, cell-mediated immune response disease, digestive system development and function disease, hypersensitivity response disease, and further wherein the disease is not hypertension, angina, migraine headache, irregular heartbeat, or Parkinson's disease.

Compounds disclosed herein are administered, for example, via pH-dependent release, via microbially-triggered delivery, as a conjugate, via time-controlled delivery, osmotically-regulated delivery, pressure-controlled delivery, a multi matrix system delivery, bioadhesion delivery, or multiparticulate delivery.

In some embodiments, the compound is released preferentially in the small or large intestine, colon, rectum, stomach, anus, or esophagus. In some embodiments, at least 60%, 70%, 80%, or 90% of the administered dose of the compound is released.

In some embodiments, provided herein is a method of treating a disorder in a subject in need thereof using a composition comprising a compound of Formula I. Disorders to be treated include, but are not limited to: gastrointestinal diseases, inflammatory diseases, immunological diseases, immune cell trafficking diseases, cell-mediated immune response diseases, digestive system development and function diseases, and hypersensitivity response diseases.

In some embodiments, provided herein is a method of treating inflammatory diseases using a therapeutically effective amount of a composition comprising a compound of Formula I. Inflammation is a protective biological response of vascular tissues to harmful stimuli, such as pathogens, damaged cells, or irritants. Inflammation is a protective attempt by the organism to remove the injurious stimuli and to initiate healing. However, chronic inflammation can also lead to many diseases, such as hay fever, periodontitis, atherosclerosis, rheumatoid arthritis, and even cancer (e.g., gallbladder carcinoma). Inflammation diseases can be, for example, acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

In some embodiments, provided herein is a method of treating immunological diseases using a therapeutically effective amount of a composition comprising a compound of Formula I. Immunological diseases are caused by dysfunction of the immune system. These diseases can be characterized, for example, by the component(s) of the immune system affected, by whether the immune system is overactive or underactive, or by whether the condition is congenital or acquired. Autoimmune diseases include, but are not limited to, lupus, scleroderma, hemolytic anemia, vasculitis, type I diabetes, Graves disease, rheumatoid arthritis, multiple sclerosis, Goodpasture's syndrome, pernicious anemia, and myopathy. Immunodeficiency diseases include, but are not limited to, severe combined immunodeficiency (SCID), DiGeorge syndrome, hyperimmunoglobinemia E syndrome (also known as Job's Syndrome), common variable immunodeficiency (CVID), chronic granulomatous disease (CGD), classical recurrent infection from catalase positive bacteria and fungi, Wiskott-Aldrich syndrome (WAS), autoimmune lymphoproliferative syndrome (ALPS), hyper-immunoglobulin M syndrome (Hyper-IgM), increased susceptibility to extracellular bacteria and opportunistic infections, leukocyte adhesion deficiency (LAD), NF-κB Essential Modifier (NEMO) mutations, selective immunoglobulin A deficiency (characterized by a deficiency of IgA) resulting in sino-pulmonary and gastrointestinal infections, X-linked agammaglobulinemia (XLA, also known as Bruton type agammaglobulinemia), resulting in a lack of B-cells in circulation, X-linked lymphoproliferative disease (XLP), and ataxia-telangiectasia. Primary immune deficiency diseases are those caused by inherited genetic mutations. Secondary or acquired immune deficiencies are caused by something outside the body such as a virus or immune suppressing drugs. Secondary immune deficiencies include but are not limited to AIDS.

In some embodiments, provided herein is a method of treating immune cell trafficking disease using a therapeutically effective amount of a composition comprising a compound of Formula I. Cell migration is a key aspect of the development of the immune system and mediating an immune response. There is extensive and continual redistribution of cells to different anatomic sites throughout the body. These trafficking patterns control immune function, tissue regeneration, and host responses to insult. Defects in lymphatic function can lead to lymph accumulation in tissues, dampened immune responses, connective tissue and fat accumulation, and tissue swelling known as lymphedema. The lymphatic system contributes to the pathogenesis of various diseases involving immune and inflammatory responses such as chronic inflammation and it also plays a role in cancer in the disseminating tumor cells.

In some embodiments, provided herein is a method of treating antigen presentation diseases using a therapeutically effective amount of a composition comprising a compound of Formula I. Antigen presentation pathway is comprised of at least two distinct pathways: MHC Pathway and the MHC II pathway. Disruption of this signaling pathway can result in diseases such an inflammatory bowel disease and lupus.

In some embodiments, provided herein is a method of treating cell-mediated immune response diseases using a therapeutically effective amount of a composition comprising a compound of Formula I. Cell-mediated immunity is an immune response that involves the activation of macrophages, natural killer cells (NK), antigen-specific cytotoxic T-lymphocytes, and the release of various cytokines in response to an antigen. Cellular immunity protects the body by activating antigen-specific cytotoxic T-lymphocytes that are able to induce apoptosis in body cells displaying epitopes of foreign antigen on their surface, such as virus-infected cells, cells with intracellular bacteria, and cancer cells displaying tumor antigens, activating macrophages and natural killer cells, enabling them to destroy pathogens, and stimulating cells to secrete a variety of cytokines that influence the function of other cells involved in adaptive immune responses and innate immune responses. Cell-mediated immunity is directed primarily at microbes that survive in phagocytes and microbes that infect non-phagocytic cells. It is most effective in removing virus-infected cells, but also participates in defending against fungi, protozoans, cancers, and intracellular bacteria. It also plays a major role in transplant rejection.

In some embodiments, provided herein is a method of treating diseases related to digestive system development and function using a therapeutically effective amount of a composition comprising a compound of Formula I. The entire digestive tract consists of the mouth, pharynx, esophagus, stomach, small intestine, large intestine, rectum and anus. Structural diseases involve an abnormality of the digestive system, while functional diseases interfere with normal function without the presence of any structural abnormality.

In some embodiments, provided herein is a method of treating functional digestive diseases using a therapeutically effective amount of a composition comprising a compound of Formula I. These diseases occur when the organs of the digestive tract appear normal but function abnormally. The most common functional disorder is irritable bowel syndrome. Irritable bowel syndrome causes abdominal pain, gas, bloating, diarrhea and constipation. Irritable bowel syndrome occurs because the nerves and muscles of the digestive tract, usually the large intestine, become sensitive and contract too much causing the symptoms. When the lower esophageal sphincter, a ring of muscle that separates the esophagus from the stomach, begins to work improperly, gastroesophageal reflux disease develops. When this sphincter fails to close tightly or opens spontaneously, stomach acid rises into the esophagus, triggering pain and discomfort in the chest, a condition known as heartburn. Other functional diseases include chronic constipation and dyspepsia, which is the feeling of discomfort in the upper portion of the abdomen.

In some embodiments, provided herein is a method of treating structural digestive diseases using a therapeutically effective amount of a composition comprising a compound of Formula I. Such diseases are characterized by an abnormality in the structure of the digestive tract which interferes with its function. For example, small pouches in the lining of the large intestine, also known as the colon, cause the condition diverticulosis. When these pouches become inflamed or infected, diverticulosis becomes known as diverticulitis. Diverticulitis causes abdominal pain that may be severe and occur suddenly. Some people may also experience fever, chills, nausea and vomiting. Additionally, structural diseases can affect the anal area. Hemorrhoids, swollen blood vessels that line the opening of the anus, occur due to stress and straining. Internal hemorrhoids are normal structures that help protect the rectum from damage. But during straining, these blood vessels descend into the anus, where they become irritated and bleed. Other structural anal diseases include anal fissures, or small cracks in the lining of the anus, and anal fistulas, a hole in the anal canal, which allows waste into the skin.

In some embodiments, provided herein is a method of treating gastrointestinal diseases using a therapeutically effective amount of a composition comprising a compound of Formula I. Gastrointestinal diseases include, but are not limited to inflammatory bowel disease, irritable bowel syndrome, Crohn's disease, digestive disease and UC. In some embodiments, an effective therapeutic amount of a composition comprising a compound of Formula I contacts gastrointestinal tissue or cells and inhibits the level of inflammation, swelling, irritation, open sores, bleeding, ulcers, abdominal pain, or results in an improvement in defecation in the subject. Exemplary target sites of the compound include, but are not limited to, the esophagus, stomach, large and small intestine, sigmoid colon, and rectum in a subject.

In some embodiments, an effective therapeutic amount of a composition comprising a compound of Formula I is applied to a gastrointestinal tissue and reduces the occurrence of a symptom such as inflammation, swelling, irritation, open sores, bleeding, ulcers, fistulas, abdominal pain, or produces an improvement in defecation in the subject. Exemplary target sites of the compound include, but are not limited to, the esophagus, stomach, large and small intestine, sigmoid colon, and rectum in a subject.

In some embodiments, provided herein is a method of treating Crohn's disease using a therapeutically effective amount of a composition comprising a compound of Formula I. Crohn's disease is a type of inflammatory bowel disease which can attack any part of the digestive tract. It typically manifests in the gastrointestinal tract and can be categorized by the specific tract region affected. A disease of the ileum and the large intestine, ileocolic Crohn's accounts for fifty percent of cases. Crohn's ileitis, manifested in the ileum only, accounts for thirty percent of cases, while Crohn's colitis, of the large intestine, accounts for the remaining twenty percent of cases. Gastroduodenal Crohn's disease causes inflammation in the stomach and the duodenum. Jejunoileitis causes spotty patches of inflammation in the jejunum. There are three categories of disease presentation in Crohn's disease: stricturing, penetrating, and inflammatory. Stricturing disease causes narrowing of the bowel that may lead to bowel obstruction or changes in the caliber of the feces. Penetrating disease creates abnormal passageways (fistulae) between the bowel and other structures, such as the skin. Inflammatory disease (or nonstricturing, nonpenetrating disease) causes inflammation without causing strictures or fistulae.

Treatment of Crohn's includes, but is not limited to, uses of antibiotics and aminosalicylate anti-inflammatory drugs and corticosteroids. When symptoms are in remission, treatment enters maintenance, with a goal of avoiding the recurrence of symptoms. Alternatives treatments include aminosalicylates alone and many require immunosuppressive drugs. It has been also suggested that antibiotics change the enteric flora, and their continuous use may pose the risk of overgrowth with pathogens such as Clostridium difficile. Medications used to treat the symptoms of Crohn's disease include 5-aminosalicylic acid formulations, prednisone, immunomodulators such as azathioprine, mercaptopurine, methotrexate, infliximab, adalimumab, certolizumab and natalizumab. Hydrocortisone is also used in severe attacks of Crohn's disease. Surgery is used when partial or a full blockage of the intestine occurs. Surgery may also be required for complications such as obstructions, fistulas and/or abscesses, or if the disease does not respond to drugs. Any of these therapeutic agents and methods can be used in conjunction with compositions comprising a compound of Formula I.

In some embodiments, provided herein is a method of treating UC by using a therapeutically effective amount of a composition comprising a compound of Formula I. This disease is a form of inflammatory bowel disease (IBD) of the colon (large intestine), which includes characteristic ulcers, or open sores. UC can be treated with a number of medications including aminosalicylates such as sulfasalazine, corticosteroids such as prednisone, immunosuppressive medications such as azathioprine, and biological agents such as infliximab. UC can generally be cured by surgical removal of the large intestine, also known as a colectomy. This procedure is necessary in the event of exsanguinating hemorrhage, frank perforation or documented or strongly suspected carcinoma. Any of these therapeutic agents and methods can be used in conjunction with compositions comprising a compound of Formula I.

In some embodiments, provided herein is a method of treating other gastrointestinal diseases using a therapeutically effective amount of a composition comprising a compound of Formula I. Such gastrointestinal diseases include but are not limited to, achalasia, Barrett's oesophagus, colorectal cancer, gastric cancer, oesophageal cancer, celiac disease, colitis, diverticulosis, diverticulitis, gastritis, irritable bowel syndrome, microscopic colitis, collagenous colitis, lymphocytic colitis, pancreatitis and reflux oesophagitis.

In some embodiments, provided herein is a method of treating achalasia using a therapeutically effective amount of a composition comprising a compound of Formula I. Achalasia is a disorder of the esophagus. It is an esophageal motility disorder involving the smooth muscle layer of the esophagus and the lower esophageal sphincter (LES). It is characterized by incomplete LES relaxation, increased LES tone, and lack of peristalsis of the esophagus (inability of smooth muscle to move food down the esophagus) in the absence of other explanations like cancer or fibrosis. The cause of most cases of achalasia is unknown. LES pressure and relaxation are regulated by excitatory (eg, acetylcholine, substance P) and inhibitory (eg, nitric oxide, vasoactive intestinal peptide) neurotransmitters. Persons with achalasia lack nonadrenergic, noncholinergic, inhibitory ganglion cells, causing an imbalance in excitatory and inhibitory neurotransmission. The result is a hypertensive nonrelaxed esophageal sphincter. Achalasia is characterized by difficulty swallowing, regurgitation, and sometimes chest pain. Diagnosis is reached with esophageal manometry and barium swallow radiographic studies. Various treatments are available. Certain medications or botulinum toxin may be used in some cases, but more permanent relief is brought by esophageal dilatation and surgical cleaving of the muscle (Heller myotomy). Any of these therapeutic agents and methods can be used in conjunction with compositions comprising a compound of Formula I.

In some embodiments, provided herein is a method of treating Barrett's oesophagus disease using a therapeutically effective amount of a composition comprising a compound of Formula I. This disease is marked by the presence of columnar epithelia in the lower esophagus, replacing the normal squamous cell epithelium. The main cause of Barrett's esophagus is thought to be an adaptation to chronic acid exposure from reflux esophagitis. Barrett's esophagus is found in a fraction of patients who seek medical care for heartburn (gastroesophageal reflux disease, GERD), although a large subgroup of patients with Barrett's esophagus do not have symptoms. Treatment options for high-grade dysplasia include surgical removal of the esophagus (esophagectomy) or endoscopic treatments such as endoscopic mucosal resection or ablation (destruction). Any of these therapeutic options can be used in conjunction with compositions comprising a compound of Formula I.

In some embodiments, provided herein is a method of treating gastroesophageal reflux disease (GERD) using a therapeutically effective amount of a composition comprising a compound of Formula I. The principal cause of gastroesophageal reflux disease is chronic inflammation. In this disease, acidic stomach, bile, small intestine and pancreatic contents cause damage to the cells of the lower esophagus. Bile acids may induce intestinal differentiation in gastroesophageal junction cells through inhibition of the epidermal growth factor receptor (EGFR) receptor which results in inhibition of Akt, upregulation of the p50 subunit of NF-kB (NFKB1) and ultimately activation of the promotor of the homeobox gene CDX2. Treatment options for GERD include lifestyle modifications (dietary changes or alterations of sleeping position), medications (proton pump inhibitors, including but not limited to omeprazole, esomeprazole, pantoprazole, lansoprazole, and rabeprazole, gastric H2 receptor blockers such as ranitidine, famotidine and cimetidine, antacids, alginic acid, prokinetics such as cisapride, or metoclopriamide, sucralfate, mosapride citrate, GABA_{B} receptor agonists such as baclofen), and surgery (Nissen fundoplication or vagotomy). Any of the named therapeutic agents and methods can be used in conjunction with compositions comprising a compound of Formula I.

In some embodiments, provided herein is a method of treating colorectal cancer (CRC) using a therapeutically effective amount of a composition comprising a compound of Formula I. CRC is a disease originating from the epithelial cells lining the colon or rectum of the gastrointestinal tract, most frequently as a result of mutations in the Wnt signaling pathway that artificially increase signaling activity. The most commonly mutated gene in all colorectal cancer is the APC gene, which produces the APC protein. The APC protein is a “brake” on the accumulation of β-catenin protein, without APC, β-catenin accumulates to high levels and translocates into the nucleus, binds to DNA, and activates the transcription of genes that are normally important for stem cell renewal and differentiation but when inappropriately expressed at high levels can cause cancer. The symptoms and signs of colorectal cancer depend on the location of tumor in the bowel. The classic warning signs of CRC include: worsening constipation, blood in the stool, weight loss, fever, loss of appetite, and nausea or vomiting. Any of the named therapeutic agents and methods can be used in conjunction with compositions comprising a compound of Formula I.

In some embodiments, provided herein is a method of treating gastric cancer using a therapeutically effective amount of a composition comprising a compound of Formula I. Gastric cancer is characterized by a malignancy of rising from any part of the stomach. Stomach cancer is often asymptomatic or causes only nonspecific symptoms in its early stages such as abdominal discomfort, indigestion, and loss of appetite. There are two major pathways for the development of gastric cancer by Helicobacter pylori infection: the indirect action of H. pylori on gastric epithelial cells through inflammation, and the direct action of the bacteria on epithelial cells through the induction of protein modulation and gene mutation. Both pathways work together to promote gastric carcinogenesis. The onset of autoimmune atrophic gastritis, intestinal metaplasia and various genetic factors are associated with increased risk for development of gastric cancer. Treatment for stomach cancer may include surgery, chemotherapy, and/or radiation therapy. Any of the named therapeutic agents and methods can be used in conjunction with compositions comprising a compound of Formula I.

In some embodiments, provided herein is a method of treating oesophageal cancer using a therapeutically effective amount of a composition comprising a compound of Formula I. This form of cancer is characterized by a malignancy arising from the esophagus. The most common types of esophageal cancer are adenocarcinoma, which starts in the glandular cells producing fluids such as mucus, and squamous cell carcinoma, which starts in flat cells of the esophageal lining. Chronic irritation from gastroesophageal reflux disease (GERD), Barrett's esophagus, smoking, obesity and heavy alcohol use are leading risk factors for the disease. Precancerous esophageal lesions and early esophageal cancer may be treated with endoscopic therapies include endoscopic mucosal resection, radiofrequency ablation or photodynamic therapy. For more advanced cancer, esophagectomy surgery to remove a portion of the esophagus is usually necessary. Any of the named therapeutic agents and methods can be used in conjunction with compositions comprising a compound of Formula I.

In some embodiments, provided herein is a method of treating celiac disease (celiac sprue) using a therapeutically effective amount of a composition comprising a compound of Formula I. Celiac disease is an autoimmune disorder of the small intestine. It is caused by a reaction to gliadin, a prolamin (gluten protein) found in wheat, and similar proteins found in the crops of the tribe Triticeae, which includes common grains such as barley and rye. Upon exposure to gliadin, and specifically to three peptides found in prolamins, the enzyme tissue transglutaminase modifies the protein, and the immune system cross-reacts with the small-bowel tissue, causing an inflammatory reaction. That leads to a truncating of the villi lining the small intestine. This interferes with the absorption of nutrients, because the intestinal villi are responsible for absorption. A gluten-free diet is essential to managing celiac disease. Such dietary modifications can be used in conjunction with compositions comprising a compound of Formula I.

In some embodiments, provided herein is a method of treating colitis using a therapeutically effective amount of a composition comprising a compound of Formula I. Colitis is an inflammation of the colon and is often used to describe an inflammation of the large intestine (colon, caecum and rectum). Colitides may be acute and self-limited or chronic, i.e. persistent, and broadly fit into the category of digestive diseases. There are many types of colitis, usually classified by the etiology, for example: autoimmune inflammatory bowel disease are a group of chronic colitides, UC is a chronic colitis that affects the large intestine, Crohn's disease is a type of IBD which often leads to a colitis, idiopathic microscopic colitis is a colitis which is diagnosed by microscopic examination of colonic tissue, lymphocytic colitis, collagenous colitis, iatrogenic diversion colitis, chemical colitis, vascular disease ischemic colitis, infectious colitis (e.g. Clostridium difficile colitis), pseudomembranous colitis, enterohemorrhagic colitis (which may be caused by Shiga toxin in Shigella dysenteriae or Shigatoxigenic group of Escherichia coli, which includes serotype 0157:H7 and other enterohemorrhagic E. coli), colitis caused by parasitic infections, like those caused by Entamoeba histolytica, fulminant colitis (any colitis that becomes worse rapidly). In addition to the diarrhea, fever, and anemia seen in colitis, the patient has severe abdominal pain and presents a clinical picture similar to that of septicemia, where shock is present. About half of human patients require surgery. How a given colitis is treated is dependent on its etiology, e.g. infectious colitis is usually treated with antimicrobial agents, autoimmune mediated colitis is treated with immune modulators/immune suppressants. Severe colitis can be life-threatening and may require surgery. Any such therapeutic agents and methods can be used in conjunction with compositions comprising a compound of Formula I.

In some embodiments, provided herein is a method of treating diverticulosis using a therapeutically effective amount of a composition comprising a compound of Formula I. Diverticulosis is characterized by outpocketings of the colonic mucosa and submucosa through weaknesses of muscle layers in the colon wall. These are more common in the sigmoid colon, which is a common place for increased pressure. The clinical forms include, for example, symptomatic colonic diverticulosis. This condition is characterized by motility (that is, the onward propulsive nature of contractions) of the bowel becomes disorganized. Sometimes, spasm can develop. This results in pain in the left lower abdomen and often is accompanied by the passage of small pellet-like stools and slime, which relieves the pain. Symptoms can consist of bloating, changes in bowel movements (diarrhea or constipation), non-specific chronic discomfort in the lower left abdomen, with occasional acute episodes of sharper pain, and abdominal pain, often in the left lower abdomen and/or after meals. Another clinical manifestation is known as complicated colonic diverticulosis. This condition is characterized by the diverticula bleeding, either rapidly (causing bleeding through the rectum) or slowly (causing anaemia). The diverticula can become infected and develop abscesses, or even perforate. The exact etiology of colonic diverticulosis has yet to be fully clarified. Increasing the amount of fiber in the diet may reduce symptoms of diverticulosis and prevent complications such as diverticulitis. Fiber keeps stool soft and lowers pressure inside the colon so that bowel contents can move through easily. Therapeutic agents such as methylcellulose (Citrucel) or psyllium (Metamucil), taken one to three times a day, are also known to help. Any such therapeutic agents and methods can be used in conjunction with compositions comprising a compound of Formula I.

In some embodiments, provided herein is a method of treating diverticulitis with a therapeutically effective amount of a composition comprising a compound of Formula I. Diverticulitis is caused by inflammation of diverticula. Onset of this disease can occur when diverticula become infected or/and by development of abscesses. For mild diverticulitis, treatments include liquid or low-fiber diet, resting the colon, and avoiding foods or beverages that aggravate your symptoms and antibiotics. More severe, recurring acute attacks or complications, such as peritonitis, abscess, or fistula may require surgery to remove the ruptured section is removed and a colostomy is performed. Treatment with IV antibiotics may also be necessary. Any such therapeutic agents and methods can be used in conjunction with compositions comprising a compound of Formula I.

In some embodiments, provided herein is a method of treating gastritis with a therapeutic effective amount of a composition comprising a compound of Formula I. Gastritis is an inflammation of the lining of the stomach. Treatment includes over-the-counter antacids which are commonly used for mild gastritis. When antacids do not provide sufficient relief, medications such as cimetidine, ranitidine, nizatidine or famotidine are often prescribed. Proton pump inhibitors such as omeprazole, lansoprazole, rabeprazole, and esomeprazole may be also used to reduce acid. Cytoprotective agents such as sucralfate, bismuth subsalicylate and misoprostol are designed to help protect the tissues that line the stomach and small intestine. Colonization of the gastric mucosa with H. pylori results in the development of chronic gastritis in infected individuals and in a subset of patients chronic gastritis progresses to complications (i.e. ulcer disease, gastric neoplasias, some distinct extra gastric diseases). Several regimens are used to treat H. pylori infection. Most use a combination of two antibiotics and a proton pump inhibitor, and bismuth may also added to the regimen. The antibiotic aids in destroying the bacteria, and the acid blocker or proton pump inhibitor relieves pain and nausea, heals inflammation, and may increase the antibiotic's effectiveness. Any such therapeutic agents and methods can be used in conjunction with compositions comprising a compound of Formula I.

In some embodiments, provided herein is a method of treating irritable bowel syndrome (IBS) using a therapeutically effective amount of a composition comprising a compound of Formula I. IBS is characterized by chronic abdominal pain, discomfort, bloating, and alteration of bowel habits. It can be classified as either diarrhea-predominant, constipation-predominant or IBS with alternating stool pattern. As a functional bowel disorder, IBS has no known cause. Treatments can include different medications depending on the symptoms. Medications may consist of stool softeners and laxatives in constipation-predominant IBS, and antidiarrheals (e.g., opiate, opioid, or opioid analogs such as loperamide, codeine, diphenoxylate) in diarrhea-predominant IBS for mild symptoms and stronger opiates such as morphine and oxycodone for severe cases. Drugs affecting serotonin (5-HT) in the intestines can help reduce symptoms. Serotonin stimulates the gut motility and so agonists can help constipation-predominate irritable bowel, while antagonists can help diarrhea-predominant irritable bowel. Osmotic laxatives such as polyethylene glycol, sorbitol, and lactulose can help avoid “cathartic colon” which has been associated with stimulant laxatives. Lubiprostone (Amitiza), is a gastrointestinal agent used for the treatment of idiopathic chronic constipation and constipation-predominant IBS. Lubipro stone is a bicyclic fatty acid (prostaglandin E1 derivative) that acts by specifically activating ClC-2 chloride channels on the apical aspect of gastrointestinal epithelial cells, producing a chloride-rich fluid secretion. These secretions soften the stool, increase motility, and promote spontaneous bowel movements (SBM). The use of antispasmodic drugs (e.g., anticholinergics such as hyoscyamine or dicyclomine) may help patients, especially those with cramps or diarrhea. Any such therapeutic agents and methods can be used in conjunction with compositions comprising a compound of Formula I.

In some embodiments, provided herein is a method of treating collagenous or lymphocytic colitis using a therapeutically effective amount of a composition comprising a compound of Formula I. Collagenous colitis is an inflammatory condition of the colon that cause persistent watery diarrhea. In lymphocytic colitis, the colonoscopy is normal but the mucosal biopsy reveals an accumulation of lymphocytes in the colonic epithelium and connective tissue (lamina propria). Both collagenous colitis and lymphocytic colitis are sometimes referred to collectively as microscopic colitis. The colonoscopy is normal but the mucosal biopsy reveals an accumulation of lymphocytes in the colonic epithelium and connective tissue and shows a distinctive thickening of the subepithelial collagen table. Treatment often begins with lifestyle changes. Budesonide (Entocort) is effective in controlling diarrhea in more than 75% of the patients with collagenous colitis. Any such therapeutic agents and methods can be used in conjunction with compositions comprising a compound of Formula I.

In some embodiments, provided herein is a method of treating pancreatitis using a therapeutically effective amount of a composition comprising a compound of Formula I. Pancreatitis is characterized by inflammation of the pancreas. It occurs when pancreatic enzymes that digest food are activated in the pancreas instead of the small intestine. It may be acute—beginning suddenly and lasting a few days, or chronic—occurring over many years. It has multiple causes and symptoms. The treatment of pancreatitis is supportive. Pain control is usually required. Morphine generally is suitable for pain control, among other medications. Oral intake, especially fats, is generally restricted at first. Fluids and electrolytes are replaced intravenously. However there is also evidence showing that earlier nutrition and feeding contributes to better recovery. The underlying cause should also be treated (targeting gallstones, discontinuing medications, cessation of alcohol etc.) The patient is monitored for complications. Endoscopic therapy by introducing stents to drain blocked pancreatic ducts Shock wave lithotripsy to pulverize pancreatic stones Surgery (laparoscopic and traditional), Islet cell transplantation may be offered if most or all of the pancreas is removed, Enzyme therapy for malabsorption helps restore normal digestion and reduces the amount of fat in the feces, leading to weight gain and improved well-being. Dietary changes such as eating smaller meals and limiting fats help reduce the need for digestive enzymes. Any such therapeutic agents and methods can be used in conjunction with compositions comprising a compound of Formula I.

In some embodiments, provided herein is a method of treating reflux oesophagitis using a therapeutically effective amount of a composition comprising a compound of Formula I. Reflux oesophagitis is inflammation of the esophagus which may be acute or chronic. The acute esophagitis can be catarrhal or phlegmonous, whereas the chronic esophagitis may be hypertrophic or atrophic. Causes of esophagitis include stomach acids backing up into the esophagus, infection, oral medications and allergies. Therapeutic agents include, but are not limited to, proton pump inhibitors which block acid production in the stomach and allow time for damaged esophageal tissue to heal. Drugs available by prescription include omeprazole (Prilosec), esomeprazole (Nexium) and lansoprazole (Prevacid). Over-the-counter proton pump inhibitors also are available. Other treatments for gastroesophageal reflux disease (GERD) may alleviate GERD symptoms temporarily, but generally have little effect on esophagitis. Fundoplication, a surgical procedure, may be used to treat GERD and improve the condition of the esophagus if other interventions don't work. During this procedure, a portion of the stomach is wrapped around the valve separating the esophagus and stomach (lower esophageal sphincter). This strengthens the sphincter and prevents acid from backing up into the esophagus. Fundoplication may also correct problems related to a hiatal hernia. Treatment for reflux esophagitis may also include avoiding alcohol, caffeine, aspirin, nonsteroidal anti-inflammatory medications, and foods that irritate your stomach. Any such therapeutic agents and methods can be used in conjunction with compositions comprising a compound of Formula I.

In some embodiments, provided herein is a method of treating a hypersensitivity response disease using a therapeutically effective amount of a composition comprising a compound of Formula I. Hypersensitivity response diseases are characterized by undesirable reactions produced by the normal immune system, including allergies and autoimmunity. Hypersensitivity requires a pre-sensitized (immune) state of the host and these reactions may be damaging, uncomfortable, or occasionally fatal. Hypersensitivity response diseases is classified into five-group classification system. Type I hypersensitivity (allergy) includes atopy, anaphylaxis and asthma. Type II hypersensitivity (cytotoxic, antibody-dependent) include autoimmune hemolytic anemia, thrombocytopenia, erythroblastosis fetalis, and Goodpasture's syndrome. Type III hypersensitivity (immune complex disease) includes diseases such as serum sickness, arthus reaction, systemic lupus erythematosus, and extrinsic allergic alveolitis (hypersensitivity pneumonitis). Type IV hypersensitivity (delayed-type hypersensitivity (DTH), cell-mediated immune memory response, antibody-independent) includes diseases such as contact dermatitis, Mantoux test, Chronic transplant rejection, and multiple sclerosis. Type V hypersensitivity (receptor mediated autoimmune disease) includes Graves' disease, and Myasthenia Gravis. Any therapeutic agents and methods known to treat the disorders noted above can be used in conjunction with compositions comprising a compound of Formula I.

In some embodiments, the diseases to be treated using a therapeutically effective amount of a composition comprising a compound of Formula I include, but are not limited to, Acute Lung Injury, Alpha thalassemia, Asthma, Bipolar Disorder, Cardiomyopathy, Secondary, CFS (chronic fatigue syndrome), Cystic Fibrosis, Dehydration, Depression, Down Syndrome, Gamma-hydroxybutyric acidaemia, HIV encephalitis, Huntington's Disease, Hyperreactive airway disease, Hypertrophy, Irritable bowel syndrome, Idiosyncratic drug effect, Irritable bowel syndrome variant of childhood with constipation, Irritable bowel syndrome variant of childhood with diarrhea, Muscular Dystrophy, Ochratoxicosis, Osteolysis, Pigmented villonod synovitis, Polycystic Ovary Syndrome, PTSD—Post-traumatic stress disorder, Retinitis Pigmentosa, Retinoschisis, Rhinovirus infection, Schizophrenia, Sickle Cell Anemia, Smoke inhalation, Turner Syndrome, Type 1 diabetes mellitus, Type 2 diabetes mellitus, UC, and Vitamin A Deficiency.

In some embodiments, the diseases to be treated using a therapeutically effective amount of a composition comprising a compound of Formula I include, but are not limited to, gastrointestinal disease, inflammatory disease, organismal injury and abnormalities, connective tissue development and function, immunological disease, antigen presentation, humoral immune response, immune cell trafficking, inflammatory response, infectious disease, digestive system development and function, and hypersensitivity response.

In some embodiments, treatment of a condition described herein is effected by administration of a compound of the invention in conjunction with another therapeutic agent, adjuvant or therapeutic regimen. Regardless of the disease, disorder or condition being treated, the overall benefit experienced by the patient is additive of the two therapeutic agents or the patient experiences a synergistic benefit. Any agents that selectively regulate the expression or activity of the signaling pathways listed above can be used in the methods and compositions disclosed herein.

Provided herein are combination treatments using compositions comprising a compound of Formula I along with agents for the treatment of acidity related diseases such as antacids, which include, but are not limited to, magnesium compounds, aluminum compounds, calcium compounds, sodium compounds and combinations thereof. Acid reduction medications which are magnesium compounds include, but are not limited to, magnesium carbonate, magnesium oxide, magnesium peroxide, magnesium hydroxide, and magnesium silicate. Aluminum compounds include, but are not limited to, aluminum hydroxide, algeldrate, aluminum phosphate, dihydroxyaluminum sodium carbonate, aluminum acetoacetate, aloglutamol and aluminum glycinate. Calcium compounds include, but are not limited to, calcium carbonate and calcium silicate. Sodium compounds include, but are not limited to, sodium bicarbonate. Combinations and complexes of aluminum, calcium and magnesium compounds include ordinary salt combinations, magaldrate, almagate, hydrotalcite, and almasilate.

Other embodiments provided for are combination treatments using compositions comprising a compound of Formula I along with agents for treatment of peptic ulcer and gastro-oesophageal reflux disease which include but are not limited to: H2-receptor antagonists, prostaglandins, proton pump inhibitors, and combinations of agents for eradication of Helicobacter pylori, and other agents. H2-receptor antagonists include but are not limited to: cimetidine, ranitidine, famotidine, nizatidine, niperotidine, roxatidine, and lafutidine. Prostaglandins include agents such as misoprostol and enprostil. Proton pump inhibitors include but are not limited to omeprazole, pantoprazole, lansoprazole, rabeprazole, esomeprazole and dexlansoprazole. Combinations of agents for eradication of Helicobacter pylori include but are not limited to amoxicillin, clarithromycin and a proton pump inhibitor such as omeprazole, lansoprazole, pantoprazole or esomeprazole. Other agents for treatments of peptic ulcer and gastro-oesophageal reflux disease include but are not limited to: carbenoxolone, sucralfate, pirenzepine, methiosulfonium chloride, bismuth subcitrate, proglumide, sulglicotide, acetoxolone, zolimidine, troxipide, bismuth subnitrate, alginic acid, carbenoxolone, and combinations thereof, including combinations with psycholeptics and/or gefarnate.

Other embodiments provided for are combination therapies using compositions comprising a compound of Formula I with agents for the treatment of functional bowel diseases such as anticholinergics (e.g. with tertiary or quaternatry amino groups), antispasmodics, papaverine, drugs acting on serotonin receptors, Belladonna alkaloids, propulsive agents and derivatives and/or combinations thereof.

Anticholinergics include but are not limited to oxyphencyclimine, camylofin, mebeverine, trimebutine, rociverine, dicycloverine, dihexyverine, difemerine and piperidolate. Anticholinergics also include but are not limited to benzilone, glycopyrronium, oxyphenonium, penthienate, propantheline, otilonium bromide, methantheline, tridihexethyl, isopropamide, hexocyclium, poldine, mepenzolate, bevonium, pipenzolate, diphemanil, 2-benzhydryloxyethyl)diethyl-methylammonium iodide, tiemonium iodide, prifinium bromide, timepidium bromide, fenpiverinium, oxyphenonium, combinations and benzetimide carbachol and neostigmin. Synthetic antispasmodics include but are not limited to dimethylaminopropionylphenothiazine, nicofetamide and tiropramide. Papaverine and derivatives include but are not limited to papaverine, drotaverine and moxaverine. Drugs acting on serotonin receptors include but are not limited to alosetron, tegaserod, cilansetron, and prucalopride. Other agents used in the treatment of functional bowel diseases include but are not limited to fenpiprane, diisopromine, chlorbenzoxamine, pinaverium, fenoverine, idanpramine, proxazole, alverine, trepibutone, isometheptene, caroverine, phloroglucinol, silicones, trimethyldiphenylpropylamine, alverine, silicones, combinations, physiostigmin and macrogol ricinoleat. Belladonna alkaloids and derivatives thereof include but are not limited to, atropine, hyoscyamine, belladonna total alkaloids, butylscopolamine, methylatropine, methylscopolamine, fentonium, and cimetropium bromide. Propulsive (prokinetic) agents include but are not limited to metoclopramide, cisapride, domperidone, bromopride, alizapride, clebopride and physiostigmine.

Other embodiments provided for are combination therapies using compositions comprising a compound of Formula I with agents such as laxatives, including agents such as softeners, emollients, contact laxatives, bulk producers, osmotically acting laxatives, enemas, peripheral opioid receptor antagonists and other laxatives. Softeners and emollients include but are not limited to, liquid paraffin, docusate sodium and combinations thereof. Contact laxatives include but are not limited to oxyphenisatine, bisacodyl, dantron, phenolphthalein, castor oil, Senna glycosides, cascara, sodium picosulfate, bisoxatin, and combinations thereof. Bulk producers include but are not limited to ispaghula (psylla seeds), ethulose, sterculia, linseed, methylcellulose, triticum (wheat fibre), and polycarbophil calcium. Osmotically acting laxatives include but are not limited to, magnesium carbonate, magnesium oxide, magnesium peroxide, magnesium sulfate, mineral salts, lactulose, lactitol, sodium sulfate, pentaerithrityl, macrogol, mannitol, sodium phosphate, sorbitol, magnesium citrate, sodium tartrate, lactulose and combinations thereof. Enemas include but are not limited to sodium phosphate, bisacodyl, dantron, glycerol, oil, sorbitol, docusate sodium, laurilsulfate and combinations thereof. Peripheral opioid receptor antagonists include but are not limited to methylnaltrexone bromide and alvimopan. Other laxatives include but are not limited to glycerol, carbon dioxide producing drugs and lubiprostone.

Other embodiments provided for are combination therapies using compositions comprising a compound of Formula I with agents such as intestinal anti-infective agents. Intestinal anti-infectives include, but are not limited to, antibiotics and sulfonamides. Intestinal adsorbents include agents such as charcoal preparations, bismuth preparations, and other intestinal adsorbents. Intestinal anti-infectives also include but are not limited to antibiotics such as neomycin, nystatin, natamycin, streptomycin, polymyxin b, paromomycin, amphotericin b, kanamycin, vancomycin, colistin, rifaximin, neomycin, combinations, streptomycin, combinations, dihydrostreptomycin, gentamicin, apramycin, and bacitracin. Sulfonamides inuclde but are not limited to phthalylsulfathiazole, sulfaguanidine, succinylsulfathiazole, sulfonamides, formosulfathiazole, phthalylsulfathiazole and combinations thereof.

Other embodiments provided for are combination therapies using compositions comprising a compound of Formula I with visilizumab, blinatumaomab, steroids, repertaxin, SB-265610, pioglitazone/metformin, rosiglitazone, GI262570, pioglitazone, tesaglitazar, troglitazone, interferon alfacon-1, interferon alfa-2a, interferon beta-1a, IFNA2, interferon alfa-2b/ribavirin, pegintron, interferon beta-1b, flavopiridol, fingolimod, efalizumab, ampligen, amatuximab, alfecept, siplizumab, abatacept, or belatacept.

In some embodiments, surgical treatment is administered to a subject prior to, concurrent with or after administration of a composition comprising a compound of Formula I. Surgical treatments include without limitation to proctosigmoidectomy, right colectomy, ileocolectomy, total abdominal colectomy, abdominoperineal resection, rectopexy, total proctocolectomy, endoscopic mucosal resection, vagotomy, pyloroplasty, and antrectomy.

In some embodiments, a compound of Formula I is used in animal models of gastrointestinal disease to inhibit and reduced the level or occurrence of inflammation, swelling, irritation, open sores, bleeding, ulcers, abdominal pain, immune response, and improvement with defecation in the subject. In some embodiments, the compound is used in animal models to investigate and determine the effectiveness of delivery systems or combination treatments with the compound.

In some embodiments, a compound of Formula I is used in a cell culture system to run an in vitro assays used to examine the effect of the compound on downstream gene targets or biomarkers that are known to have important functions in anti-inflammatory, immunosuppressive and immunomodulatory effects in the cell.

In some embodiments, provided herein is a method of treating a subject in need thereof with a therapeutically effective composition comprising a compound of Formula I to reduce or prevent fatigue, weight loss, loss of appetite, rectal bleeding, and loss of body fluids and electrolytes.

In some embodiments, the disorders to be treated using a composition comprising a compound of Formula I exclude diseases which are hypertension, angina, migraine headache, irregular heartbeat, or Parkinson's disease. In some embodiments, disorders to be treated using a composition comprising a compound of Formula I exclude cardiovascular or neurodegenerative diseases.

In some embodiments, a method of treating a gastrointestinal disease in a subject in need thereof comprises administering to the subject a pharmaceutical composition comprising a beta blocker wherein the pharmaceutical composition is delivered by rectal administration.

In some embodiments, the pharmaceutical composition is chosen from the group consisting of enemas, rectal gels, rectal foams, rectal aerosols, suppositories, jelly suppositories, or retention enemas. In one embodiment, the composition is a suppository. In some embodiments, the composition is a rectal gel.

In some embodiments, a method of treating a gastrointestinal disease in a subject in need thereof comprises administering to the subject a pharmaceutical composition comprising a beta blocker wherein the pharmaceutical composition is delivered by rectal administration, and wherein the dose of beta blocker is low.

In some embodiments, the low dose can be the absolute amount of beta blocker the pharmaceutical composition comprises. For example, the pharmaceutical composition can comprise less than 70, 60, 50, 40, 30, 20, 15, 10, 8, 6, 4, 3, 2, 1, 0.5, 0.4, 0.1 mg of beta blocker. In some embodiments, the pharmaceutical composition comprises less than 40, 20, 10, 8, 6, 4, or 2 mg of beta blocker. In some embodiments, the pharmaceutical composition comprises less than 40, 20, 10, 8, 6, 4, or 2 mg of beta blocker and greater than 0.05, 0.1, 0.2, 0.3, 0.4, 0.5, 0.8, 1.0, 1.3, 1.6, or 1.9 mg of beta blocker. In some embodiments, the pharmaceutical composition comprises between about 0.1 mg and about 20 mg of beta blocker.

In some embodiments, the low dose can be the amount of beta blocker the pharmaceutical composition comprises relative to the subject's body weight. In other embodiments, the low dose is determined by the absolute amount of beta blocker that the subject is administered. For example, the subject can be administered less than 40, 20, 10, 8, 6, 4, or 2 mg of beta blocker daily. The subject can be administered less than 40 mg of beta blocker daily. The subject can be administered less than 20 mg of beta blocker daily. The subject can be administered less than 10 mg of beta blocker daily.

In other embodiments, the low dose is determined by the amount of beta blocker that the subject is administered relative to the subject's body weight. For example, the subject can be administered less than 0.6, 0.4, 0.2, 0.1, 0.05, 0.02, or 0.01 mg of beta blocker daily per kg of the subject's body weight. In some embodiments, the subject is administered less than 0.6 mg of beta blocker per kg of the subject's body weight daily. In other embodiments, the subject is administered less than 0.2 mg of beta blocker per kg of the subject's body weight daily. In other embodiments, the subject is administered less than 0.1 mg of beta blocker per kg of the subject's body weight daily. In some embodiments, the subject is administered between about 0.05 mg and about 1 mg of beta blocker per kg of the subject's body weight daily.

In other embodiments, the amount of beta blocker can be correlated with the pharacokinetics of the beta blocker. For example, the amount of beta blocker in the pharmaceutical composition can be selected such that administration of the pharmaceutical composition results in a peak plasma level in the subject of less than 50, 40, 30, 20, or 10 ng/mL. In some embodiments, the amount of beta blocker in the pharmaceutical composition can be selected such that administration of the pharmaceutical composition results in a peak plasma level in the subject of greater than 1, 2, 4, 6, 8, 10, 14, 18, 20, or 25 ng/mL. In some embodiments, the amount of beta blocker in the pharmaceutical composition can be selected such that administration of the pharmaceutical composition results in a peak plasma level in the subject of between about 1 ng/mL and about 50 ng/mL.

The some embodiments, wherein the pharmaceutical composition comprises an amount of beta blocker, which, when administered to the subject orally, is insufficient to result in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject. In some embodiments, the pharmaceutical composition comprises an amount of beta blocker which is sufficient to result in an improvement in the intestinal barrier in the large or small intestine of said subject, and wherein said improvement comprises a reduction in the level of or number of occurrences of inflammation, swelling, irritation, open sores, bleeding, ulcers, abdominal pain and defecation irregularities in the subject.

The severity of IBD can be determined by any clinically validated method. For instance, the severity of IBD can be measured by analysis of the bowel. In some embodiments, the severity of IBD is assessed using the following scoring system: maximum score=10
1) Adhesions:
   none=0
   minimal=1
   involving several bowel loops=2
2) Strictures:
   none=0
   mild=1
   moderate=2
   severe, proximal dilatation=3
3) Ulcers:
   none=0
   inear ulceration <1 cm=1
   two linear ulcers <1 cm=2
   more sites of ulceration or one large ulcer=3
4) Wall thickness:
   less than 1 mm=0
   1-3 mm=1
   >3 mm=2

The severity of IBD can be determined by histopatholgy of the bowel. For instance, examinign the cells of the bowel for inflammation or necrosis can be a method for determining the severity of IBD. In some embodiments, the severity of IBD can be determined by scoring inflammation and necrosis of the colon by a scoring method. One such scoring method is:

Inflammation—neutrophil and macrophage infiltration predominantly, fibroplasia and neovascularization in areas of transmural necrosis
0=No inflammation
0.5=Very minimal focal infiltrates in mucosa only, affects <2% of the total colon
1.0=Minimal multifocal infiltrates in mucosa only, affects 2-10% of the total colon
2.0=Mild multifocal infiltrates affecting mucosa, submucosa, outer muscle layers and serosa, affects 11-25% of the total colon
3.0=Moderate multifocal infiltrates affecting mucosa, submucosa, outer muscle layers and serosa, affects 26-50% of the total colon
4.0=Marked multifocal to diffuse infiltrates affecting mucosa, submucosa, outer muscle layers and serosa, affects 51-75% of the total colon
5.0=Severe multifocal to diffuse infiltrates affecting mucosa, submucosa, outer muscle layers and serosa, affects >75% of the total colon.
Necrosis—measure the total length of all colon sections, measure the total length of necrotic mucosa devoid of epithelium to determine % area affected
0=No necrosis
0.5=Very minimal, focal, affects <2% of the total colon
1.0=Minimal focal or multifocal, affects 2-10% of the total colon
2.0=Mild, focal or multifocal, affects 11-25% of the total colon
3.0=Moderate focal or multifocal, affects 26-50% of the total colon
4.0=Marked focal or multifocal, affects 51-75% of the total colon
5.0=Severe, affects >75% of the total colon

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### 1: Pharmaceutical Compositions

### Example 1a

### Parenteral Composition

To prepare a parenteral pharmaceutical composition suitable for administration by injection, 80 mg of a compound of Formula I, described herein is dissolved in DMSO and then mixed with 10 mL of 0.8% sterile saline. The mixture is incorporated into a dosage unit form suitable for administration by injection.

### Example 1b

### Oral Composition

To prepare a pharmaceutical composition for oral delivery, 80 mg of a compound of Formula I, described herein is mixed with 8000 mg of starch. The mixture is incorporated into an oral dosage unit for, such as a hard gelatin capsule, which is suitable for oral administration.

### Example 1c

### Sublingual (Hard Lozenge) Composition

To prepare a pharmaceutical composition for buccal delivery, such as a hard lozenge, mix 80 mg of a compound of Formula I, described herein, with 500 mg of powdered sugar mixed, with 1.6 mL of light corn syrup, 2.4 mL distilled water, and 0.42 mL cherry extract. The mixture is gently blended and poured into a mold to form a lozenge suitable for buccal administration.

### Example 1d

### Inhalation Composition

To prepare a pharmaceutical composition for inhalation delivery, 25 mg of a compound of Formula I, described herein is mixed with 50 mg of anhydrous citric acid and 150 mL of 0.9% sodium chloride solution. The mixture is incorporated into an inhalation delivery unit, such as a nebulizer, which is suitable for inhalation administration.

### Example 1e

### Rectal Gel Composition

To prepare a pharmaceutical composition for rectal delivery, 1500 mg of a compound of Formula I, described herein is mixed with 2.0 g of PVP 90, 100 mg of methylparapen, 5 g of glycerin and 100 mL of purified water. The resulting gel mixture is then incorporated into rectal delivery units, such as syringes, which are suitable for rectal administration.

### Example 1f

### Topical Gel Composition

To prepare a pharmaceutical topical gel composition, 80 mg of a compound of a compound of Formula I, described herein is mixed with 2.0 g of PVP 90, 10 mL of propylene glycol, 10 mL of isopropyl myristate and 100 mL of purified alcohol USP. The resulting gel mixture is then incorporated into containers, such as squeeze bottles, which are suitable for topical administration.

### Example 1g

### Ophthalmic Solution Composition

To prepare a pharmaceutical ophthalmic solution composition, 80 mg of a compound of Formula I, described herein is mixed with 0.9 g of NaCl in 100 mL of purified water and filtered using a 0.1 micron filter. The resulting isotonic solution is then incorporated into ophthalmic delivery units, such as eye drop containers, which are suitable for ophthalmic administration.

### Example 2

### Computational In Silico Determination of Compound Activity

The method for determining the viability of drug candidates for treating ulcerative colitis is Chemical Perturbation Signature (CPS) in which expression data is mapped to gene ID's so disease RNA expression comparisons may be scored regardless of the technology used to measure the expression.

The CPS score is computed based on a drug signature and two gene sets from a disease comparison:
SIG_UP is the list of the top N genes significantly increased in the disease comparison
SIG_DOWN is the list of the top N genes significantly decreased in the disease comparison
   These two gene sets are referred to as gene tags.

The drug signature is the set of genes measured in an experiment comparing gene expression pro-files for treated vs. untreated, ranked by a metric of differential expression. For each set S of the two gene tag sets, enrichment scores, KS_UP and KS_DOWN, are calculated using an algorithm that corresponds to a weighted Kolmogorov-Smirnov-like statistic. The CPS score can then be computed by comparing the enrichment statistics for SIG_UP and SIG_DOWN. If the enrichment score KS_UP is opposite in sign to KS_DOWN, then the CPS score is KS_UP-KS_DOWN. Otherwise, the CPS score is set to zero, meaning that no conclusion about enrichment is drawn.

Non-zero CPS scores are obtained in two cases:
SIG_UP is enriched at the top of the drug signature and SIG_DOWN is enriched at the bottom of the signature.
   This is interpreted to mean that the drug effect is similar to the disease effect. The CPS score is then positive.
SIG_DOWN is enriched at the top and SIG_UP is enriched at the bottom
   This is interpreted to mean that the drug effect is opposed to the disease effect.

The CPS score is then negative.

In order to better evaluate the CPS scores for a drug predicted to be efficacious for a disease a consistency index (CI), which is a construct that attempts to account for CPS scores (human and animal where they exist), and negative (more effective) as well as positive (less effective) CPS scores. The consistency metric for a given drug prediction for a disease is calculated from the total # of CPS scores (number of drug experiments×number of disease experiments), the number of those CPS scores that are negative (more effective), the number of CPS scores that are positive (less effective), and the number of CPS scores that are zero (null). A larger, positive consistency index is better, while a negative consistency index is a poorer result.

Table 1 below shows the CI and average CPS for two corticosteroids known to be useful for treating ulcerative colitis and twenty-two beta-blockers.

| Drug name | CI | Avg CPS |
|---|---|---|
| nadolol | 1.16 | −0.36 |
| timolol | 1.16 | −0.30 |
| harmalol | 0.66 | −0.27 |
| prednisone | 0.52 | −0.25 |
| levobunolol | 0.5 | −0.37 |
| bisoprolol | 0.34 | −0.24 |
| alprenolol | 0.32 | −0.31 |
| prednisolone | 0.28 | −0.26 |
| carteolol | 0.16 | −0.21 |
| pindolol | 0.12 | −0.19 |
| metoprolol | 0 | −0.33 |
| acebutolol | 0 | −0.28 |
| S-propranolol | 0 | −0.21 |
| (−)-atenolol | 0 | −0.23 |
| sotalol | −0.16 | −0.20 |
| propranolol | −0.16 | Non-determined |
| R-atenolol | −0.16 | −0.18 |
| penbutolol | −0.22 | −0.47 |
| labetalol | −0.5 | −0.21 |
| pronetalol | −0.5 | −0.19 |
| oxprenolol | −0.66 | Non-determined |
| practolol | −0.68 | −0.11 |
| betaxolol | −0.84 | Non-determined |
| dexpropranolol | −1.12 | Non-determined |

The results in the Table 1 show that nadolol and timolol are the best two beta-blockers for being active for treating ulcerative colitis, and that their activity should be better than the two tested corticosteroids, prednisone and prednisolone.

### Example 3

Prophylactic efficacy study in TNBS (2,4,6-trinitrobenzenesulfonic acid) rat colitis model.

Preparation of doses: For Groups 1, 2: 50 ml 0.1M acetic acid and 30 ml sterile water were combined. The pH was adjusted from 4.45 to 6.7 with 0.1N NaOH. The stir bar was removed and the solution made up to 100 ml with sterile water. For Group 3, 133.9 mg prednisolone 21-hemisuccinate sodium salt was dissolved in 100 ml de-ionized water to prepare a 1 mg/ml solution. For Group 4, 600 mg Nadolol was dissolved in 50 ml 0.1M acetic acid and 30 ml sterile water. The pH was adjusted from 4.45 to 6.7 with 0.1N NaOH, and the solution made up to 100 ml with sterile water. For Group 5, 1.2 g Nadolol was dissolved in 50 ml 0.1M acetic acid and 30 ml sterile water. The pH was adjusted from 4.45 to 6.7 with 0.1N NaOH and the solution made up to 100 ml with sterile water. For Group 6: 1 g cellulose gum was dissolved in 50 ml 0.1N acetic acid and 30 ml de-ionized water. The volume was made up to 100 ml with sterile water. For Group 7, 600 mg Nadolol was dissolved in 50 ml 0.1M acetic acid and 30 ml sterile water. 1 g cellulose gum was added and dissolved into the solution and the solution made up to 100 ml with sterile water. For Group 8, 1.2 g Nadolol was dissolved in 50 ml 0.1M acetic acid and 30 ml sterile water. 1 g cellulose gum was added and dissolved into the solution and the solution made up to 100 ml with sterile water.

TABLE 2
Treatment Groups

| Group | Treatment | Dose (mg/kg) | ROA |
|---|---|---|---|
| 1 | Vehicle | 10 ml/kg | PO |
| 2 | Vehicle | 10 | PO |
| 3 | Prednisolone | 10 | PO |
| 4 | Nadolol | 60 | PO |
| 5 | Nadolol | 120 | PO |
| 6 | Vehicle | 10 ml/kg | IR |
| 7 | Nadolol | 60 | IR |
| 8 | Nadolol | 120 | IR |

(Day −1) 84 Sprague-Dawley rats (male, 160-180 g each) were sorted into treatment groups based upon average body weight. The rats were weighed and food-deprived. The rats in groups 1-5 were dosed orally (PO) at 10 ml/kg with their respective solutions according to Table 2. The rats in Groups 6-8 were anesthetized intra-rectally (IR) instilled with 10 ml/kg of their respective treatment according to Table 2, the anus was pinched closed and the rat held inverted until recovered from anesthesia (at least 1 min).

After 24 hours (day 0), an aqueous solution of TNBS (2,4,6-trinitrobenzenesulfonic acid) 16 mg/ml in 50% ethanol was made and the rats were weighed and dosed as in TABLE 2. One hour later, the rats anesthetized and Groups 2-8 were intra-rectally instilled with 4 ml/kg TNBS solution (64 mg/kg), the anus was pinched closed and the rat held inverted until recovered from anesthesia (at least 1 min). Group 1 received de-ionized water. The rats were re-fed after recovery from TNBS instillation.

The rats were weighed and dosed as in TABLE 2 each day for the next six days (day 2-7), The rats were anesthetized and exsanguinated into pre-chilled serum separator tubes, the blood processed to serum which was stored in several aliquots at −80° C. A midline incision was made in the abdomen and the colon was evaluated for adhesions and stricture. The colon was removed and the length recorded. A midline incision was made the entire length of the colon, the contents removed and the colon weight and colon wall thickness recorded. A section of the colon was preserved in 20 volumes of 10% neutral buffered formalin. The end of the colon section at the rectal end of the section was edged with Evans Blue dye for orientation purposes. Another section of the colon was weighed and homogenized on an ice batch in 5 ml PBS. The homogenate was centrifuged and aliquots stored in labeled Eppendorf tubes at −80° C. The following parameters were used to determine colonic score:
Colonic Score Parameters
a) Adhesions:
   1) none=0
   2) minimal=1
   3) involving several bowel loops=2
b) Strictures:
   1) none=0
   2) mild=1
   3) moderate=2
   4) severe, proximal dilatation=3
c) Ulcers:
   1) none=0
   2) linear ulceration <1 cm=1
   3) two linear ulcers <1 cm=2
   4) more sites of ulceration or one large ulcer=3
d) Wall thickness:
   1) less than 1 mm=0
   2) 1-3 mm=1
   3) >3 mm=2

The formalin-preserved colon samples were submitted for histological preparation and evaluation. Sections of H&E stained colon section were analyzed using criteria below and scored for inflammation and necrosis.

Inflammation—neutrophil and macrophage infiltration predominantly, fibroplasia and neovascularization in areas of transmural necrosis.

0=No inflammation
0.5=Very minimal focal infiltrates in mucosa only, affects <2% of the total colon
1.0=Minimal multifocal infiltrates in mucosa only, affects 2-10% of the total colon
2.0=Mild multifocal infiltrates affecting mucosa, submucosa, outer muscle layers and serosa, affects 11-25% of the total colon
3.0=Moderate multifocal infiltrates affecting mucosa, submucosa, outer muscle layers and serosa, affects 26-50% of the total colon
4.0=Marked multifocal to diffuse infiltrates affecting mucosa, submucosa, outer muscle layers and serosa, affects 51-75% of the total colon
5.0=Severe multifocal to diffuse infiltrates affecting mucosa, submucosa, outer muscle layers and serosa, affects >75% of the total colon.

Necrosis—measure the total length of all colon sections, measure the total length of necrotic mucosa devoid of epithelium to determine % area affected

0=No necrosis
0.5=Very minimal, focal, affects <2% of the total colon
1.0=Minimal focal or multifocal, affects 2-10% of the total colon
2.0=Mild, focal or multifocal, affects 11-25% of the total colon
3.0=Moderate focal or multifocal, affects 26-50% of the total colon
4.0=Marked focal or multifocal, affects 51-75% of the total colon
5.0=Severe affects >75% of the total colon

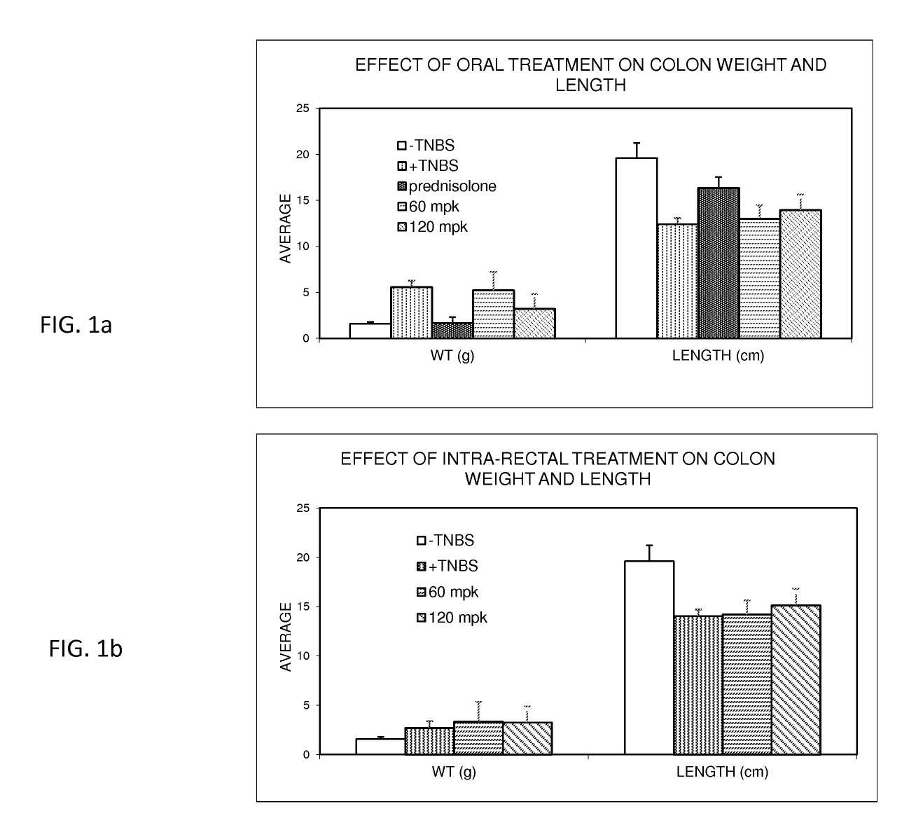

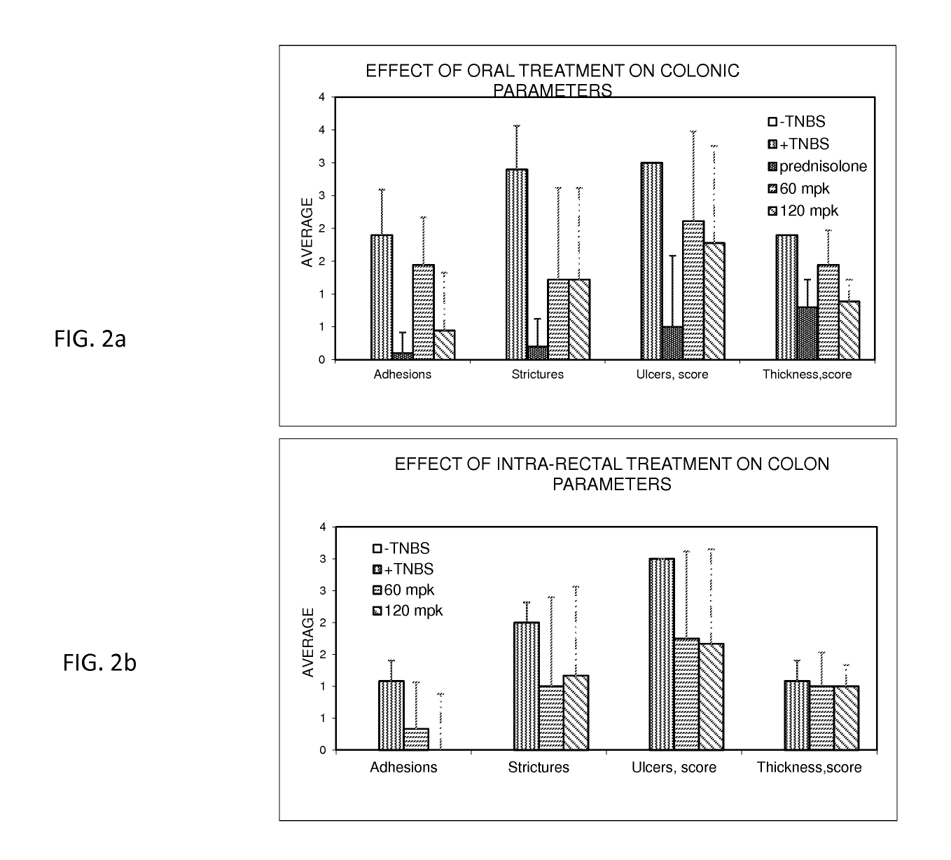

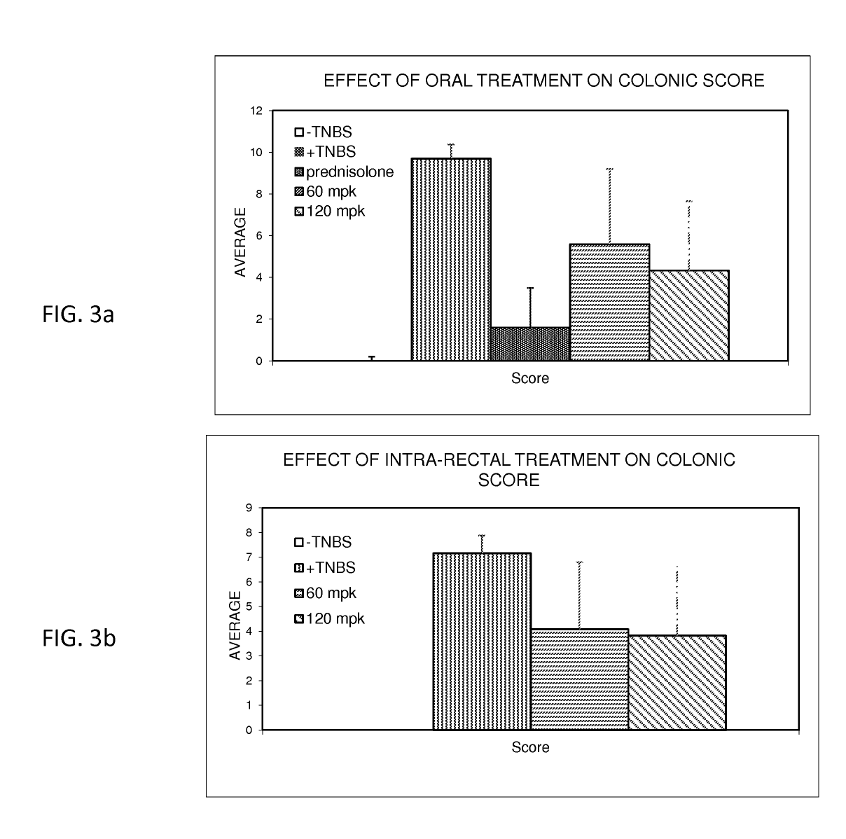

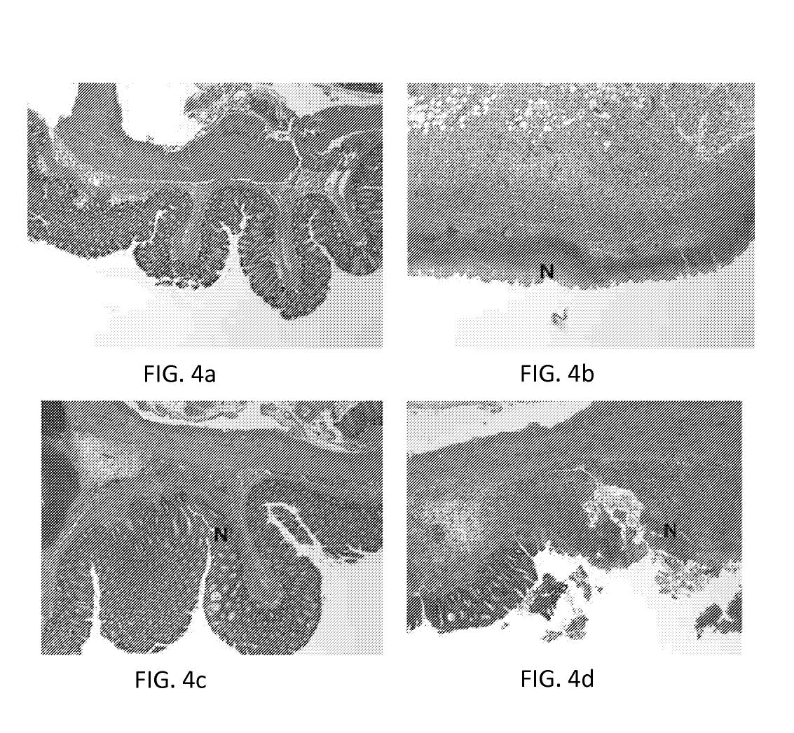

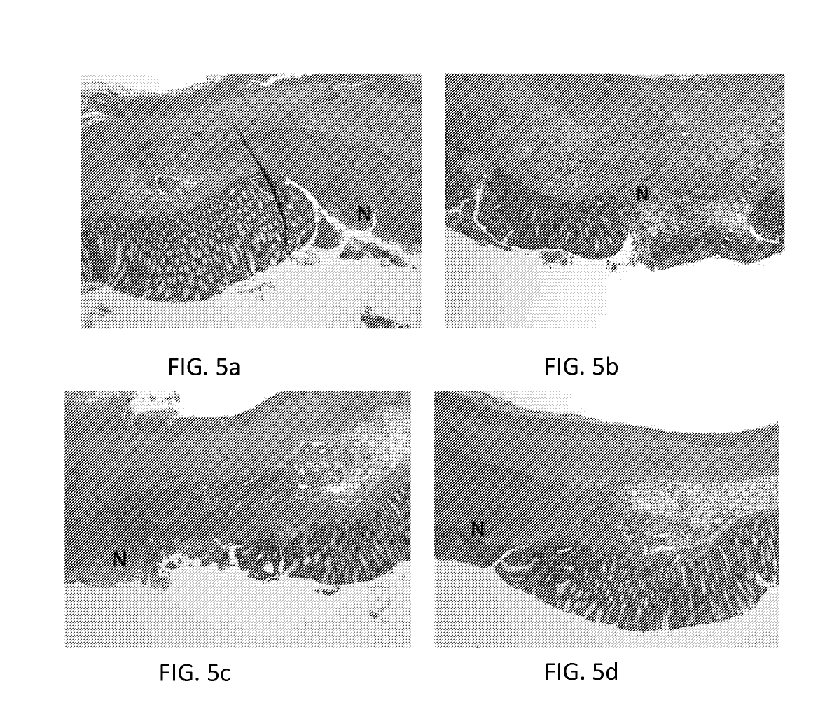

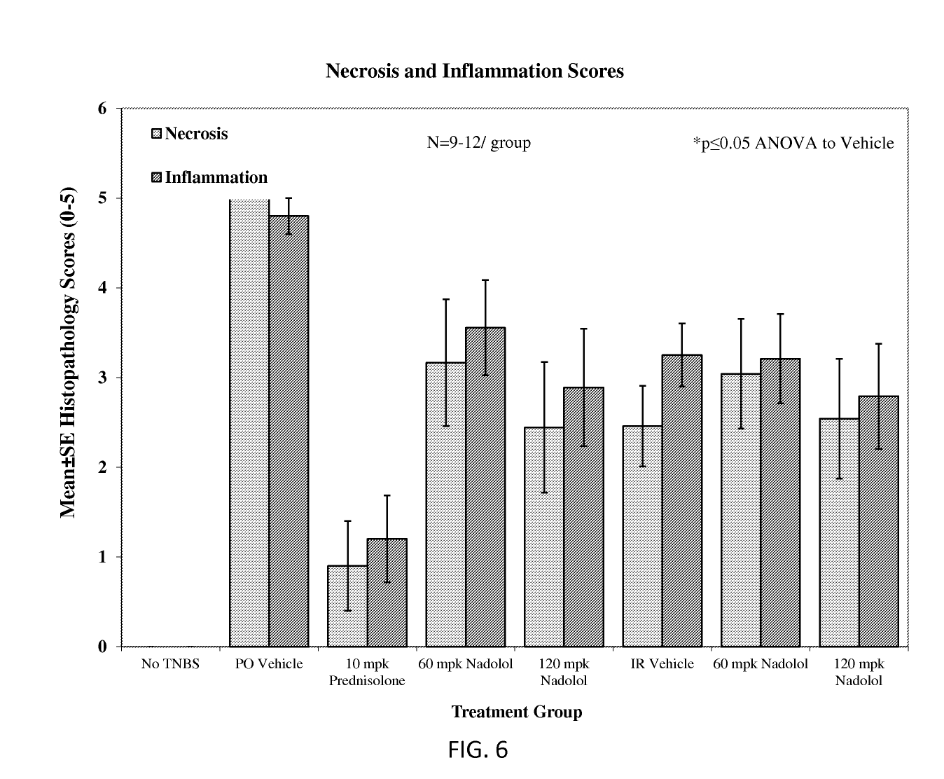

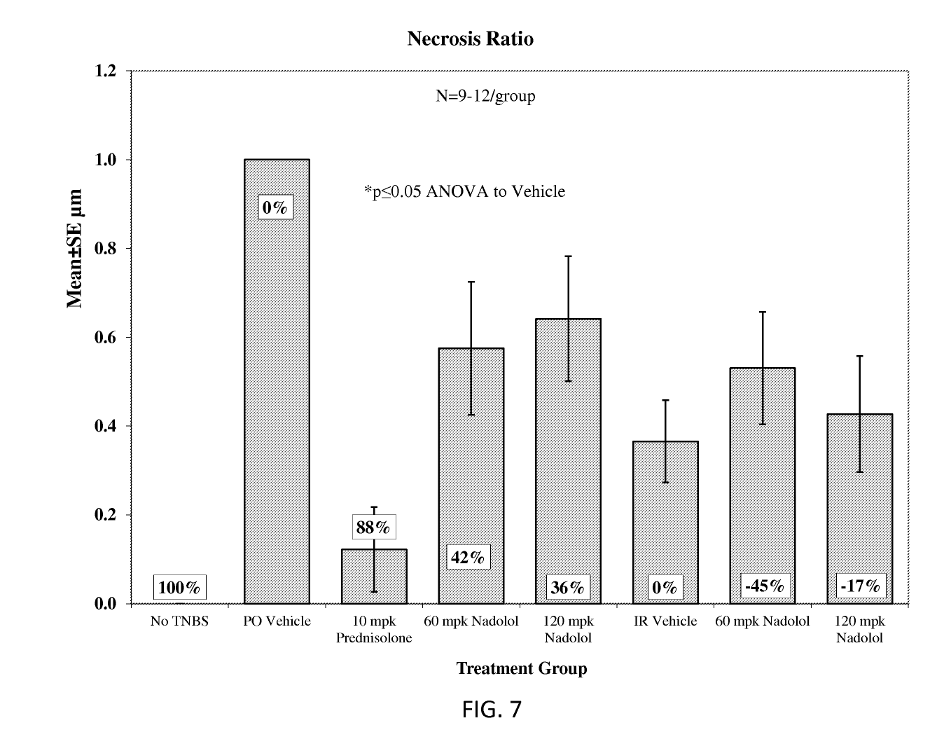

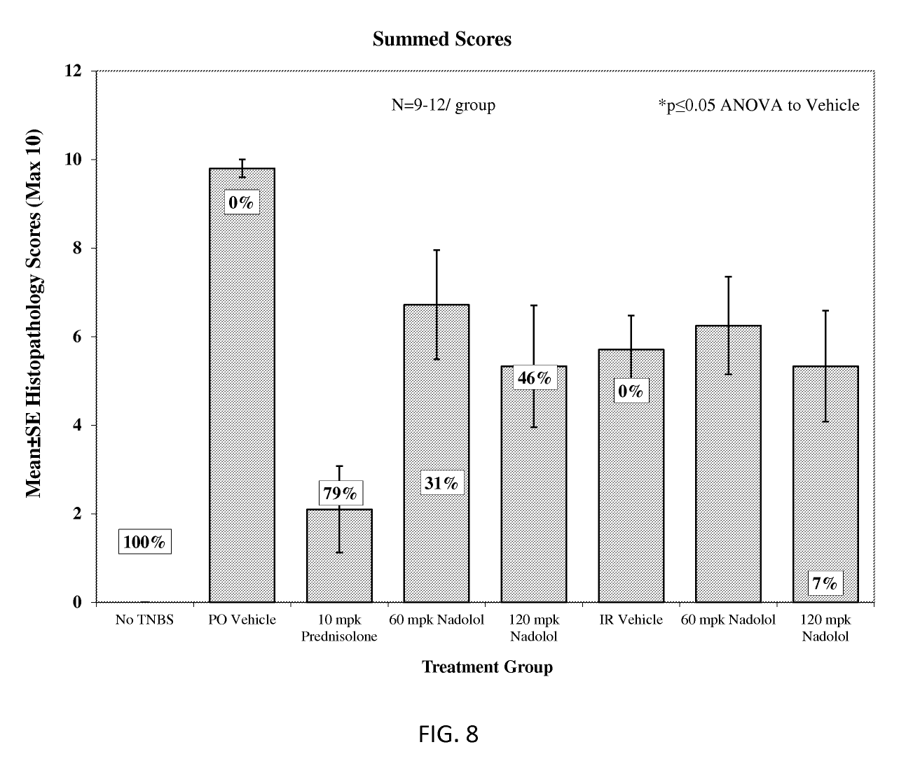

The results of the study are summarized graphically in FIGS. 1-8.

### Example 4

### TNBS Dose Escalation Study

Sprague-Dawley (SD) rats 118 (male, 180-200 g) were assigned to 11 groups of 10 or 12 rats per group so that each group has approximately the same mean weight.

(Day −1) Initiate daily dosing of Groups 1-11 as in TABLE 1. Rats are deprived of food for 24 hours prior to disease initiation. 1) Intra-rectal (IR) administration is accomplished by briefly anesthetizing the rats, administering the test material at 10 ml/kg via a 20 gauge feeding needle inserted via the rectum, maintaining the rat in an inverted position with anus pinched shut until recovered from anesthesia. 2) Oral formulations are first dissolved in 0.05N acetic acid and pH adjusted to 6-7 with 0.1N NaOH.

TABLE 3
Treatment groups

| Group | # rats | Treatment | Dose (mg/kg) | ROA |
|---|---|---|---|---|
| 1 | 10 | Vehicle | 10 ml/kg | PO |
| 2 | 10 | Vehicle | 10 ml/kg | PO |
| 3 | 10 | Prednisolone | 1 | PO |
| 4 | 10 | Nadolol | 6 | PO |
| 5 | 10 | Nadolol | 19 | PO |
| 6 | 10 | Nadolol | 60 | PO |
| 7 | 10 | Nadolol | 120 | PO |
| 8 | 12 | Vehicle | 10 ml/kg | IR |
| 9 | 12 | Nadolol | 6 | IR |
| 10 | 12 | Nadolol | 19 | IR |
| 11 | 12 | Nadolol | 60 | IR |

*PO = oral gavage
**IR = intra-rectal

(DAY 0) Prepare TNBS Solution (50% TNBS: 50% 200 proof ethanol; 16 mg/ml TNBS).

Dose as in TABLE 3. After 1 hour, rats in Groups 2-11 are administered TNBS. Anesthetize rats and administer 64 mg/kg (4 ml/kg) TNBS Solution into colon of each rat, then re-feed rats.

(DAY 7): Rats are weighed and dosed as in Table 3. Rats are weighed and dosed as in TABLE 3. Terminal blood samples are collected from anesthetized rats. Samples are collected in pre-chilled-tubes and processed to serum. Store serum at −80° C. in several aliquots. A midline incision is made on each animal. Colon is removed and length measured and recorded. A longitudinal incision is made on each colon. Fecal material is gently washed away with saline. Excess fluid is gently blotted and colon weighed. The severity of IBD is assessed using the following scoring system: maximum score=10
1) Adhesions:
   none=0
   minimal=1
   involving several bowel loops=2
2) Strictures:
   none=0
   mild=1
   moderate=2
   severe, proximal dilatation=3
3) Ulcers:
   none=0
   inear ulceration <1 cm=1
   two linear ulcers <1 cm=2
   more sites of ulceration or one large ulcer=3
4) Wall thickness:
   less than 1 mm=0
   1-3 mm=1
   >3 mm=2

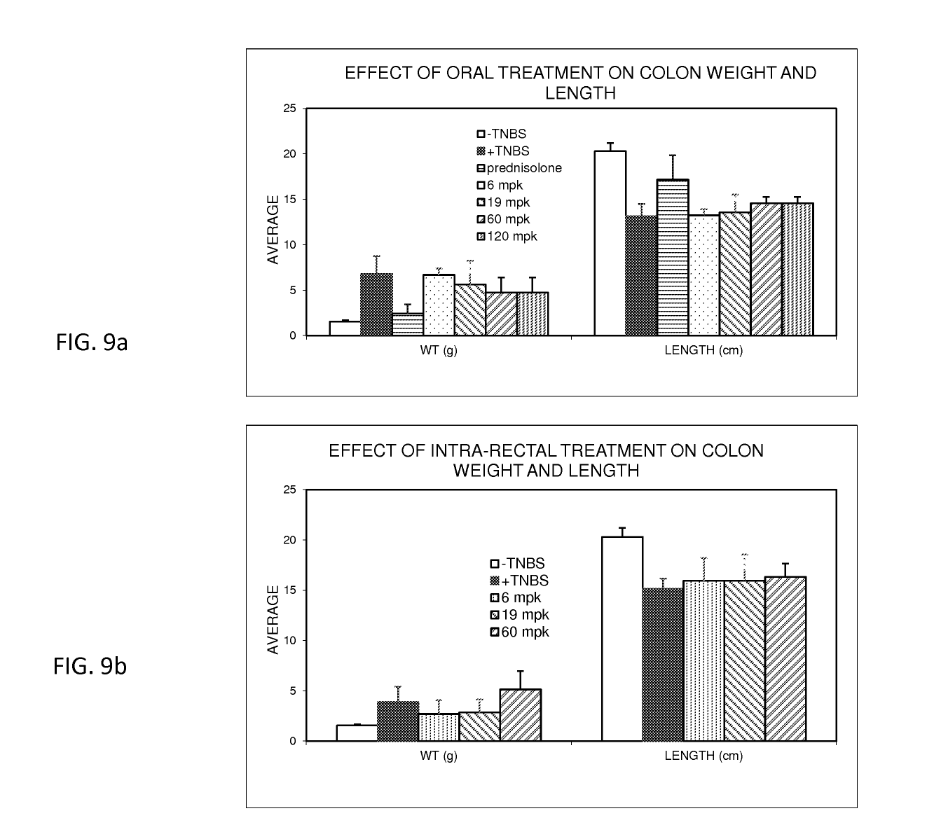

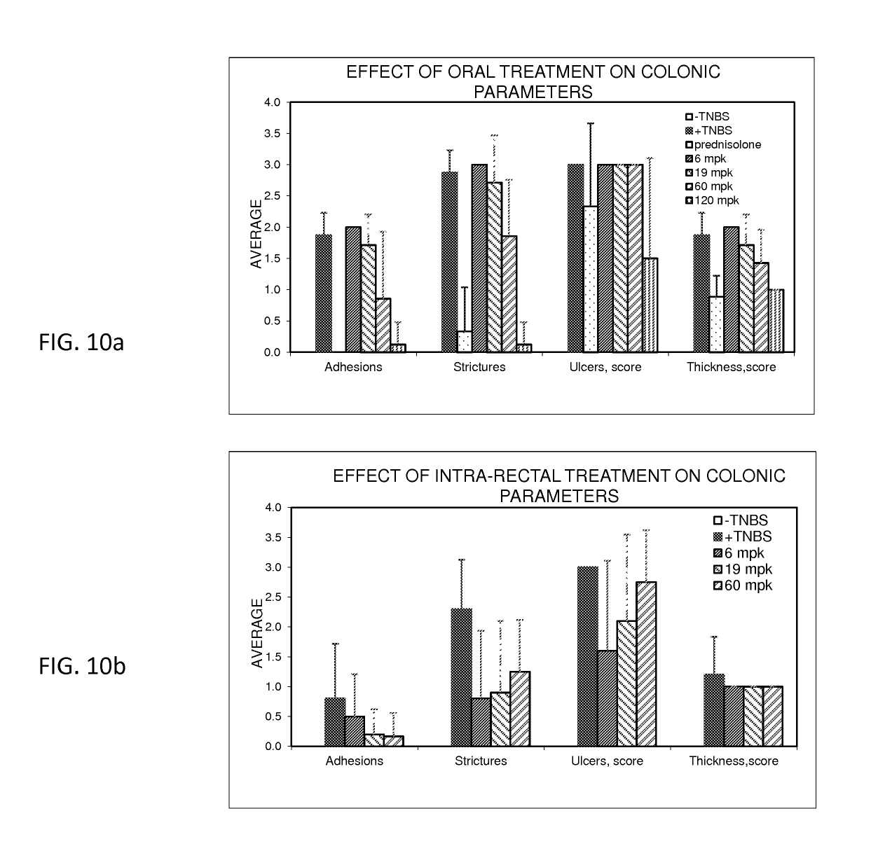

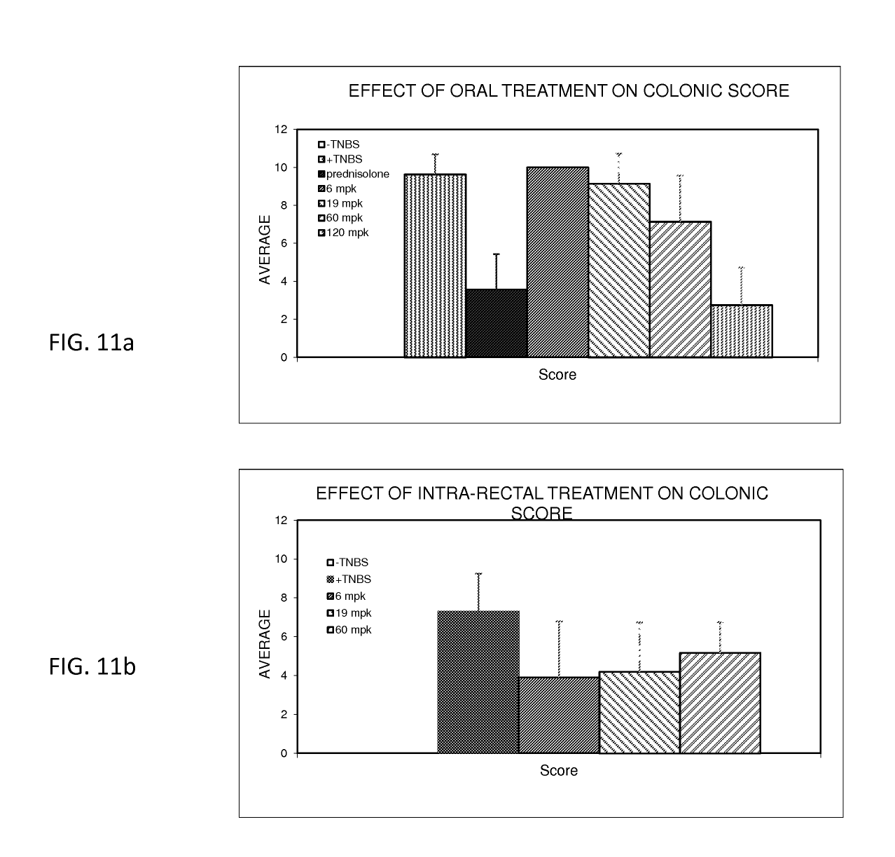

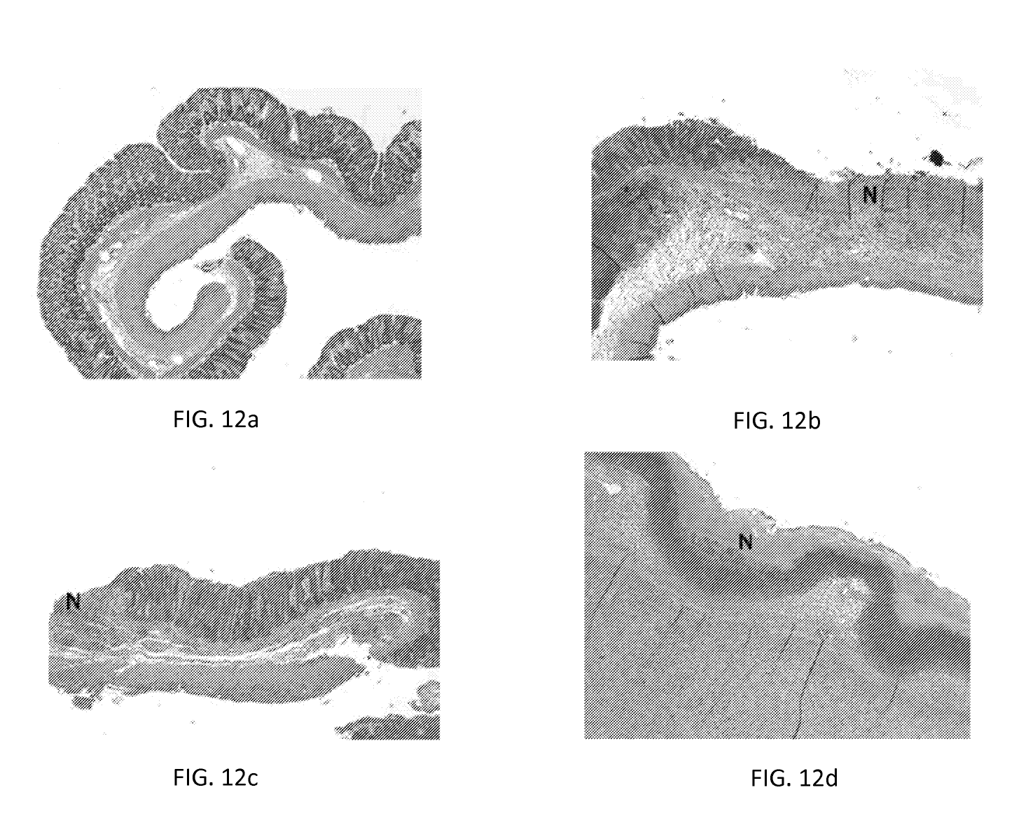

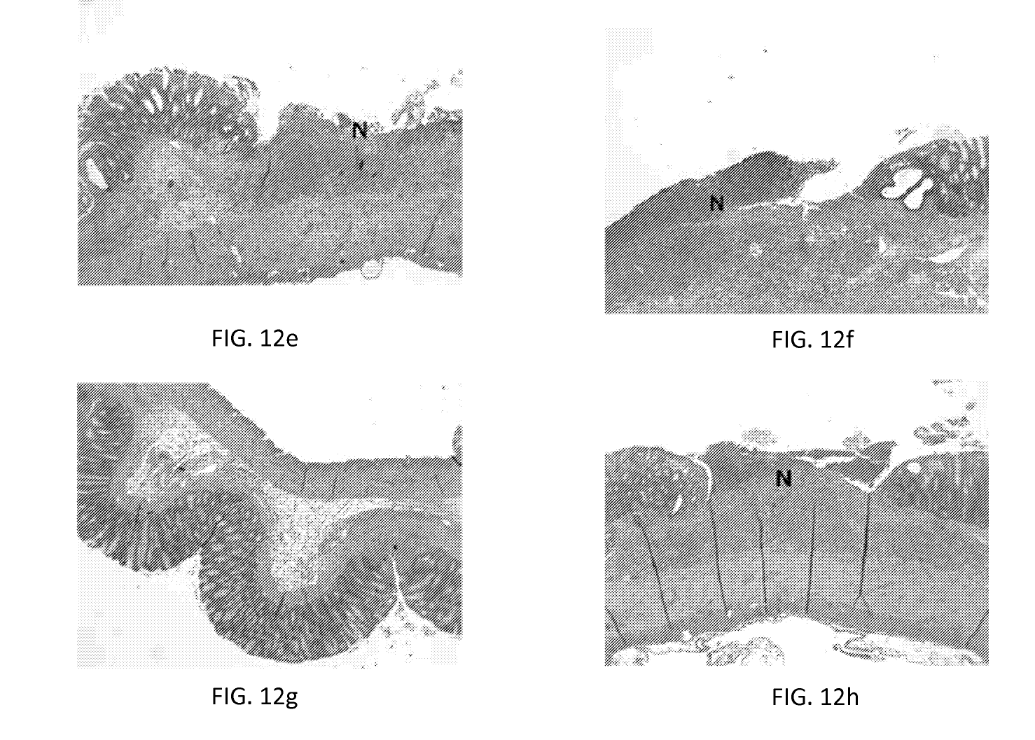

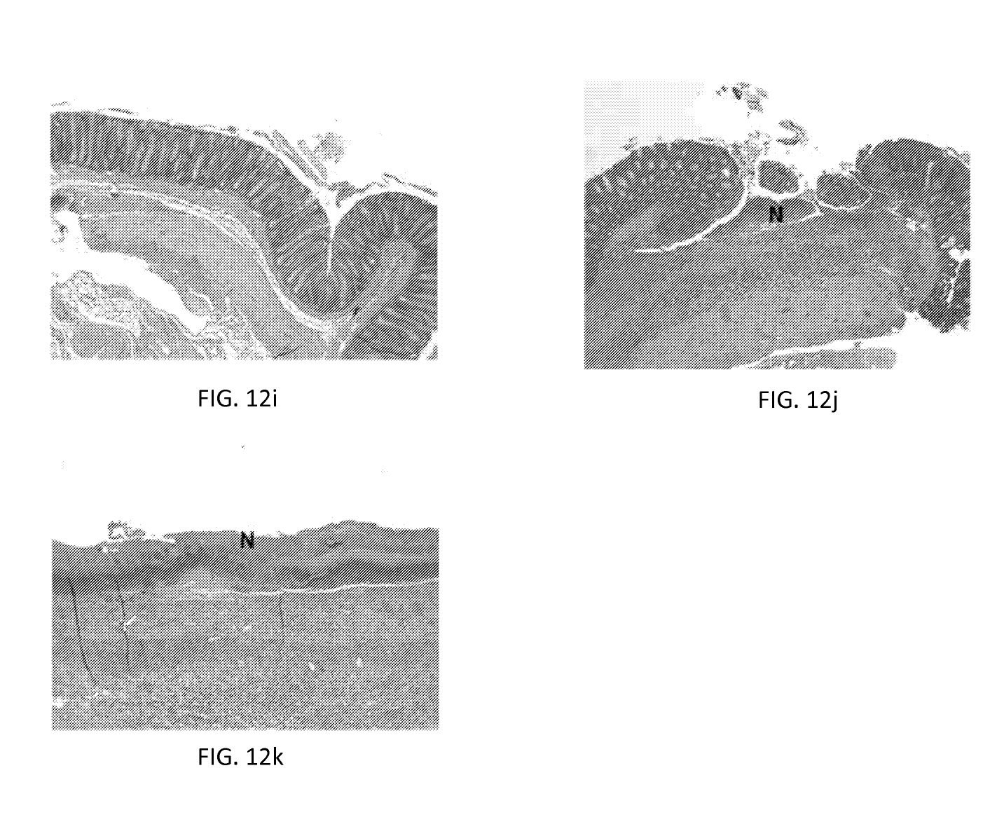

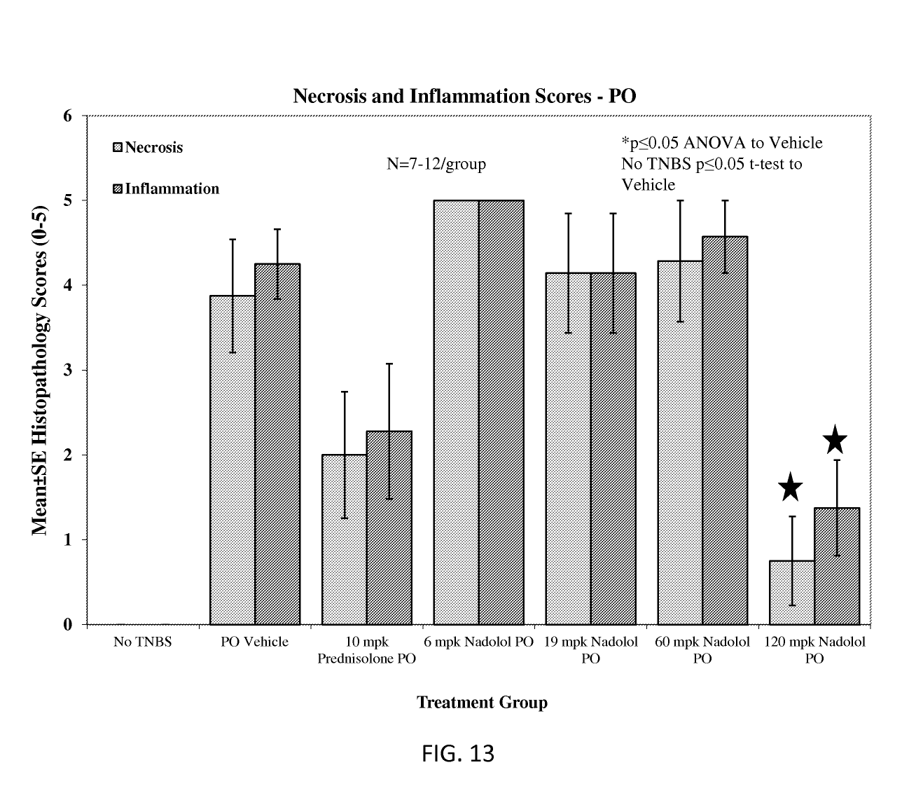

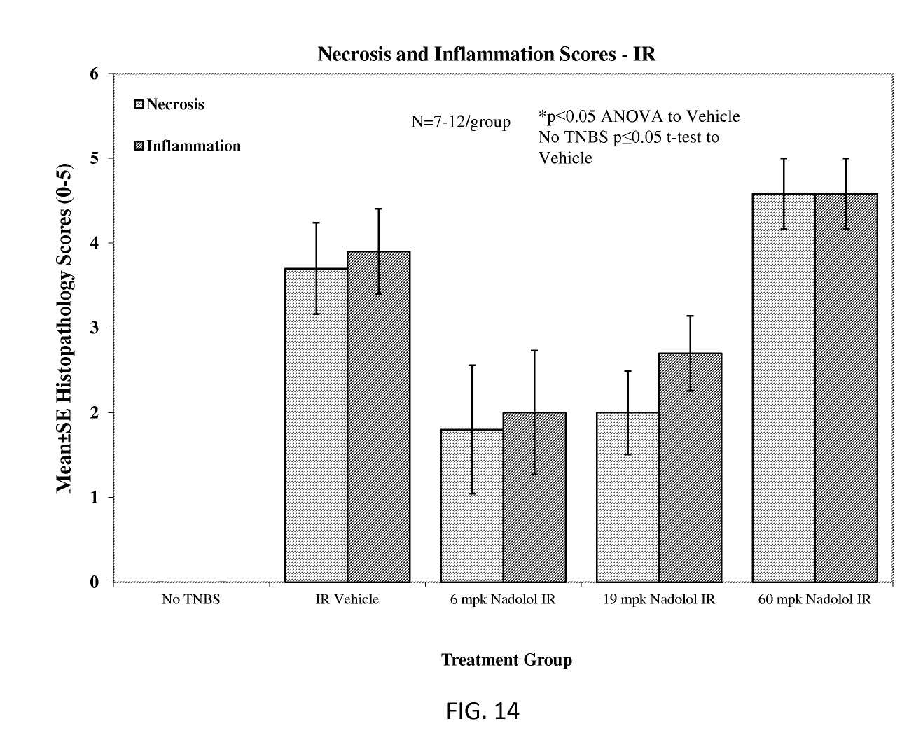

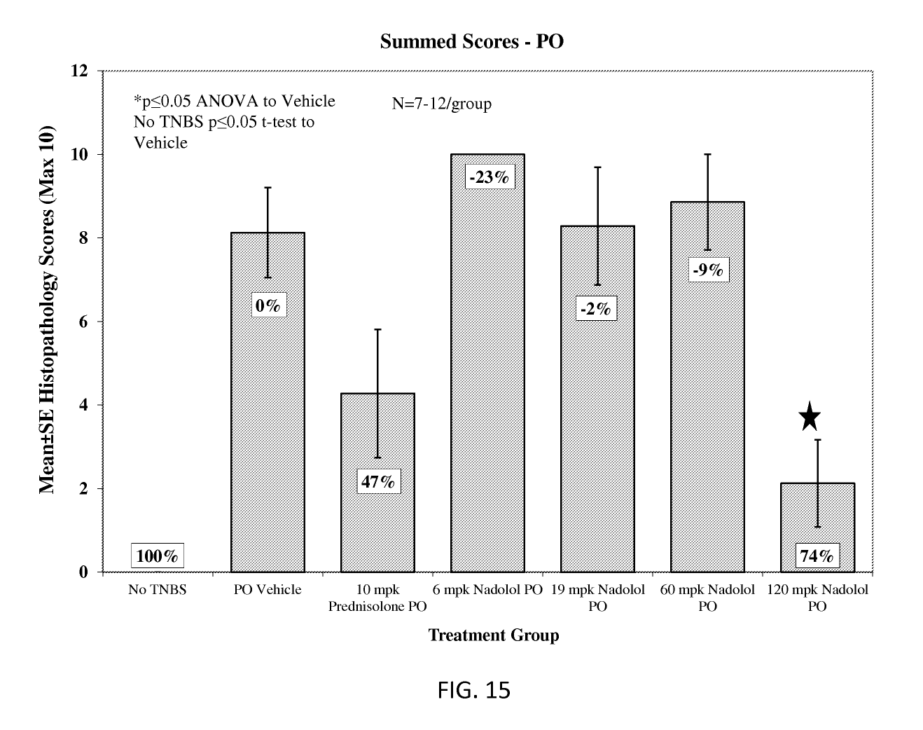

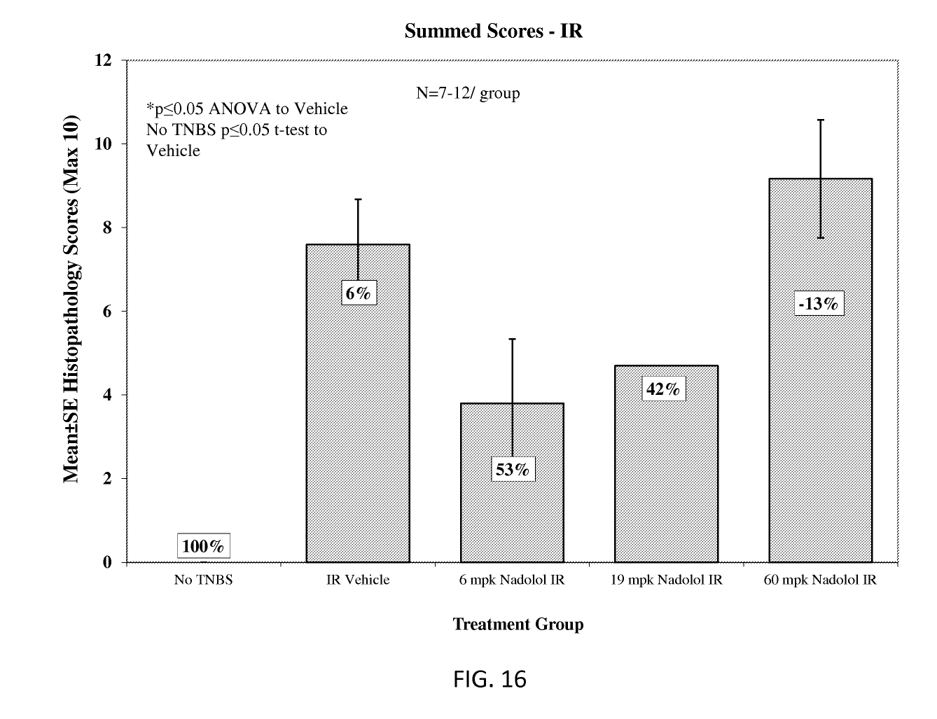

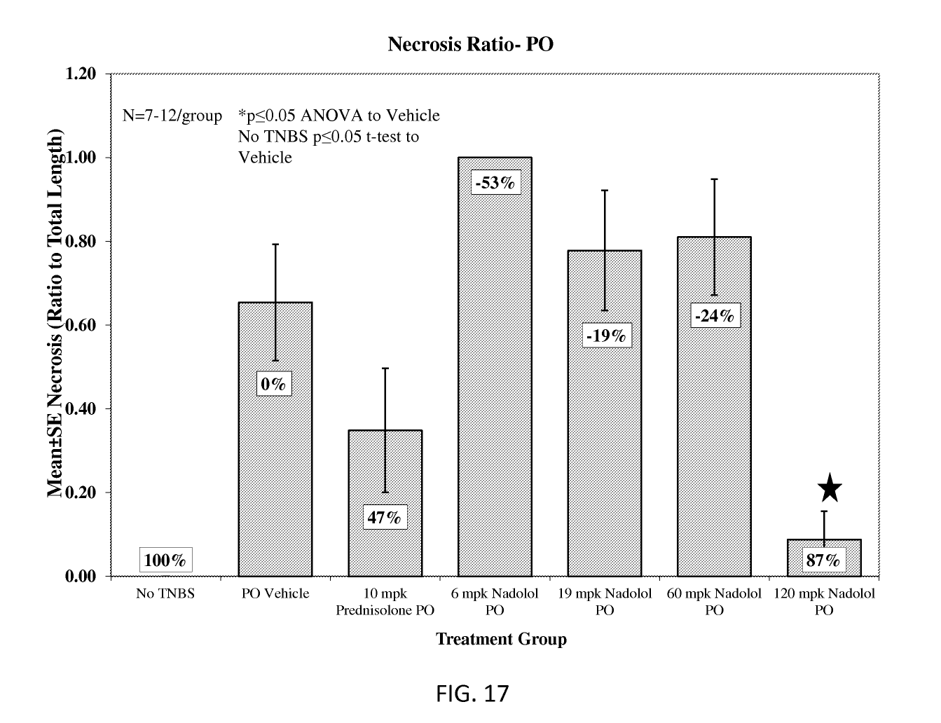

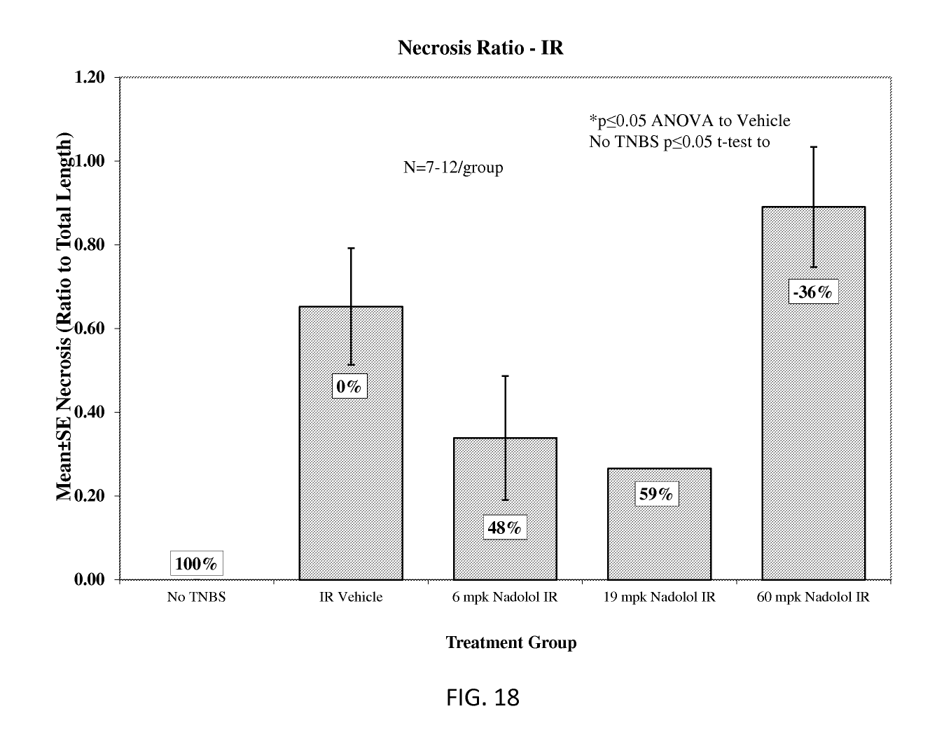

A section of colon is preserved in 10 volumes of 10% neutral buffered formalin for histopathology. A section of colon is homogenized in PBS, centrifuged, and aliquots of the homogenate in labeled Eppendorf tubes are stored at −80° C. for cytokine analysis. The results of this study were recorded in displayed in FIGS. 9-18.

### Example 5

### Pharmakokinetics of Nadolol was Determined in Diseased and Healthy Rats

48 Sprague-Dawley rats (male, 180-200 g) were assigned to 8 groups of 6 rats per group so that each group has approximately the same mean weight.

(DAY −1) Rats were deprived of food 24 hours prior to disease initiation.

(DAY 0) TNBS Solution (50% TNBS/50% 200 proof ethanol; 16 mg/ml TNBS) was prepared. Rats in Groups 3, 4, 7 and 8 were administered TNBS: rats were anesthetized and administered 64 mg/kg (4 ml/kg) TNBS Solution into colon of each rat then rats were refed.

(DAY 6) Rats were bled for pre-dose samples by retro-orbital bleeds. Rats were weighed and dosed as in TABLE 4. Intra-rectal (IR) administration was accomplished by briefly anesthetizing rats, administering the test material at 10 ml/kg via a feeding needle inserted via the rectum, maintaining the rat in an inverted position with anus pinched shut until recovered from anesthesia. Oral formulations (PO) were first dissolved in 0.05N acetic acid and pH adjusted to 6-7 with 0.1N NaOH.

TABLE 4
Treatment Regimen

| Group | # rats | Treatment | Dose (mg/kg) | ROA |
|---|---|---|---|---|
| 1 | 6 | NOTNBS Nadolol | 19 | PO |
| 2 | 6 | NO TNBS Nadolol | 120 | PO |
| 3 | 6 | TNBS Nadolol | 19 | PO |
| 4 | 6 | TNBS Nadolol | 120 | PO |
| 5 | 6 | NO TNBS Nadolol | 19 | IR |
| 6 | 6 | NO TNBS Nadolol | 120 | IR |
| 7 | 6 | TNBS Nadolol | 19 | IR |
| 8 | 6 | TNBS Nadolol | 120 | IR |

*PO = oral gavage
**IR = intra-rectal

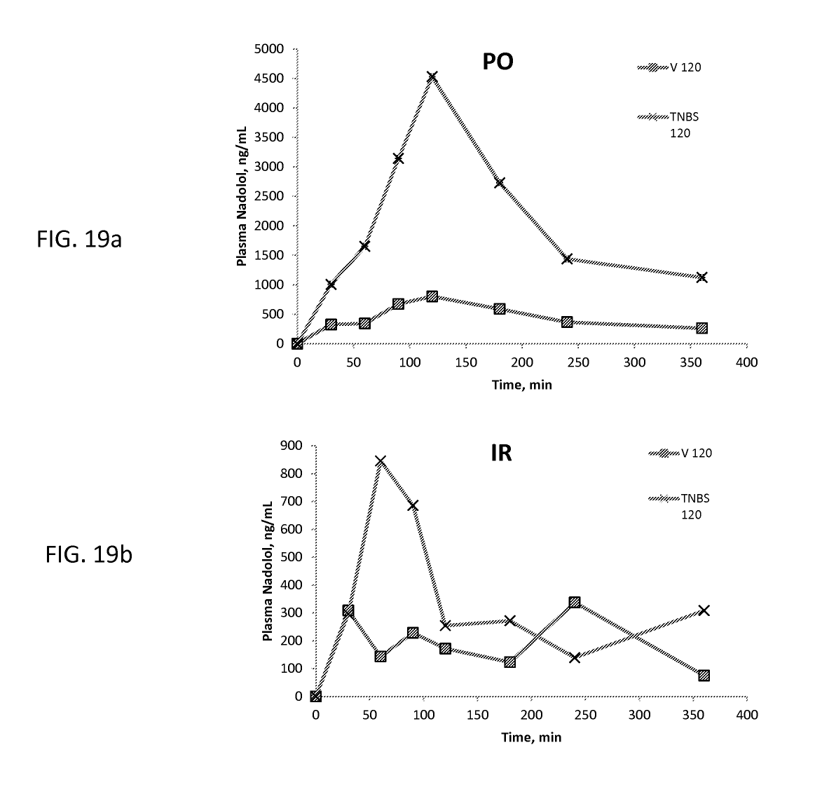

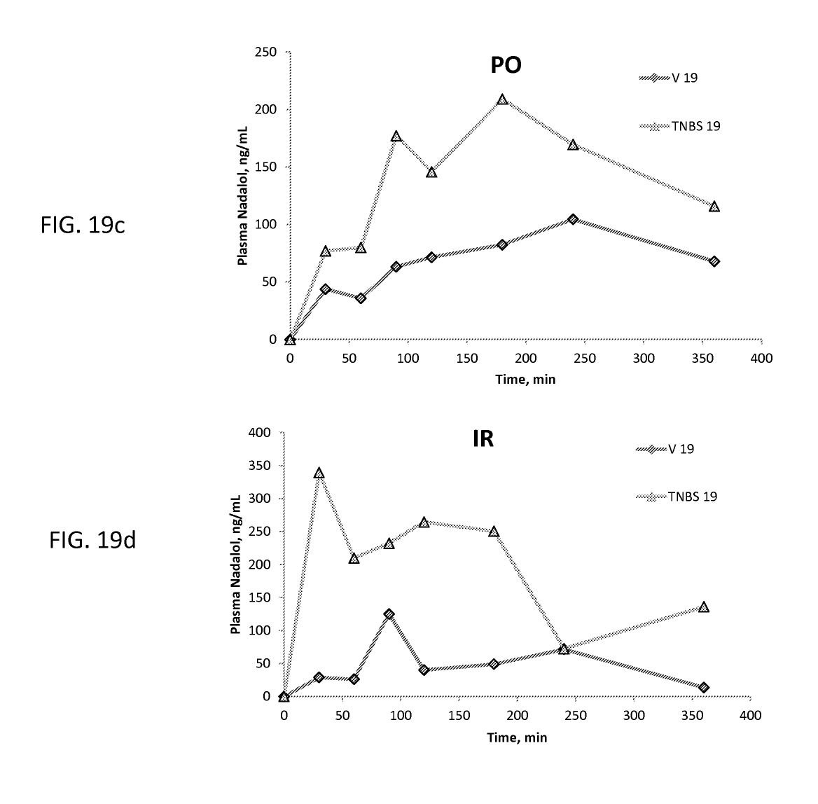

Blood samples were collected from rats by retro-orbital bleed at time points: 30, 60, 90, 120, 180, 240 and 360 minutes post-dose. Samples were collected in pre-chilled-tubes and processed to EDTA plasma. The plasma was aliquoted to four aliquots and stored immediately at −80° C. Each aliquot (50-100 uL) was warmed to room temperature and the plasma level of Nadolol was measured. The results of this pharmacokinetic study are presented in FIG. 19.

### Example 6

### Oral Beta Blocker Efficacy Study

80 Sprague-Dawley (SD) rats (male, 180-200 g) were assigned to 7 groups of 10-12 rats per group so that each group had approximately the same mean weight.

(Day −1): Groups 1-7 were dosed as described in TABLE 5.

TABLE 5
Group Treatments

| Group | # rats | Treatment | Dose (mg/kg) | ROA |
|---|---|---|---|---|
| 1 | 10 | Vehicle/NO TNBS | 10 ml/kg | PO |
| 2 | 12 | Vehicle | 10 ml/kg | PO |
| 3 | 12 | Prednisolone | 10 | PO |
| 4 | 12 | Nadolol | 120 | PO |
| 5 | 12 | Timolol maleate | 120 | PO |
| 6 | 12 | Propranolol HCl | 120 | PO |
| 7 | 12 | Betaxolol | 120 | PO |

PO = oral gavage

Rats were deprived of food for 24 hours prior to disease initiation on Day 0.
Day 0: TNBS solution (50% TNBS: 50% 200 proof ethanol; 16 mg/ml TNBS). Rats in groups 2-8 were administered TNBS solution (Group 1 is NOT treated with TNBS): rats anesthetized, feeding needle inserted into rat anus, 64 mg/kg (4 mL/kg) TNBS solution added to colon of each rat, feeding needle removed, anus closed, rat maintained head down position until recovery from anesthesia, then rats re-fed.
Animals were dosed once daily DAYS −1-7 according to the group treatments listed in Table 5.

On DAY 7, Rats were dosed as in TABLE 5, then blood samples were collected from the anesthetized rats. A midline incision was made on each animal, the colon was removed and length measured and recorded, a longitudinal incision was made on each colon, fecal material was gently washed away with saline, excess fluid was gently blotted and the colon weighed. The severity of IBD was assessed using the following scoring system (maximum score=10):
1) Adhesions:
   none=0
   minimal=1
   involving several bowel loops=2
2) Strictures:
   none=0
   mild=1
   moderate=2
   severe, proximal dilatation=3
3) Ulcers:
   none=0
   inear ulceration <1 cm=1
   two linear ulcers <1 cm=2
   more sites of ulceration or one large ulcer=3
4) Wall thickness:
   less than 1 mm=0
   1-3 mm=1
   >3 mm=2

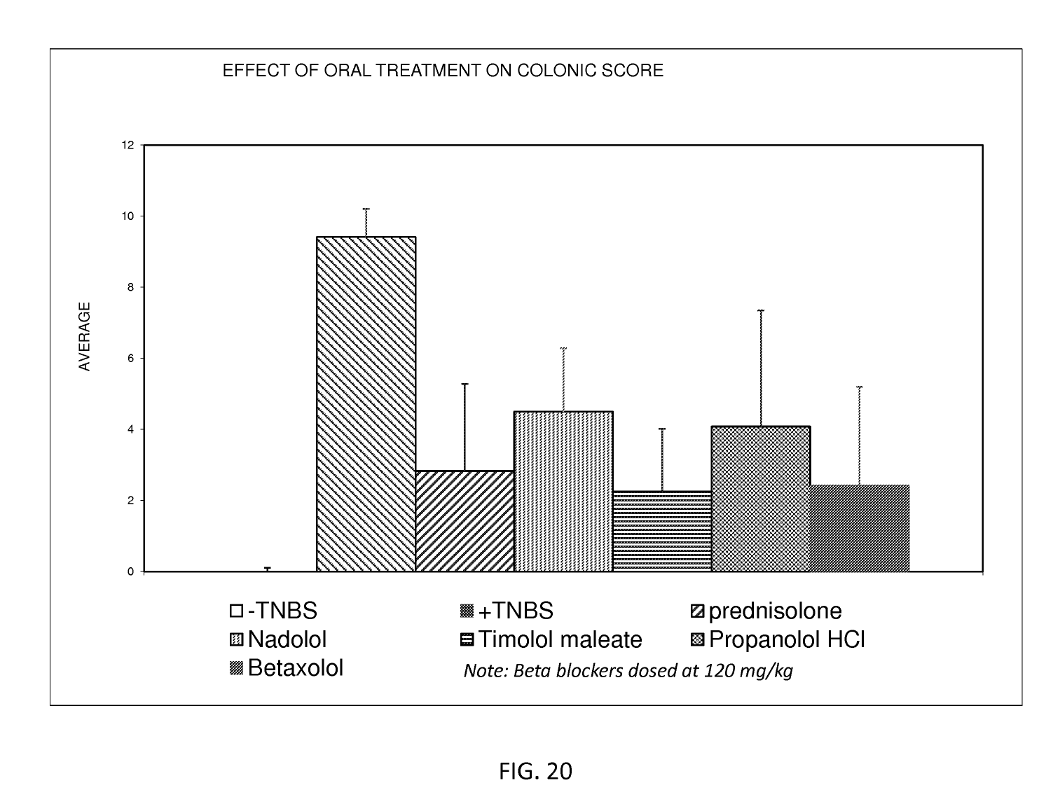

A section of colon was preserved in 10 volumes of 10% neutral buffered formalin for histopathology. A section of the colon is homogenized in PBS, centrifuged, and aliquots of the homogentat were frozen and stored for cytokine analysis. The results of this study were recorded and displayed in FIG. 20.

### Example 7

### A Rectal Beta Blocker Efficacy Study was Conducted

80 Sprague-Dawley (SD) rats (male, 180-200 g) were assigned to 7 groups of 10-12 rats per group so that each group had approximately the same mean weight.

(Day −1): Groups 1-7 were dosed as described in TABLE 6.

TABLE 6
Group Treatments

| Group | # rats | Treatment | Dose (mg/kg) | ROA |
|---|---|---|---|---|
| 1 | 10 | Vehicle/NO TNBS | 10 ml/kg | IR |
| 2 | 12 | Vehicle | 10 ml/kg | IR |
| 3 | 12 | Prednisolone | 10 | PO |
| 4 | 12 | Nadolol | 6 | IR |
| 5 | 12 | Timolol maleate | 6 | IR |
| 6 | 12 | Propranolol HCl | 6 | IR |
| 7 | 12 | Betaxolol | 6 | IR |

PO = oral gavage
IR = intra-rectal administration under anesthesia

Rats were deprived of food for 24 hours prior to disease initiation on Day 0.

Day 0: TNBS solution (50% TNBS: 50% 200 proof ethanol; 16 mg/ml TNBS). Rats in groups 2-8 were administered TNBS solution (Group 1 is NOT treated with TNBS): rats anesthetized, feeding needle inserted into rat anus, 64 mg/kg (4 mL/kg) TNBS solution added to colon of each rat, feeding needle removed, anus closed, rat maintained head down position until recovery from anesthesia, then rats re-fed.

Animals were dosed once daily DAYS −1-7 according to the group treatments listed in Table 6.

On DAY 7, Rats were dosed as in TABLE 6, then blood samples were collected from the anesthetized rats. A midline incision was made on each animal, the colon was removed and length measured and recorded, a longitudinal incision was made on each colon, fecal material was gently washed away with saline, excess fluid was gently blotted and the colon weighed. The severity of IBD was assessed using the following scoring system: maximum score=10
1) Adhesions:
   none=0
   minimal=1
   involving several bowel loops=2
2) Strictures:
   none=0
   mild=1
   moderate=2
   severe, proximal dilatation=3
3) Ulcers:
   none=0
   inear ulceration <1 cm=1
   two linear ulcers <1 cm=2
   more sites of ulceration or one large ulcer=3
4) Wall thickness:
   less than 1 mm=0
   1-3 mm=1
   >3 mm=2

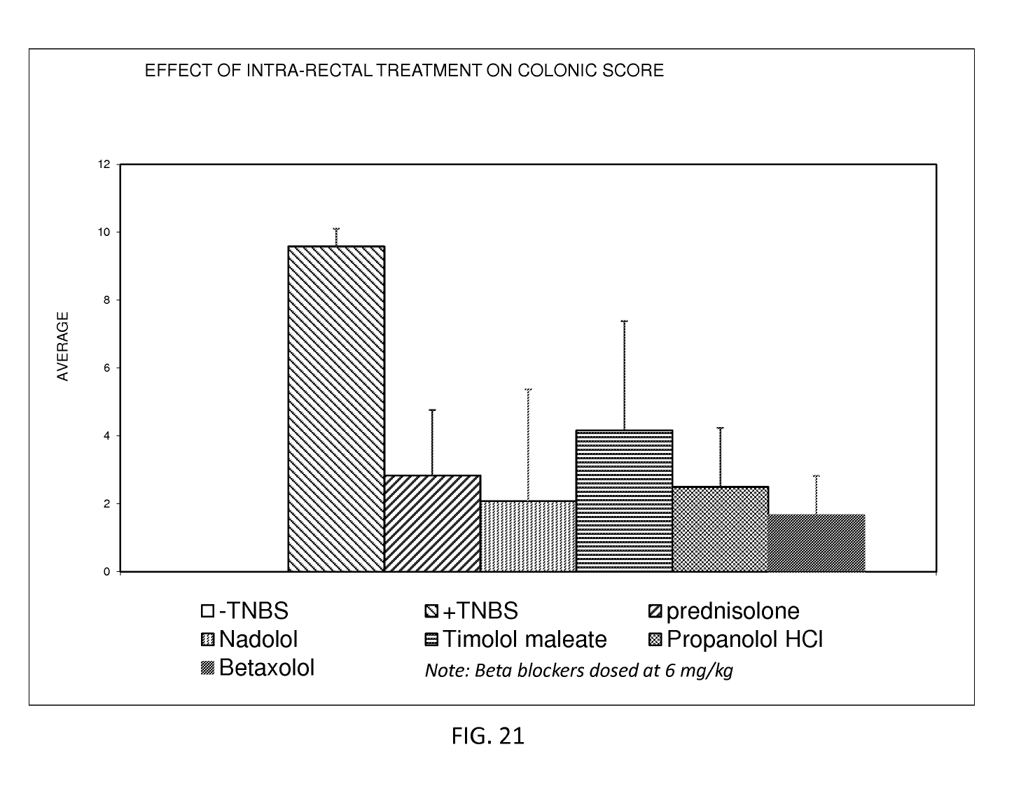

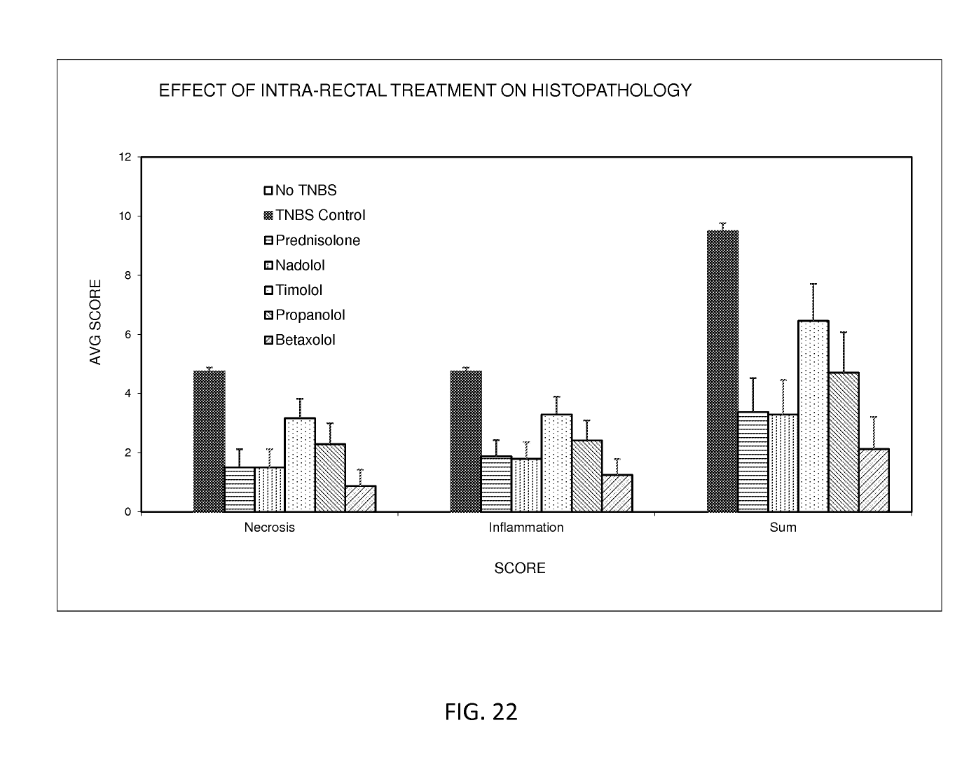

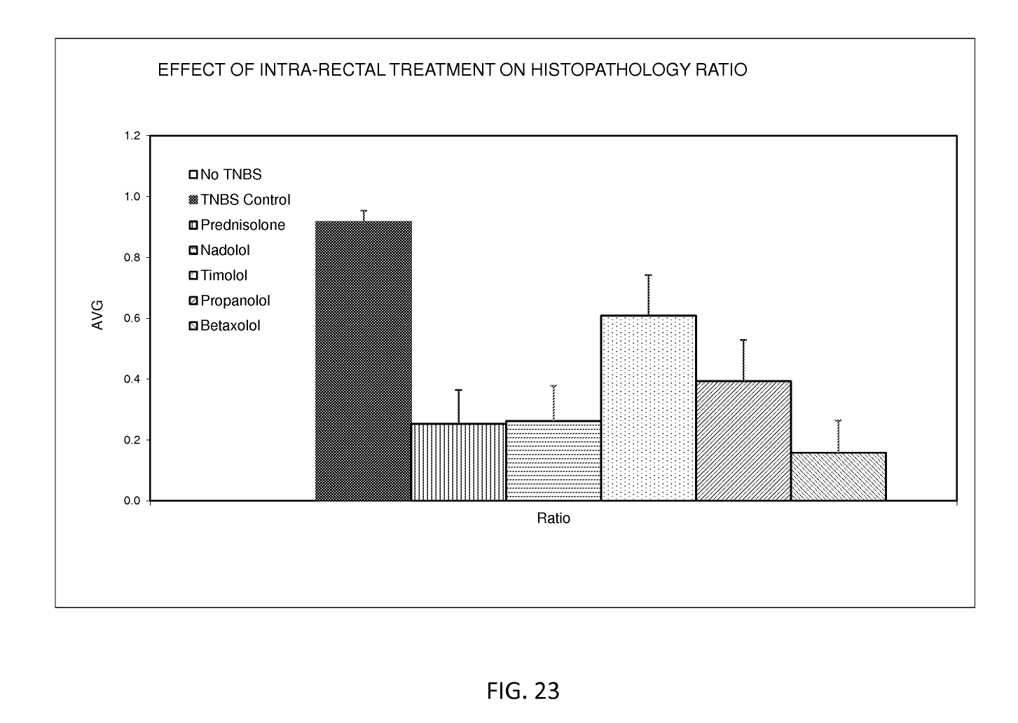

A section of colon was preserved in 10 volumes of 10% neutral buffered formalin for histopathology. A section of the colon is homogenized in PBS, centrifuged, and aliquots of the homogentat were frozen and stored for cytokine analysis. The results of this study were recorded and displayed in FIG. 21-23.

The present invention may be embodied in other specific forms without departing from the spirit or essential attributes thereof.

1. A method of treating a gastrointestinal disease in a subject in need thereof, the method comprising administering to the subject a therapeutically-effective amount of a beta blocker.

2-83. (canceled)

84. The method of claim 1, wherein the beta blocker is nadolol.

85. The method of claim 1, wherein the beta blocker is timolol.

86. The method of claim 1, wherein the beta blocker is atenolol.

87. The method of claim 1, wherein the beta blocker is acebutolol.

88. The method of claim 1, wherein the beta blocker is metoprolol.

89. The method of claim 1, wherein the beta blocker is labetalol.

90. The method of claim 1, wherein the beta blocker is oxprenolol.

91. The method of claim 1, wherein the beta blocker is pindolol.

92. The method of claim 1, wherein the beta blocker is propanolol.

93. The method of claim 1, wherein the beta blocker is sotalol.

94. The method of claim 1, wherein the beta blocker is bisoprolol.

95. The method of claim 1, wherein the beta blocker is alprenolol.

96. The method of claim 1, wherein the beta blocker is propranolol.

97. The method of claim 1, wherein the beta blocker is betaxolol.

98. The method of claim 1, wherein the gastrointestinal disease is an inflammatory disease.

99. The method of claim 1, wherein the gastrointestinal disease is an inflammatory bowel disease.

100. The method of claim 1, wherein the gastrointestinal disease is Crohn's disease.

101. The method of claim 1, wherein the gastrointestinal disease is colitis.

102. The method of claim 1, wherein the gastrointestinal disease is ulcerative colitis.

103. The method of claim 1, wherein the gastrointestinal disease is constipation.

104. The method of claim 1, wherein the gastrointestinal disease is diarrhea.

105. The method of claim 1, wherein the gastrointestinal disease is a motility disorder.

106. The method of claim 1, wherein the gastrointestinal disease is an Irritable Bowel Syndrome (IBS).

107. The method of claim 1, wherein the gastrointestinal disease is a disorder of a muscle of the digestive tract.

108. The method of claim 1, wherein the gastrointestinal disease is a disorder of a nerve of the digestive tract.

109. The method of claim 1, wherein the beta blocker is released in the small intestine of the subject.

110. The method of claim 1, wherein the beta blocker is released in the large intestine of the subject.

111. The method of claim 1, wherein the administration is application to the intestine of the subject.

112. The method of claim 1, wherein the administration is topical.

113. The method of claim 1, the administration is oral.

114. The method of claim 1, wherein the administration is intra-rectal.

115. The method of claim 1, wherein the therapeutically-effective amount is from 1 mg to 40 mg.

116. The method of claim 1, wherein the therapeutically-effective amount is from 1 mg to 600 mg.

117. The method of claim 1, wherein the beta blocker blocks a beta-1 adrenergic receptor in the subject.

118. The method of claim 1, wherein the beta blocker blocks a beta-2 adrenergic receptor in the subject.

119. The method of claim 1, wherein the beta blocker blocks a beta-1 adrenergic receptor and a beta-2 adrenergic receptor in the subject with about the same potency.

120. The method of claim 1, wherein the subject is a human.

121. The method of claim 120, wherein the gastrointestinal disease is an inflammatory disease, the administration is topical, the therapeutically-effective amount is from 1 mg to 600 mg, and the beta blocker is released in the large intestine of the subject.

122. The method of claim 121, wherein the beta blocker blocks a beta-1 adrenergic receptor and a beta-2 adrenergic receptor in the subject with about the same efficiency.

123. The method of claim 122, wherein the gastrointestinal disease is colitis.

124. The method of claim 122, wherein the gastrointestinal disease is ulcerative colitis.

125. A method of treating an inflammatory gastrointestinal disease in a human in need thereof, the method comprising topically administering to the subject a beta blocker in an amount of 1 mg to 600 mg, wherein the beta blocker blocks a beta-1 adrenergic receptor and a beta-2 adrenergic receptor in the subject with about the same efficiency, wherein the beta blocker is released in the large intestine of the human.

126. The method of claim 125, wherein the inflammatory gastrointestinal disease is colitis.

127. The method of claim 125, wherein the gastrointestinal disease is ulcerative colitis.
